(19) United States
(12) Patent Application Publication
(10) Pub. No.: US 2024/0252726 A1
(43) Pub. Date: Aug. 1, 2024

(54) Engineered Neural Networks in Tailored Hydrogel Sheaths and Methods for Manufacturing the Same

(71) Applicants: THE TRUSTEES OF THE UNIVERSITY OF PENNSYLVANIA, Philadelphia, PA (US); The United States of America as Represented by the Department of Veterans Affairs, Washington, DC (US)

(72) Inventors: Daniel Kacy Cullen, Media, PA (US); Wisberty J. Gordian-Velez, Philadelphia, PA (US); H. Isaac Chen, Penn Valley, PA (US); Jason A. Burdick, Philadelphia, PA (US)

(21) Appl. No.: 18/561,507

(22) PCT Filed: May 18, 2022
(86) PCT No.: PCT/US22/29908

§ 371 Date: Nov. 16, 2023

Related U.S. Application Data
(60) Provisional application No. 63/190,581, filed on May 19, 2021.

(51) Int. Cl. A61L 27/52; A61K 35/30; A61L 27/20; A61L 27/36; A61L 27/38; A61L 27/58; G06N 3/06

(52) U.S. Cl. CPC A61L 27/52; A61K 35/30; A61L 27/20; A61L 27/3633; A61L 27/3675; A61L 27/383; A61L 27/3878; A61L 27/58; G06N 3/061

(57) ABSTRACT

In various aspects and embodiments the present disclosure provides a construct comprising a pre-formed neural network, the construct comprising a micro-column comprising an outer sheath comprising a hyaluronic acid (HA) hydrogel, and a core comprising an extracellular matrix (ECM); a plurality of neurons within the micro-column. The present disclosure further provides methods of making and using the same.

### CROSS-REFERENCE TO RELATED APPLICATION

The present application is entitled to priority under 35 U.S.C. § 119(e) to U.S. Provisional Patent Application No. 63/190,581 filed May 19, 2021, the content of which is incorporated by reference herein in its entirety.

### STATEMENT REGARDING FEDERALLY SPONSORED RESEARCH OR DEVELOPMENT

This invention was made with government support under I01-BX003748 awarded by Department of Veterans Affairs, 1845298 awarded by the National Science Foundation, IK2-RX002013 awarded by the Department of Veterans Affairs, U01 NS094340 awarded by the National Institutes of Health. The government has certain rights in the invention.

### BACKGROUND

Parkinson's disease (PD) is a disorder characterized by motor deficits, such as tremors, slowness of movement (bradykinesia), and rigidity, as well as non-motor symptoms including autonomic dysfunction, depression, and dementia. PD is the second most common neurodegenerative disease, affecting the quality of life of 10 million people worldwide, including 60,000 new cases per year in the United States and 1-2% of people 65 years or older. Projections indicate that the number of PD patients in the ten most populated countries will double by 2030 to 9.3 million. The Parkinson's Foundation has estimated that the total cost associated with treatment in the United States is $52 billion/year, including $2,500/year for medications and $100,000 for surgery per patient. Existing therapies can mitigate symptoms for a limited time, but there are currently no approved treatments to prevent or repair the underlying pathology. The search for an effective treatment is thus a crucial research endeavor. A key aspect of PD pathology is the loss of dopaminergic neurons in the substantia nigra pars compacta (SNpc) and the degeneration of their axonal projections along the nigrostriatal pathway. These dopaminergic axons synapse with medium spiny neurons (MSNs) in the dorsolateral striatum, a brain region associated with motor movements, decision making, and learning. In healthy patients, activated dopaminergic neurons release dopamine, which acts on MSNs expressing dopamine receptors. Through the direct and indirect pathways involving dopamine receptor 1 and 2, respectively, these synaptic connections modulate the gamma aminobutyric acid (GABA)-dependent inhibitory output of the basal ganglia (i.e., globus pallidus internus and the substantia nigra pars reticulata (SNpr)). With the loss of dopamine released by nigrostriatal terminals in PD, the net effect is less suppression of the basal ganglia by the striatum, increased inhibition of the thalamus, and reduced activation of the motor cortex. A loss of ˜30-50% of dopaminergic neurons and a reduction of 50-60% in striatal dopamine has been associated with the onset of symptoms. Moreover, axon degeneration on its own, years before the death of neuron bodies, has been interpreted as an early sign of PD.

Unfortunately, most patients are diagnosed when motor symptoms are evident and significant disease progression has already occurred. Current treatments focus on managing symptoms with drug therapy or deep brain stimulation (DBS). On the first front, the gold standard has been administration of L-3,4-dihydroxyphenylalanine (L-DOPA), the immediate precursor to dopamine, which has a short half-life and variable absorption and blood-brain barrier transport that prevent having sustained drug delivery. Possibly because of this discontinuous stimulation, long-term patients tend to suffer abnormal muscle movement (dyskinesia) and fluctuations between symptom control and recurrence. As alternatives, dopamine agonists (e.g., apomorphine, rotigotine) mimic the normal interaction of dopamine with receptors in the striatum and tend to have a longer half-life and less pulsatile effects than L-DOPA, but with less potency. On the other hand, DBS typically involves the implantation of electrodes to stimulate the subthalamic nucleus, which can improve motor scores and reduce dependence on drugs. Regardless, electrical stimulation is inherently non-specific, the mechanisms of DBS are unknown, and the effectiveness of electrodes can be affected by chronic foreign body responses. DBS patients may also be subject to bleeding, infection, and psychiatric symptoms. Current treatments also fail to intervene in the death of dopaminergic neurons and the loss of striatal innervation at the core of PD. There is a need in the art for new treatments for Parkinson's disease and other conditions of the nervous system that better satisfy patient needs. The present disclosure addresses this need.

### SUMMARY

Among other things, the present disclosure provides a construct comprising a pre-formed neural network, the construct comprising: a micro-column comprising an outer sheath comprising a hyaluronic acid (HA) hydrogel, and a core comprising an extracellular matrix (ECM); and a plurality of neurons within the micro-column.

In various embodiments, the construct is biocompatible.

In various embodiments, the construct is an implantable construct.

In various embodiments, the hydrogel sheath is cylindrical.

In various embodiments, the ECM core substantially fills a lumen of the hydrogel sheath.

In various embodiments, the micro-column is directed along a substantially straight line along its length.

In various embodiments, the micro-column is directed along a curved path along its length.

In various embodiments, the plurality of neurons have cell bodies substantially localized in proximity to a first end of the micro-column and extend axons longitudinally along at least a portion of a length of the micro-column.

In various embodiments, the plurality of neurons comprise one or more three dimensional aggregates.

In various embodiments, the axons are located within and extend longitudinally along a lumen of the hydrogel sheath from the neurons at the first end and towards the opposite end.

In various embodiments, the axons grow through the ECM of the core and/or along an interface between an inner surface of the hydrogel sheath and the ECM of the core.

In various embodiments, the neurons and axons extending therefrom have a cyto-architecture that replicates long-range axon tracts present in a subject.

In various embodiments, the subject is a human subject.

In various embodiments, the neuronal cells and axons extending therefrom have a cyto-architecture that replicates long-range axon tracts present in a brain of a human subject.

In various embodiments, the neuronal cells and axons extending therefrom have a cyto-architecture that mimics a native axon pathway between the substantia nigra and the striatum in a brain of a subject.

In various embodiments, the axon tracts are or have been pre-directed via the micro-column.

In various embodiments, the neurons together with the axons extending therefrom form a biofabricated micro-tissue.

In various embodiments, the axons of the plurality of neurons extend along at least 50% of the length of the micro-column.

In various embodiments, the axons extend along at least 75% of the length of the micro-column.

In various embodiments, the axons extend along 90% of the length of the micro-column.

In various embodiments, an outer diameter of the micro-column ranges from about 500 microns to about 2,500 microns.

In various embodiments, the outer diameter of the micro-column ranges from about 500 to about 1,500 microns.

In various embodiments, the outer diameter of the micro-column ranges from about 750 to about 1,000 microns.

In various embodiments, the outer diameter is a cross-sectional diameter of the hydrogel sheath.

In various embodiments, the outer diameter is a cross-sectional diameter of the hydrogel sheath and including any outer coatings thereon.

In various embodiments, an inner diameter of the micro-column ranges from about 250 microns to about 2,000 microns.

In various embodiments, the inner diameter of the micro-column ranges from about 250 to about 1,000 microns.

In various embodiments, the inner diameter of the micro-column is about 500 microns.

In various embodiments, the ECM comprises a polysaccharide.

In various embodiments, the ECM comprises one or more members selected from the group consisting of collagen, fibrin, fibronectin, gelatin, hyaluronic acid, laminin, and Matrigel.

In various embodiments, the ECM comprises collagen.

In various embodiments, the ECM comprises collagen at a concentration ranging from about 0.1 to 10 mg/ml.

In various embodiments, the ECM comprises collagen at a concentration of about 1 mg/ml.

In various embodiments, the ECM comprises laminin.

In various embodiments, the ECM comprises laminin at a concentration ranging from about 0.1 to 10 mg/ml.

In various embodiments, the ECM comprises laminin at a concentration of about 1 mg/ml.

In various embodiments, the hyaluronic acid (HA) hydrogel is or comprises a cross-linked modified hyaluronic acid.

In various embodiments, the modified hyaluronic acid is methacrylated HA (MeHA).

In various embodiments, the hyaluronic acid comprises about 0.5 to about 20% wt MeHA.

In various embodiments, the hyaluronic acid is or comprises 3-5% MeHA.

In various embodiments, the modified HA comprises one or more members selected from the group consisting of norbornene-modified HA, acrylated HA, maleimide HA, and hydroxyethyl methacrylate HA.

In various embodiments, the outer hydrogel sheath is a 3D printed and photopolymerized MeHA cylinder.

In various embodiments, the outer hydrogel sheath comprises one or more hydrolysis sensitive compounds within crosslinks.

In various embodiments, the one or more hydrolysis sensitive compounds comprise esters.

In various embodiments, the one or more hydrolysis sensitive compounds comprise lactic acid, caprolactone or an anhydride.

In various embodiments, the outer hydrogel sheath comprises methacrylated hyaluronic acid doped with the one or more hydrolysis sensitive compounds.

In various embodiments, one or more hydrolysis sensitive compounds are located between HA and the methacrylate groups.

In various embodiments, the outer hydrogel sheath comprises one or more di-thiol peptides.

In various embodiments, at least a portion of the one or more di-thiol peptides are sensitive to cleavage.

In various embodiments, at least a portion of the one or more di-thiol peptides are sensitive to cleavage by matrix metalloproteinases expressed by cells.

In various embodiments, the plurality of neurons comprise dopaminergic neurons.

In various embodiments, at least 50% of the plurality of neurons are dopaminergic neurons.

In various embodiments, the dopaminergic neurons are obtained via purification.

In various embodiments, the dopaminergic neurons comprise midbrain dopaminergic neurons.

In various embodiments, the midbrain dopaminergic neurons comprise A9 neurons.

In various embodiments, the plurality of neurons comprise GABAergic neurons.

In various embodiments, at least 50% of the plurality of neurons are GABAergic neurons.

In various embodiments, the GABAergic neurons are obtained via purification.

In various embodiments, the plurality of neurons comprise glutaminergic neurons.

In various embodiments, at least 50% of the plurality of neurons are glutaminergic neurons.

In various embodiments, the glutaminergic neurons are obtained via purification.

In various embodiments, the plurality of neurons comprise cholinergic neurons.

In various embodiments, at least 50% of the plurality of neurons are cholinergic neurons.

In various embodiments, the cholinergic neurons are obtained via purification.

In various embodiments, the plurality of neurons comprise midbrain neurons.

In various embodiments, the plurality of neurons comprise human neurons.

In various embodiments, the human neurons comprise induced pluripotent stem cell (iPSC)-derived neurons.

In various embodiments, the plurality of neurons comprise human A9 dopaminergic neurons.

In various embodiments, the human A9 dopaminergic neurons comprise stem cell-derived human A9 dopaminergic neurons.

In various embodiments, the stem cells from which the A9 dopaminergic neurons are derived are induced pluripotent stem cells (iPSCs).

In various embodiments, the plurality of neurons comprise at least 50,000 neurons.

In various embodiments, the plurality of neurons comprise at least 100,000 neurons.

In various embodiments, the plurality of neurons comprise at least 125,000 neurons.

In various embodiments, the plurality of neurons comprise at least 500,000 neurons.

In various embodiments, the plurality of neurons comprise at least 1,000,000 neurons.

In various embodiments, the micro-column has a length ranging from about 2 to about 5 centimeters.

In various embodiments, axons of the plurality of neurons extend a distance ranging from about 2 to about 5 centimeters along the length of the micro-column.

In various embodiments, the micro-column is adapted for implantation along a trajectory encompassing a substantia nigra (SN) region and a striatum region of a subject.

In various embodiments, the SN region is a ventrolateral SN region.

In various embodiments, the striatum region is a dorsal striatum region.

In various embodiments, the subject is a human subject.

In various embodiments, neurons comprise A9 dopaminergic neurons and exhibit dopamine release of at least 50 nM as measured by fast scan cyclic voltammetry.

In various embodiments, the plurality of neurons releases and/or has a quantity of dopaminergic neurons sufficient to release dopamine at a level sufficient to provide for a level of at least 4 ng/mg in tissue.

In various embodiments, the dopaminergic neurons release dopamine at a level sufficient to provide for a level of at least 4 ng/mg in tissue within six weeks after implantation in a subject.

In various embodiments, the plurality of neurons provides for increase in 18F-DOPA uptake in a putamen of a subject at a level of about 50-60% of a normal value.

In various embodiments, the neurons provide for an increase in 18F-DOPA uptake in the putamen of the subject at the level of about 50-60% of a normal value upon implantation in the subject.

In various embodiments, an increase at the level of about 50-60% of a normal value is achieved.

In another aspect, the present disclosure provides a method of manufacturing a construct comprising a pre-formed neural network, the method comprising:
(a) seeding a first end of a micro-column with a plurality of neural precursor cells and/or dopaminergic neurons; and
(b) culturing the micro-column and plurality of neural cells seeded therein in-vitro.

In various embodiments, the construct is a biocompatible construct.

In various embodiments, the construct is an implantable construct.

In various embodiments, the construct is for use in an in-vitro test bed.

In various embodiments, step (b) comprises causing growth of axons from the neural cells, along a length of the micro-column, toward a second, opposite, end of the micro-column.

In various embodiments, the method comprises:
(c) determining axons growth from the plurality of neural cells has reached a particular length; and
(d) responsive to the particular length of axon growth being determined to have been reached, packaging and/or providing the micro-column for implantation.

In various embodiments, the particular length is a predetermined desired length.

In various embodiments, the particular length ranges from about 2 to about 5 centimeters.

In various embodiments, step (c) comprises imaging the micro-columns and neural cells therein.

In various embodiments, step (c) comprises imaging via microscopy, fast-scan cyclic voltammetry (FSCV), staining, sectioning or measuring axon density.

In various embodiments, the plurality of neural cells with which the micro-column is seeded at step (a) comprise neural cell aggregates.

In various embodiments, the neural cell aggregates comprise a plurality of approximately spherical aggregates of neural cells.

In various embodiments, each neural cell aggregate comprises cells at a density ranging from about 100,000 to about 300,000 neurons per aggregate.

In various embodiments, a plurality of the neural cell aggregates exhibit a diameter of at least 500 μm.

In various embodiments, the micro-column comprises a hydrogel sheath and a core comprising an extracellular matrix (ECM), and wherein the neural cells are seeded to be in direct contact with the ECM of the core.

In various embodiments, the hydrogel sheath comprises MeHA.

In various embodiments, the hydrogel sheath of the micro-column is a 3D printed cylinder.

In various embodiments, the method comprises 3D printing the hydrogel sheath prior to step (a).

In various embodiments, the method comprises differentiating human induced pluripotent stem cells (iPSCs) for a particular differentiation period prior to step (a), thereby producing differentiated cells and, following differentiating the iPSCs for the particular differentiation period, performing step (a) using the differentiated cells as the neural cells

In various embodiments, the method comprises seeding the differentiated iPSCs in the micro-column after about 40 dd.

In various embodiments, the method comprises seeding the differentiated iPSCs in the micro-column after about 11 to about 20 dd.

In various embodiments, the method comprises seeding the differentiated iPSCs in the micro-column once dopaminergic precursor fate is established and when the cells are usually replanted and matured further.

In various embodiments, the micro-column and or cells have one or more features articulated in the preceding embodiments.

In another aspect, the present disclosure provides an in-vitro test bed comprising: the construct as described herein, and further comprising a first population of neurons and axons grown therefrom; and a second population of neurons, synapsed with the first population.

In various embodiments, the second population of neurons comprise striatal neurons.

In various embodiments, the second population of neurons are seeded at an end of the construct opposite to the end at which the first population of neurons were seeded.

In various embodiments, axons from the first population of neurons extend longitudinally from a first end of the construct along a length of the construct and synapse with the second population seeded at a second, opposite, end of the construct.

In various embodiments, cell bodies of the first population are localized in substantial proximity to the first end of the construct.

In another aspect, the present disclosure provides a method of at least partially replacing a population of neurons forming a pathway between the substantia nigra and striatum in a subject, the method comprising implanting at least one construct as described herein into a brain of the subject.

In various embodiments, the method comprises ameliorating one or more conditions of the subject.

In various embodiments, ameliorating the one or more conditions comprises restoring motor function of the subject.

In various embodiments, the ameliorating the one or more conditions comprises reducing pain of the subject.

In various embodiments, the ameliorating the one or more conditions comprises reducing tremors of the subject.

In various embodiments, the method comprises implanting the at least a portion of one construct within a substantia nigra of the subject.

In various embodiments, following implantation, the neurons of the construct synapse with host neurons in a brain of the subject.

In various embodiments, the host neurons with which the neurons of the construct synapse comprise, medium spiny neurons (MSNs) in a dorsolateral striatum of the subject.

In various embodiments, the subject is a human subject.

In various embodiments, implanting the at least one construct comprises using MRI-guided neurosurgery.

In various embodiments, implanting the at least one construct comprises implanting a plurality of constructs.

In various embodiments, the implanting the plurality of constructs comprises implanting a plurality of constructs in a single hemisphere of the brain of the subject.

In various embodiments, implanting the plurality of constructs comprises implanting one or more constructs in each hemisphere of the brain of the subject.

In various embodiments, the method comprises implanting 1 to 3 constructs in each hemisphere of the brain of the subject.

In various embodiments, implantation in a first hemisphere is performed via a first surgery and implantation in a second hemisphere is performed via a second surgery, performed at a different time than the first surgery.

In various embodiments, the second surgery is performed about 6 months after the first surgery.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIGS. 1A-1E: Hyaluronic acid-encased, human tissue-engineered nigrostriatal pathway (TE-NSP) concept and current fabrication method. FIG. 1A: TE-NSPs consisted of an aggregate of dopaminergic neurons, sourced from human iPSCs, extending long axonal projections throughout the extracellular matrix core of a hydrogel micro-column encasement. FIG. 1B: Dopaminergic neurons were obtained from the floor-plate midbrain differentiation of human iPSCs after ˜40 days. A chosen volume of dissociated cell solution was added to custom-made micro-wells, centrifuged, and incubated overnight to form an aggregate of neurons. FIG. 1C: In parallel to neuronal differentiation, the hydrogel encasement for TE-NSPs was made by drawing a MeHA/photoinitiator solution inside a capillary tube containing an acupuncture needle. The MeHA schematic shows the modified HA chains, with the double bonds of the methacrylate groups shown. Then, the MeHA solution inside the tubes was gelled by exposure to UV light, which results in the formation of crosslinks between methacrylate groups. The needle and tube were then removed to obtain the MeHA hydrogel micro-column, with its OD and ID controlled by the choice of tube and needle, respectively. FIG. 1D: The previous steps converged when adding and polymerizing a collagen and laminin solution within the lumen of the micro-columns and then seeding an aggregate inside one end using fine forceps. The constructs were then cultured for the desired time to promote axon growth. FIG. 1E: TE-NSPs may be implanted to span the region between the substantia nigra (SN) and the striatum, thus restoring the nigrostriatal pathway. These constructs may extend axons into the striatum and synapse with regions relevant for PD treatment. Moreover, the neurons in the aggregate may integrate with the circuitry remaining in and modulating the SN to effectively close the loop of the nigrostriatal circuit.

FIGS. 2A-2N: Growth, phenotype, cytoarchitecture, and functionality of MeHA-encased rat TE-NSPs. FIG. 2A: Photorheology profile shows the change in the storage (G′) and loss (G″) moduli of 3% and 5% MeHA solutions after exposure to UV light at 2 min. FIGS. 2B and 2C: The compressive moduli were calculated from the slope of the stress vs compressive strain curves. Data presented as mean±SEM (*P<0.05, *** P<0.001, **** P<0.0001). FIGS. 2D and 2E: The growth lengths and rates for neurites extended by rat embryonic ventral midbrain aggregates were quantified as a function of time in 1% agarose (n=7), 3% MeHA (n=13), or 5% MeHA (n=14) micro-columns (OD: 398 μm, ID: 160 μm, length: ˜0.5 cm, collagen+laminin core). Repeated measures two-way ANOVA yielded a significant effect for the biomaterial over length (P=0.0002) and rate (P=0.0011) and for time over length and rate (P<0.0001). Data presented as mean±SEM (*P<0.05, ** P<0.01 from Tukey's multiple comparisons test; asterisks indicate significant differences for 3% and 5% MeHA relative to 1% agarose, respectively; #represents the comparison between 5% and 3% MeHA with P<0.05). FIG. 2F: Phase-contrast image of a representative 3% MeHA TE-NSP at 14 DIV. FIG. 2G: Maximum intensity projection of confocal z-stacks after staining the construct in FIG. 2F for axons (β-tubulin III), dopaminergic neurons (tyrosine hydroxylase (TH)), and nuclei (Hoechst). FIGS. 2H and 2I: Higher magnification images of the aggregate and axon tract regions, respectively. FIG. 2J: At 37 DIV, rat TE-NSPs were analyzed with FSCV for electrically-evoked dopamine release. A carbon fiber electrode was inserted into the neuronal aggregate spanned by a stimulating electrode. FIG. 2K: The effect of biomaterial encasement on dopamine release was evaluated at the same time point, with no significant differences (P=0.6175). FIG. 2L-2N: Concentration traces (top), cyclic voltammograms (CV) at peak release (center), and CV colorplots (bottom) for constructs in 1% agarose and 3% and 5% MeHA. The black bar represents the time of stimulation. The plots indicate the release of dopamine, as assessed from characteristic oxidation and reduction currents around 0.6 V and −0.3 V, respectively. Scale bars: FIG. 2F: 250 μm; FIG. 2G: 200 μm; FIGS. 2H and 2I: 50 μm.

FIGS. 3A-3D: Implantation of MeHA-encased rat TE-NSPs. FIG. 3A: Confocal image of a GFP+ rat TE-NSP encased in 3% MeHA (OD: 398 μm, ID: 160 μm, length: 0.6 cm) and implanted for 2 weeks in an adult male rat and stained for all neurons (GFP) and dopaminergic neurons (TH). FIGS. 3B and 3C: Magnification of areas of the implant within the striatum and adjacent to the SNpc, respectively, showing the correct delivery trajectory. FIG. 3D: Section of an implanted TE-NSP encased in 5% MeHA (OD: 398 μm, ID: 160 μm, length: 0.6 cm) after 2 weeks and stained for all neurons (GFP) and dopaminergic neurons (TH). Scale bars: FIG. 3A: 500 μm; FIGS. 3B and 3C: 200 μm; FIG. 3D: 250 μm.

FIGS. 4A-40: Growth, cytoarchitecture, and dopaminergic phenotype expression in human TE-NSPs encased in MeHA and fabricated with dimensions appropriate for rat implantation. FIG. 4A: Phase-contrast image of human iPSC-sourced dopaminergic neurons aggregated at ˜41 dd and cultured on a laminin-coated surface for 21 DIV. FIG. 4B: Magnification of a region in the 2D culture showing neuron dispersal and neurite outgrowth from the aggregate. FIG. 4C: Stained human dopaminergic aggregate at 24 DIV showing axons (β-tubulin III), dopaminergic neurons (TH), and nuclei (Hoechst). FIG. 4D-4F: Phase contrast imaging of the same 3% MeHA-encased human TE-NSP (OD: 398 μm, ID: 160 μm, length: 0.57 cm, seeded at ˜40 dd) at 8, 14, and 21 DIV, respectively. FIGS. 4G-41: Neurite density changes over time can be observed in magnified images of the center of the TE-NSPs in D-F. FIGS. 4J-4K: Boxplots representing the distribution of neurite growth lengths and rates at 8, 14, and 21 DIV in rat-sized human TE-NSPs (ID: 160 μm, length: ˜0.5 cm) built with 3% MeHA and made from independent cell batches seeded in the 40-43 dd range (n=3). FIGS. 4L-4M: Human TE-NSPs in 3% MeHA were stained for axons (β-tubulin III), dopaminergic neurons (TH), and nuclei (Hoechst) at 23 DIV and 57 DIV, respectively. FIGS. 4N-40: High-magnification images of the boxed regions in M showing the aggregate and axonal tract region, respectively. Scale bars: FIGS. 4A and 4C: 500 μm; FIGS. 4D-4F, FIGS. 4L and 4M: 250 μm; FIGS. 4B, 4G, 4H, 4I, 4N and 4O: 100 μm.

FIGS. 5A-5J: Human TE-NSPs fabricated with MeHA hydrogel scaled-up in length and diameter. Phase-contrast images showing human TE-NSPs in 3% MeHA micro-columns, seeded at 43 dd, with a length of ˜1 cm and OD/ID of FIGS. 5A and 5B: 700/300 μm and FIGS. 5E and 5F: 973/500 μm at 21 and 42 DIV, respectively. FIGS. 5C, 5D, 5G and 5H: Boxed regions corresponding to the central neurite regions are magnified to display growth and density changes over time. FIGS. 5I and 5J: Neurite growth lengths and rates plotted as a function of time and the ID of the micro-column. Individual data points are presented as well as the mean±SEM, with the asterisks signifying statistically significant differences relative to the value at 8 DIV for the same ID (*P<0.05, ** P<0.01, *** P<0.001 from Tukey's multiple comparisons test). Scale bars: FIGS. 5A, 5B, 5E and 5F: 500 μm; FIGS. 5C and 5D: 100 μm; FIGS. 5G and 5H: 200 μm.

FIGS. 6A-6E: Functionality of MeHA-encased human TE-NSPs in terms of evoked dopamine release and co-culture with striatal neurons. FIGS. 6A and 6B: Concentration traces and CVs showing electrically-evoked release of dopamine within the human dopaminergic aggregate in 3% MeHA TE-NSPs (ID: 160 μm, length: ˜0.5 cm) when measured from different constructs at 29 DIV (˜70 dd) and 57 DIV (˜100 dd), respectively. The time of electrical stimulation is represented by a black bar. The effect of perfusion with L-DOPA on the concentration profile is also shown. The dopaminergic identity of the released molecules was confirmed with the CV at maximum release (center) and colorplots of CVs throughout time (bottom). The characteristic signal was observed in the CVs with peak oxidation and reduction currents around 0.65 and −0.3 V, respectively. The oxidation current was also seen in the colorplots as the region near the 300 points mark in the y axis. FIGS. 6C: Phase-contrast image of a human TE-NSP (OD: 700 μm, ID: 300 μm, length: ˜1 cm) at 28 DIV (71 dd) that was seeded at 7 DIV with a rat striatal neuron aggregate at the other end of the micro-column. FIGS. 6D and 6E: Magnification of areas of the TE-NSP showing human dopaminergic axons and physical proximity to neurites emanating from the striatal target, respectively. Scale bars: FIG. 5C: 500 μm; FIGS. 5D and 5E: 250 μm.

FIGS. 7A-7M: Growth characterization and optimization in human-scale TE-NSPs. FIGS. 7A, 7E and 7I: Human dopaminergic aggregates having a low (26,550 neurons; n=3), medium (53,100 neurons; n=4), and high (106,200 neurons; n=3) number of neurons were seeded on 3% MeHA hydrogel micro-columns having an inner diameter of 500 μm and a length of ˜1 cm and imaged over time. FIGS. 7B-7D, 7F-7H and 7J-7L: Higher magnification of the boxed regions in the images in FIGS. 7A, 7E, and 7I, respectively, showing the longer growth and neurite density in the lumen of the micro-columns with aggregates having higher numbers of neurons. FIG. 7M: The neurite growth length and rate were measured over time for these human-scale TE-NSPs, with time and cell number having significant effects in both cases (p<0.0001). Data presented as mean+SEM (explain multiple comparisons).

FIGS. 8A-8E: Immunohistochemical assessment of the structure of human-scale TE-NSPs. FIGS. 8A-8C: TE-NSPs having aggregates with a low, medium, and high number of cells, respectively, were stained at # DIV to observe neurons/axons (Tuj1/b-tubulin III), dopaminergic phenotype (TH), and nuclei (Hoechst). FIGS. 8D and 8E: Zoom-in of the aggregate and axon tract regions of the construct in C to better observe the cytoarchitecture of these constructs.

FIGS. 9A-9C: Functional characterization and optimization of TE-NSPs based on electrically-evoked dopamine release. FIG. 9A: After #days, the constructs were analyzed by simultaneously stimulating and recording in the aggregate and axon tract regions, as shown in this image of a high cell number TE-NSP taken before analysis. FIG. 9B: Representative current/concentration traces and cyclic voltammograms for the three groups of constructs and both analyzed regions. Right after stimulation, the traces show a peak in concentration, and the cyclic voltammograms at the time of these peaks generally show a peak around 0.6 V, the oxidation voltage for dopamine. FIG. 9C: The quantification of evoked dopamine concentrations shows that there is significantly greater release in the aggregates in constructs with high cell numbers compared to the low and medium groups, while there are no differences in the axon tract region. Data presented as mean+SEM (* p<0.05, ** p<0.01).

FIG. 10A-10I: Recreating and characterizing the nigrostriatal pathway in vitro using TE-NSPs and a striatal aggregate. FIGS. 10A and 10B: Representative phase contrast images of a unidirectional TE-NSP having no striatal aggregate at # DIV and a bidirectional TE-NSP at # DIV having a rat striatal aggregate seeded at the end of the lumen opposite to the dopaminergic aggregate. FIGS. 10C and 10D: Higher magnification images of the boxed regions in FIGS. 10A and 10B, respectively, show neurite growth and density at the same distance from the dopaminergic aggregate. FIGS. 10E and 10F: The neurite growth lengths and rates were quantified in unidirectional and bidirectional TE-NSPs (500 μm inner diameter, 1 cm length) having a dopaminergic aggregate with low and medium cell number, respectively. Data presented as mean+SEM (stats). FIG. 10G: The functionality of bidirectional TE-NSPs and their ability to release dopamine in the striatal target was assessed with FSCV by stimulating and recording in the regions shown in the phase contrast image at # DIV range. FIG. 10H: Example of concentration traces and cyclic voltammograms in the dopaminergic aggregate, axon tracts, and axons at the striatal end of bidirectional TE-NSPs. FIG. 10I: Comparison of evoked dopamine release between unidirectional and bidirectional TE-NSPs shows no significant differences between groups. Data presented as mean+SEM.

FIGS. 11A and 11B: Cytoarchitecture of bidirectional TE-NSPs containing human dopaminergic axon tracts connecting with striatal aggregate. FIG. 11A: Confocal image showing the maximum intensity projection of a bidirectional TE-NSP, containing a human dopaminergic aggregate with a high cell number and a rat striatal aggregate, stained at # DIV for striatal neurons (DARPP-32), dopaminergic neurons (TH), and nuclei (Hoechst). FIG. 11B: A high magnification image of the striatal aggregate region shows that these neurons are physically integrated with TH+ dopaminergic axons.

FIGS. 12A-12B: The concept figures show the healthy and diseased appearance of the nigrostriatal pathway, respectively. In the healthy state, axonal fibers projected by A9 dopaminergic neurons in the substantia nigra innervate the striatum to provide the dopamine needed for proper regulation of the motor circuit. These axonal fibers spanning the nigra and striatum are known as the nigrostriatal pathway. In the parkinsonian state there is a significant loss of A9 neurons and degeneration of nigrostriatal fibers, leading to denervation of the striatum and depletion of its dopamine input.

FIGS. 13A-13C: Reconstruction of nigrostriatal fibers in the human brain. FIG. 13A: Summary of design criteria for strategies seeking to repair the axonal fibers of the nigrostriatal pathway in the human brain. FIG. 13B: Concept figure inspired by MRI and DTI studies presenting the structure of human nigrostriatal fibers and the location of areas in the substantia nigra and striatum that are relevant for the motor symptoms of PD. This image shows the regions where neurons and the axonal cytoarchitecture need to be restored to address some of the causes of PD. FIG. 13C: Tractography recreation of axonal pathways in the human brain, showing a tissue-engineered nigrostriatal pathway (TE-NSP) replacing and reconstructing the degenerated nigrostriatal pathway. TE-NSPs are constructs fabricated completely in vitro for later implantation and that feature a hydrogel encasing an aggregate of dopaminergic neurons to promote integration with native nigral inputs. The aggregate also extends axonal tracts that emulate the structure of nigrostriatal fibers and that may reinnervate the striatum to restore dopamine and thus close the loop of the motor circuit in the brain.

FIGS. 14A-14D: Rat brain response to the implantation of MeHA and agarose hydrogel columns along the nigrostriatal pathway. FIGS. 14A-14C: Acellular hydrogel micro-columns (OD: 345 μm, ID: 160 μm, length: ˜0.5 cm) fabricated with 1% agarose (n=3), 3% MeHA (n=5), and 5% MeHA (n=5), respectively, were implanted along the nigrostriatal pathway of athymic rats for 6 weeks to assess the host response. Sections orthogonal to the implant trajectory were stained for neurons (NeuN), astrocytes (GFAP), and microglia (IBA1) to determine the extent of host cell death and the inflammatory response around the micro-columns. FIG. 14D: The number of NeuN+ cells and the staining intensity of IBA1+ and GFAP+ cells in the injection side were normalized to the contralateral side and quantified in four distinct layers located at 0-50, 50-100, 100-150, and 150-200 μm from the edge of the implant, as observed in the far-right images in FIGS. 14A-14C. Two-way ANOVA tests indicated a significant effect for layer distance in the case of NeuN+ counts (p<0.0001) and for layer distance and biomaterial type in the case of IBA1 (p=0.0183, p=0.0256) and GFAP intensity (p<0.0001, p=0.0059), respectively. Data presented as mean±SEM (** p<0.01 between 3% and 5% MeHA for GFAP at the 50 μm layer). Scale bars: 100 μm.

FIGS. 15A-15G: Cytoarchitecture and growth profiles of rat-scale human TE-NSPs encased in MeHA hydrogel in vitro. FIG. 15A: Human iPSC-derived dopaminergic neurons were aggregated at 39-44 dd and cultured on a laminin-coated surface. One aggregate is shown at 24 DIV stained for axons (β-tubulin III), dopaminergic neurons (TH), and nuclei (Hoechst). FIGS. 15B, 15C: Human TE-NSPs in 3% MeHA (OD: 345 μm, ID: 160 μm, length: 5-6 mm) were stained for axons (β-tubulin III), dopaminergic neurons (TH), and nuclei (Hoechst) at 23 DIV and 57 DIV, respectively. FIGS. 15D, 15E: High-magnification images of the boxed regions in FIG. 15C showing the aggregate and axonal tracts, respectively. FIG. 15F: The neurite growth length and rates were compared at 7, 14, and 21 DIV for human TE-NSPs (n=28, 28, 14, respectively) in 3% MeHA micro-columns (OD: 345 μm, ID: 160 μm, length: 5-6 mm). One-way ANOVA indicated a significant effect for time on growth length (p<0.0001), but not for growth rate (p=0.1068). FIG. 15G: The effect of biomaterial composition on growth length and rates was characterized using one batch of human TE-NSPs made with 3% MeHA (n=14) or 5% MeHA (n=16) hydrogel micro-columns. While time did have a significant effect (p<0.0001), the biomaterial did not have a significant effect on length (p=0.5051) and rate (p=0.4056). Data presented as mean+SEM (**** p<0.0001, from Tukey's multiple comparisons test). Scale bars: FIGS. 15A: 500 μm; 15B, 15C 250 μm; 15D, 15E 100 μm.

FIGS. 16A-16B: Expression of human neural and midbrain dopaminergic phenotypes in human TE-NSPs in vitro. Human TE-NSPs encased on 3% MeHA hydrogels (OD: 345 μm, ID: 160 μm) were stained to visualize: FIG. 16A: human neurons/axons (hNCAM), dopaminergic neurons/axons (TH), and nuclei (Hoechst); FIG. 16B: A10 (calbindin) and A9 (GIRK2) dopaminergic phenotypes. Scale bars: FIGS. 16A: 250 μm; 16B: 150 μm.

FIGS. 17A-17G: Evaluation of evoked dopamine release in human TE-NSPs in vitro. FIG. 17A: Representative image of a rat-scale human TE-NSP at 28-30 DIV, showing the placement of the stimulation electrode and working (carbon fiber) electrode in the aggregate and axons. FIGS. 17B, 17C: Representative concentration traces and cyclic voltammograms at peak release for TE-NSPs encased in 3% and 5% MeHA, respectively. The black bars represent the moment of electrical stimulation. The chemical signature of dopamine in the voltammograms corresponds to a peak and trough in current at around 0.6 and −0.3 V. FIG. 17D: The dopamine release was quantified in the aggregate and axon tracts as a function of the composition of the hydrogel (3% MeHA: n=4; 5% MeHA: n=4). Two-way ANOVA suggested that the recording/stimulation region and the biomaterial do not have a significant effect on dopamine concentration (p=0.3845 and p=0.5067, respectively). FIG. 17E: We also fabricated human TE-NSPs with an aggregate of rat embryonic striatal neurons at the end opposite from the dopaminergic aggregate within a 3% MeHA micro-column with an ID of 500 μm. The phase image shows a representative construct used at 34-35 DIV to measure dopamine. FIG. 17F: Representative concentration traces and cyclic voltammograms for one bidirectional TE-NSP. FIG. 17G: Dopamine concentrations after stimulation were quantified in the dopaminergic aggregate, axon tracts, and axons in the striatal end (n=3). One-way ANOVA indicated no significant differences in concentration due to the recording/stimulation region (p=0.7746). Scale bars: FIG. 17A: 250 μm; FIG. 17E 500 μm.

FIGS. 18A and 18B: Innervation of a striatal aggregate by dopaminergic axons from a human TE-NSP in vitro. FIG. 18A: Confocal image of a scaled-up human TE-NSP (OD: 973 μm, ID: 500 μm, length: ˜1 cm) with a rat striatal aggregate at 35 DIV stained for dopaminergic neurons/axons (TH), MSNs (DARPP-32), and nuclei (Hoechst). FIG. 18B High magnification of the boxed area demonstrating dopaminergic innervation of the striatal aggregate. Scale bars: 250 μm.

FIGS. 19A-19H: Histological assessment of the survival of neurons in human TE-NSPs after implantation in a rat model of PD for 12 weeks. FIG. 19A: Confocal image of one z-plane of a thick brain section, implanted with a 3% MeHA human TE-NSP for 12 weeks, that was cleared and stained for dopaminergic neurons/axons (TH) and human neurons (hNCAM; green). FIGS. 19B, 19C: Magnification of the aggregate in two different z-planes, showing some host ingrowth (TH+/hNCAM−) into the construct and TE-NSP outgrowth (hNCAM+) into the nigral region. FIG. 19D: Magnification of the TE-NSP reconstructed from different z-planes to show the complete cytoarchitecture. FIG. 19E: Zoom-in of the axon tracts demonstrates that these are maintained 12 weeks post-implantation. FIG. 19F: Phase contrast image of the TE-NSP in FIGS. 19A-19E at 17 DIV before implantation, with the aggregate area denoted. FIG. 19G: The area of the aggregate in TE-NSPs was quantified before surgery and 12 weeks after. FIG. 19H: The average percentage of the area at 12 weeks relative to the pre-surgery area (±standard deviation) was calculated as a measure of cell preservation (n=5). The individual points are matched in FIGS. 19G and 19H to denote the same TE-NSP. Scale bars: FIGS. 19A: 500 μm; 19B, 19C: 200 μm; 19D: 250 μm; 19E: 125 μm; 19F: 100 μm.

FIGS. 20A-20N: Histological assessment of axon preservation in TE-NSPs and striatal innervation 12 weeks after implantation in rat model of PD. FIGS. 20A-20C: Thick sagittal sections containing the implanted human TE-NSPs (OD: 345 μm, ID: 160 μm, length: 5-6 mm; n=5 for histology) were cleared and stained for dopaminergic neurons/axons (TH) after 12 weeks. The images show individual z-planes spanning ˜260 μm of the thickness of a construct. FIG. 20D: Reconstruction of the full-length TE-NSP in FIGS. 20A-20C from several z-planes. FIGS. 20E-20G: Zoom-ins showing the nigral end of the construct, preserved inner axon tracts, and axons ending near the striatum, respectively. FIG. 20H: Acellular hydrogel micro-columns (same dimensions; n=3) were also implanted as comparison. The image shows a single z-plane with TH staining (red). FIG. 20I: Composite image of the full-length acellular micro-column. FIGS. 20J, 20K: Magnification of the nigral and striatal end of the acellular micro-column, respectively. FIGS. 20L, 20M: Representative z-planes for the no repair group (n=3) and the contralateral (non-lesioned) side of the brain 12 weeks after surgery, respectively, and stained for TH (red). FIG. 20N: The percentage of the area of the striatum having TH+ innervation was used as a proxy for striatal reinnervation across three ROIs: entire striatum (top), dorsal striatum (center), and striatal edge near the end of implanted constructs (bottom) (TE-NSP: n=5; acellular/no repair: n=5; contralateral: n=4). Without consideration of the contralateral side, unpaired t-tests indicated the following p-values for the effect of repair type on TH+ area: 0.3382, 0.0040, and 0.0237, for each ROI respectively. Data presented as mean+SEM (*p<0.05, ** p<0.01). Scale bars: FIGS. 20A-20D. 20H, 20L, 20M: 500 μm; 20E: 100 μm; 20F-20G: 125 μm; 20H-20K: 250 μm.

FIGS. 21A-21G: Evaluation of striatal dopamine levels in lesioned rats after TE-NSP implantation. Dopamine release in the striatum was used as a functional outcome, using sections from the lesioned, implanted side of the brain and ex vivo FSCV after 12 weeks. The images show a representative section with a TE-NSP FIG. 21A: right after sectioning, FIG. 21B: after transferring to the FSCV recording chamber, and FIG. 21C: with the stimulation and carbon fiber electrodes placed in the striatum in the area near the end of the micro-column. The micro-column and the recording/stimulation area are encircled in white. FIGS. 21D-21F: Representative concentration traces and cyclic voltammograms at peak release showing the extent of evoked dopamine release in the striatum of TE-NSP (n=3), acellular (n=2), and no repair (n=2) groups, respectively. FIG. 21G: The concentration of dopamine released in the striatum 12 weeks after TE-NSP implantation was compared to the acellular and no repair groups combined (p=0.0396, t-test). Data presented as mean±SEM (*p<0.05).

FIGS. 22A-22C: Long-distance dopaminergic axon tracts in human-scale TE-NSPs in vitro. FIG. 22A: Human TE-NSP having an aggregate with 5.3×10⁴ neurons and 1.5 cm long dopaminergic axon tracts and fixed at 85 DIV and stained for neurons/axons (β-tubulin III/Tuj1), dopaminergic neurons (TH), and nuclei (Hoechst). FIGS. 22B-22C: Higher magnification of the dopaminergic aggregate and axon tract regions, respectively. Scale bars: FIGS. 22A: 500 μm; 22B, 22C) 250 μm.

FIG. 23: Implantation of scaled-up TE-NSPs along the nigrostriatal pathway for 6 months in athymic rat model of PD. Confocal image of sagittal brain sections of an athymic rat lesioned with 6-OHDA 6 months after the implantation of a scaled-up human TE-NSP (OD: 556 μm, ID: 300 μm, length: 5-6 mm). The sections were optically cleared and immunolabeled for human (hNCAM) and dopaminergic (TH) neurons and axons. The image is the maximum intensity projection of z-planes covering the thickness of the section encompassing the implanted TE-NSP. The lower end of the construct was located at a different z-plane than the rest of the implanted TE-NSP and its associated growth. It is not visible here because its reduced staining intensity, as a result of possible antibody penetration issues, was obscured after compressing the z-planes. The striatum is on the right side of the image, while the nigral region is on the lower left side. Scale bars: 500 μm.

FIGS. 24A-24C: Preservation of long axon tracts within human TE-NSPs 6 months post-implantation in athymic rat model of PD. FIG. 24A: Here we focused on the TE-NSP from the previous figure, creating a composite image comprised of different z-planes that encompassed the axon tract region of the constructs. The tissue was stained to label our implanted human neurons and axons (hNCAM) and dopaminergic tissue (TH). FIGS. 24B, 24C: Magnification of the striatal region around the end of the TE-NSP and the cytoarchitecture of the tissue within the construct, respectively. Scare bars: FIGS. 24A: 250 μm; 24B, 24C: 125 μm.

FIGS. 25A-25B: Outgrowth from implanted scaled-up TE-NSPs into the striatum after 6 months in athymic rat model of PD. FIG. 25A: Confocal image of the continuous axon tract region of another implanted TE-NSP, stained for human neural tissue (hNCAM) and dopaminergic neurons/axons (TH). FIG. 25B: Consecutive z-planes, separated by 58.6 μm, showing the striatal end of the TE-NSP and the growth of human and dopaminergic projections into the dorsal striatum. Scale bars: 500 μm.

FIGS. 26A-26C: Survival and growth of scaled-up TE-NSPs into the region of the substantia nigra at 6 months post-implant in athymic rat model of PD. FIG. 26A: Confocal image of one z-plane, stained for TH+ neurons and axons and hNCAM+ neurons and axons showing the presence of growth from the nigral end of the implanted TE-NSPs into the host brain. FIGS. 26B, 26C: Magnification of two regions of this TE-NSP-derived showing the tissue morphology. Scale bars: FIG. 26A: 500 μm; FIGS. 26B, 26C: 125 μm.

FIGS. 27A-27D: Evoked dopamine release in the striatum and within implanted TE-NSPs after 6 months in athymic rat model of PD. We show representative results, including concentration traces (left) and cyclic voltammograms at peak release (center) and over time (right), from ex vivo FSCV measurements of the concentration of dopamine released after electrical stimulation (black bar) in: FIG. 27A the inner edge of the dorsal striatum along the trajectory of an implanted TE-NSP; FIG. 27B: within the dorsal striatum in the direction of the implant; FIG. 27C: within the aggregate end of the implanted TE-NSP; FIG. 27D: within the axon tract region of the TE-NSP. In all cases both stimulation and recording were performed in the same region.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

As used herein, the term “biofabricated” refers to the process of creating, building, and/or assembling as related to biological, biocompatible, and/or biologically inspired components

As used herein, the term “cylinder” or “cylindrical” includes a surface consisting of each of the straight lines that are parallel to a given straight line and pass through a given curve. In some embodiments, cylinders have an annular profile. In other embodiments, the cylinder has a cross-section selected from the group consisting of: a square, a rectangle, a triangle, an oval, a polygon, a parallelogram, a rhombus, an annulus, a crescent, a semicircle, an ellipse, a super ellipse, a deltoid, and the like. In other embodiments, the cylinder is the starting point of a more complex three-dimensional structure that can include, for example, complex involutions, spirals, branching patterns, multiple tubular conduits, and any number of geometries that can be implemented in computer-aided design, 3-D printing, and/or in directed evolutionary approaches of secretory organisms (e.g., coral), including of various fractal orders.

As used herein, the term “micro-tissue” means a three-dimensional construct containing some combination of cells and biomaterials that has at least one dimension measuring hundreds to thousands of microns.

As used herein, the term “modified hyaluronic acid” means hyaluronic acid that has been chemically modified to at least a fraction of the disaccharide repeat units, such as, without limitation, to allow for crosslinking of the hyaluronic acid into a hydrogel.

As used herein, “synapse” refers to a junction between a neuron and another cell, across which chemical communication flows.

As used herein, “synapsed” refers to a neuron that has formed one or more synapses with one or more cells, such as another neuron or a muscle cell.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION

### Pre-Formed Neural Networks

In one aspect, the present disclosure provides a construct comprising a pre-formed neural network, the construct comprising a micro-column comprising an outer sheath comprising a hyaluronic acid (HA) hydrogel, and a core comprising an extracellular matrix (ECM); a plurality of neurons within the micro-column. In various embodiments, the construct is biocompatible. In various embodiments, the construct is an implantable construct. In various embodiments, the hydrogel sheath is cylindrical or substantially cylindrical. In various embodiments, the ECM core substantially fills a lumen of the hydrogel sheath. In various embodiments, the micro-column is directed along a substantially straight line along its length. In various embodiments, the micro-column is directed along a curved path along its length.

In various embodiments, the plurality of neurons have cell bodies substantially localized in proximity to a first end of the micro-column and extend axons longitudinally along at least a portion of a length of the micro-column. In various embodiments, the plurality of neurons comprise one or more three dimensional aggregates. In various embodiments, the axons are located within and extend longitudinally along a lumen of the hydrogel sheath from the neurons at the first end and towards the opposite end, extending in some embodiments along the entire length of the construct and terminating at the end opposite the first end and in other embodiments extending a portion of the distance between the first end and the opposite end. In various embodiments, the axons of the plurality of neurons extend along at least 50% of the length of the micro-column, extend along at least 75% of the length of the micro-column, or extend along 90% of the length of the micro-column. In various embodiments, the axons grow through the ECM of the core and/or along an interface between an inner surface of the hydrogel sheath and the ECM of the core. As described further in Examples 1 and 2, in some embodiments the inner surface of the hydrogel sheath provides a substrate that facilitates axon extension through the ECM lumen. In various embodiments, the neuronal cells and axons extending therefrom have a cyto-architecture that replicates long-range axon tracts present in a subject. In various embodiments, the subject is a mammal. In various embodiments, the subject is a human subject. In various embodiments, the neuronal cells and axons extending therefrom have a cyto-architecture that replicates long-range axon tracts present in a brain of a human subject. In various embodiments, the neuronal cells and axons extending therefrom have a cyto-architecture that mimics a native axon pathway between the substantia nigra and the striatum in a brain of a subject. In various embodiments, the axon tracts are or have been pre-directed via the micro-column. In various embodiments, the neurons together with the axons extending therefrom form a biofabricated micro-tissue.

In various embodiments, the outer diameter of the micro-column ranges from about 500 microns to about 2,500 microns. In various embodiments, the outer diameter of the micro-column ranges from about 500 to about 1,500 microns. In various embodiments, the outer diameter of the micro-column ranges from about 750 to about 1,000 microns. In various embodiments, the outer diameter is a cross-sectional diameter of the hydrogel sheath. In various embodiments, the outer diameter is a cross-sectional diameter of the hydrogel sheath, including any outer coatings thereon. In various embodiments, the inner diameter of the micro-column ranges from about 250 microns to about 2,000 microns. In various embodiments, the inner diameter of the micro-column ranges from about 250 to about 1,000 microns. In various embodiments, the inner diameter of the micro-column is about 500 microns. In various embodiments, the ECM comprises a polysaccharide. In various embodiments, the ECM comprises one or more members selected from the group consisting of collagen, fibrin, fibronectin, gelatin, hyaluronic acid, laminin, and Matrigel. In various embodiments, the ECM comprises collagen. In various embodiments, the ECM comprises collagen at a concentration ranging from about 0.1 to 10 mg/ml. In various embodiments, the ECM comprises collagen at a concentration about 1 mg/ml. In various embodiments, the ECM comprises laminin. In various embodiments, the ECM comprises laminin at a concentration ranging from about 0.1 to 10 mg/ml. In various embodiments, the ECM comprises laminin at a concentration of about 1 mg/ml.

### Hyaluronic Acid Hydrogels

In various embodiments, the hyaluronic acid (HA) hydrogel present in various embodiments of the construct is or comprises a modified hyaluronic acid (e.g., a cross-linked modified hyaluronic acid). In various embodiments, the modified hyaluronic acid is methacrylated HA (MeHA). In various embodiments, the hyaluronic acid is or comprises about 0.5 to about 20% wt MeHA. In various embodiments, the hyaluronic acid is or comprises 3-5% MeHA. In various embodiments, the modified HA comprises one or more members selected from the group consisting of norbornene-modified HA, acrylated HA, maleimide HA, and hydroxyethyl methacrylate HA. In various embodiments, the outer hydrogel sheath is a 3D printed and photopolymerized MeHA cylinder.

MeHA hydrogels may be further optimized to have the combination of molecular weight, macromer concentration, degree of methacrylation, and light irradiation time to yield the desired mechanical properties. The molecular weight of HA can be selected before the methacrylation process by using the HA with the desired weight as one of the reactants. In various embodiments, the HA acid has a molecular weight in the range between about 50 kDa and about 1.5 MDa. In various embodiments, the HA has a molecular weight of 50 kDa, 60 kDa, 70 kDa, 80 kDa, 90 kDa, 1 MDa, 1.1 MDa, 1.2 MDa, 1.3 MDa, 1.4 MDa, or 1.5 MDa.

The degree of methacrylation can be changed by decreasing or increasing the duration of the reaction between HA and methacrylic anhydride. In various embodiments, the degree of HA methacrylation is about 40%. In various embodiments, the degree of HA methacrylation is between about 25% and 100%.

The macromer concentration refers to the percentage of MeHA in the hydrogel solution that is created before photopolymerization. In various embodiments, the concentration of MeHA is between about 1% and about 10%. In various embodiments, the concentration of MeHA is between about 3% and about 5%.

In some embodiments, properties of the hydrogel change as a function of time while exposed to the ultraviolet light. Therefore, in various embodiments, the exposure time is varied to modulate the properties of the hydrogel. In various embodiments, the hydrogel is exposed to ultraviolet light until all available crosslinks are formed. In various embodiments, the exposure time is about 5 min. In various embodiments, the exposure time is about 1 minute, about 2 minutes, about 3 minutes or about 4 minutes. Overall, higher molecular weights, degrees of methacrylation, macromer concentrations, and light irradiation times result in MeHA hydrogels that are stiffer and slower to degrade in the presence of hyaluronidases. In addition to MeHA, other types of modified HA can be used to confer other crosslinking behaviors and degradation properties. For instance, HA hydrogels can be made with increased degradation through hydrolysis or through matrix metalloproteinase degradation with peptide crosslinkers. Varying different types of crosslinkers within a hydrogel can also change properties. MeHA hydrogels crosslinked by radical photopolymerization are mainly degradable by enzymes known as hyaluronidases. When implanted, the environment in which the hydrogel is found may not have the hyaluronidases to the extent required for meaningful degradation to occur. Other types of modified HA may be used to connect the HA chains with modified methacrylates or other chemical groups to make the hydrogel susceptible to ester hydrolysis or with enzymatically degradable crosslinker groups. These two would allow for significant degradation to occur, which can be tuned similarly as explained above by tuning the crosslinking density of the hydrogel to get slower or faster degradation. In various embodiments, the outer hydrogel sheath comprises one or more hydrolysis sensitive compounds within the crosslinks. In various embodiments, the one or more hydrolysis sensitive compounds comprise esters such as lactic acid, caprolactone or anhydrides. In various embodiments, hydrolysis sensitive moieties are incorporated into the crosslinks. In various embodiments, at least about 50%, at least about 60%, at least about 70%, at least about 90%, at least about 95% of the crosslinks comprise hydrolysis sensitive moieties. In various embodiments, the outer hydrogel sheath comprises methacrylated hyaluronic acid doped with the one or more hydrolysis sensitive compounds. In various embodiments, the hydrolysis sensitive compounds are located between HA and the methacrylate groups. In various embodiments, the outer hydrogel sheath comprises one or more di-thiol peptides.

In various embodiments, the constructs present various biochemical cues to the host. In various embodiments, an addition reaction (non-light based) is performed to consume some but not all methacrylates of MeHA. In various embodiments, subsequent exposure to light consumes the remaining methacrylates and crosslinks the gel.

In various embodiments, the HA is norbornene-HA. In various embodiments, a protein or other thiol containing molecule is cross-linked to norbornene-HA by exposure to light. In various embodiments, the protein or other thiol containing molecule is linked to some but not all norbornenes. These molecules would be covalently bound to the HA backbone through the norbornene. In various embodiments, a crosslinker having di-thiols to crosslink norbornenes and form the hydrogel. NorHA is added. In various embodiments, at least a portion of the one or more di-thiol peptides are sensitive to cleavage. In various embodiments, at least a portion of the one or di-thiol peptides are sensitive to cleavage by matrix metalloproteinases expressed by cells. In various embodiments, the modified HA is maleimide or acrylate functionalized hyaluronic acid.

In various embodiments, growth factors and drugs can be loaded into the hydrogel sheath by mixing with the hydrogel solution during fabrication, passively by swelling the hydrogel in media containing those molecules, or by covalently binding those molecules to the HA chains. In this case, the molecules may be released during the normal degradation process of the hydrogel, with faster degradation rates leading to faster release. As explained above, there are several ways to tune the degradation rate of the HA in the construct and to make it susceptible to hyaluronidases, hydrolysis, and/or the action of enzymes released by cells. In other instances, modified HA has been covalently bound to structures containing drugs like dexamethasone that can be released later on in response to a trigger such as mechanical stress. HA has been investigated for light-triggered drug release, where nanoparticles release drugs after irradiation with near-infrared light leads to hydrolysis of susceptible bonds in modified HA. Modified HA has also been covalently bound to heparin to allow for the retention and slow release of growth factors that have heparin-binding domains. In this case, thiolated heparin can be bound to the chemical groups added to HA, and the release profile can be tuned according to the concentration of heparin and its molecular weight.

### Types of Neurons

In various embodiments, the plurality of neurons comprises dopaminergic neurons. In various embodiments, at least 10%, at least 20%, at least 30%, at least 40%, at least 50%, at least 60%, at least 70%, at least 80% or at least 90% of the plurality of neurons are dopaminergic neurons. In various embodiments, the dopaminergic neurons are obtained via microdissection. In various embodiments, the dopaminergic neurons are obtained via differentiation from stem cells. In various embodiments, the dopaminergic neurons are obtained via cell sorting or purification. In various embodiments, the dopaminergic neurons comprise midbrain dopaminergic neurons. In various embodiments, the midbrain dopaminergic neurons comprise A9 neurons.

In various embodiments, the plurality of neurons comprise GABAergic neurons. In various embodiments, at least 10%, at least 20%, at least 30%, at least 40%, at least 50%, at least 60%, at least 70%, at least 80% or at least 90% are GABAergic neurons. In various embodiments, the GABAergic neurons are obtained via microdissection. In various embodiments, the GABAergic neurons are obtained via differentiation from stem cells. In various embodiments, the GABAergic neurons are obtained via cell sorting or purification.

In various embodiments, the plurality of neurons comprise glutaminergic neurons. In various embodiments, at least 10%, at least 20%, at least 30%, at least 40%, at least 50%, at least 60%, at least 70%, at least 80% or at least 90% of the plurality of neurons are glutaminergic neurons. In various embodiments, the glutaminergic neurons are obtained via microdissection. In various embodiments, the glutaminergic neurons are obtained via differentiation from stem cells. In various embodiments, the glutaminergic neurons are obtained via cell sorting or purification.

In various embodiments, the plurality of neurons comprise cholinergic neurons. In various embodiments, at least 10%, at least 20%, at least 30%, at least 40%, at least 50%, at least 60%, at least 70%, at least 80% or at least 90% of the plurality of neurons are cholinergic neurons. In various embodiments, the cholinergic neurons are obtained via microdissection. In various embodiments, the cholinergic neurons are obtained via differentiation from stem cells. In various embodiments, the cholinergic neurons are obtained via cell sorting or purification. In various embodiments, the plurality of neurons, whether dopaminergic, GABAergic, glutaminergic or cholinergic, comprise midbrain neurons.

### Human Scale Constructs

In various embodiments, the plurality of neurons comprise human neurons. In various embodiments, the human neurons comprise induced pluripotent stem cell (iPSC)-derived neurons. In various embodiments, the plurality of neurons comprise human A9 dopaminergic neurons. In various embodiments, the human A9 dopaminergic neurons comprise stem cell-derived human A9 dopaminergic neurons. The construct of any one of the preceding claims, wherein the neurons comprise A9 dopaminergic neurons and exhibit dopamine release of at least 50 nM as measured via fast scan cyclic voltammetry. In various embodiments, the plurality of neurons releases and/or has a quantity of dopaminergic neurons sufficient to release dopamine at a level sufficient to provide for a level of at least 4 ng/mg in tissue. In various embodiments, the dopaminergic neurons release dopamine at a level sufficient to provide for a level of at least 4 ng/mg in tissue within two weeks, within four weeks or within six weeks after implantation in a subject. In various embodiments, the plurality of neurons provides for an increase in 18F-DOPA uptake in a putamen of a subject at a level of about 50-60% or of about 80-100% of a normal value, in various embodiments within about 4, about 6 or about 8 weeks following implantation.

In various embodiments, neurons of constructs described herein provide for increase in 18F-DOPA uptake in the putamen of the subject at the level of about 50-60% of a normal value upon implantation in the subject.

In various embodiments, the stem cells from which the A9 dopaminergic neurons are derived are induced pluripotent stem cells (iPSCs). In various embodiments, the plurality of neurons comprises at least 50,000 neurons. In various embodiments, the plurality of neurons comprises at least 100,000 neurons. In various embodiments, the plurality of neurons comprises at least 125,000 neurons. In various embodiments, the plurality of neurons comprises at least 500,000 neurons. In various embodiments, the plurality of neurons comprises at least 1,000,000 neurons.

In various embodiments, the micro-column has a length ranging from about 2 to about 5 centimeters. In various embodiments, the axons of the plurality of neurons extend a distance ranging from about 2 to about 5 centimeters along the length of the micro-column. In various embodiments, the micro-column is suitable for implantation along a trajectory encompassing a substantia nigra (SN) region and a striatum region of a subject. In various embodiments, the SN region is a ventrolateral SN region. In various embodiments, the striatum region is a dorsal striatum region. In various embodiments, the subject is a mammal. In various embodiments, the subject is a human subject.

### Method of Manufacture

In another aspect, the present disclosure provides a method of manufacturing a construct comprising a pre-formed neural network, the method comprising: seeding a first end of a micro-column with a plurality of neural precursor cells and/or neurons selected from the group consisting of dopaminergic, GABAergic, glutaminergic or cholinergic neuronal precursors or neurons; and culturing the micro-column and plurality of neural cells seeded therein in-vitro. In various embodiments, the construct is a biocompatible construct. In various embodiments, the construct is an implantable construct. In various embodiments, the construct is for use in an in-vitro test bed. In various embodiments, the step of culturing the micro-column and plurality of neural cells seeded therein in-vitro comprises causing growth of axons from the neural cells, along a length of the micro-column, toward a second, opposite, end of the micro-column.

In various embodiments, the method further comprises determining that axon growth from the plurality of neural cells has reached a particular length; and responsive to the particular length of axon growth being determined to have been reached, packaging (e.g., in a sterile package) and/or providing the micro-column for implantation. In various embodiments, the particular length is a predetermined desired length. In various embodiments, the particular length ranges from about 2 to about 5 centimeters. In various embodiments, the step of determining that axon growth from the plurality of neural cells has reached a particular length comprises imaging the micro-columns and neural cells therein. In various embodiments, determining that axon growth from the plurality of neural cells has reached a particular length comprises imaging via fluorescence or other microscopy, fast-scan cyclic voltammetry (FSCV), staining, sectioning, or determining axon density. In various embodiments, the plurality of neural cells with which the micro-column is seeded comprises neural cell aggregates. In various embodiments, the neural cell aggregates comprise a plurality of approximately spherical aggregates of neural cells. In various embodiments, each neural cell aggregate comprises cells at a density ranging from about 100,000 to about 300,000 neurons per aggregate. In various embodiments, a plurality of the neural cell aggregates exhibit a diameter of at least 500 μm. In various embodiments, the micro-column comprises a hydrogel sheath and a core comprising an extracellular matrix (ECM), and wherein the neural cells are seeded to be in direct contact with the ECM of the core. In various embodiments, the hydrogel sheath comprises MeHA. In various embodiments, the hydrogel sheath of the micro-column is a 3D printed cylinder. In various embodiments, the method comprises, 3D printing the hydrogel sheath prior to seeding the first end of the micro-column. In various embodiments, the method comprises differentiating human induced pluripotent stem cells (iPSCs) for a particular differentiation period to produce differentiated cells and, following differentiating the iPSCs for the particular differentiation period, and seeding the micro-column using the differentiated cells as the neural cells. In various embodiments, the method comprises seeding the differentiated iPSCs in the micro column after about 40 dd. In various embodiments, the method comprises seeding the differentiated iPSCs in the micro-column after about 11 to about 20 dd. In various embodiments, the method comprises seeding the differentiated iPSCs in the micro-column once dopaminergic precursor fate is established and when the cells are usually replanted and matured further.

### In-Vitro Test Bed

In various embodiments, the present disclosure provides an in vitro test bed comprising the construct as described herein, comprising a first population of neurons and axons grown therefrom; and a second population of neurons, synapsed with the first population. In various embodiments, the second population of neurons comprises or are striatal neurons. In various embodiments, the second population of neurons are seeded at an end of the construct opposite to the end at which the first population of neurons were seeded. In various embodiments, axons from the first population of neurons extend longitudinally from a first end of the construct along a length of the construct and synapse with the second population seeded at a second, opposite, end of the construct. In various embodiments, cell bodies of the first population are localized in substantial proximity to the first end of the construct.

### Methods of Treatment

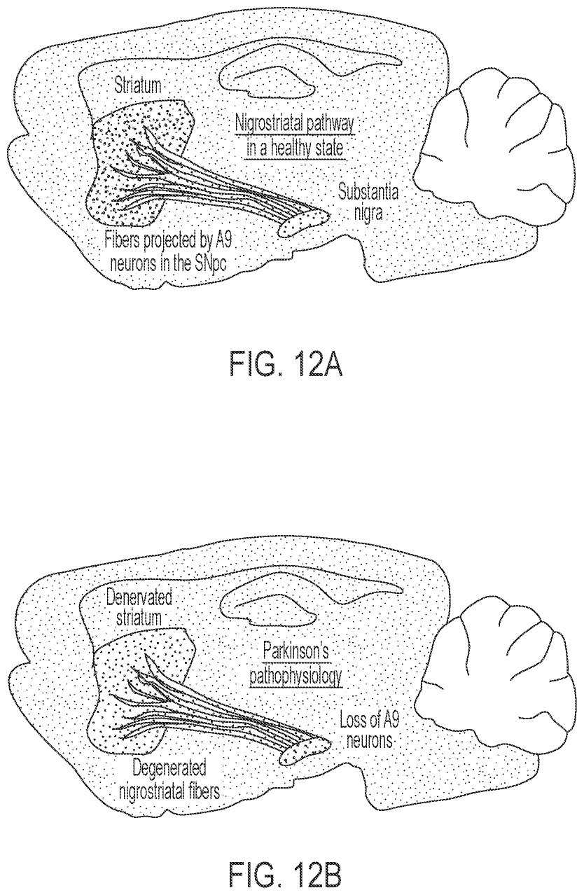

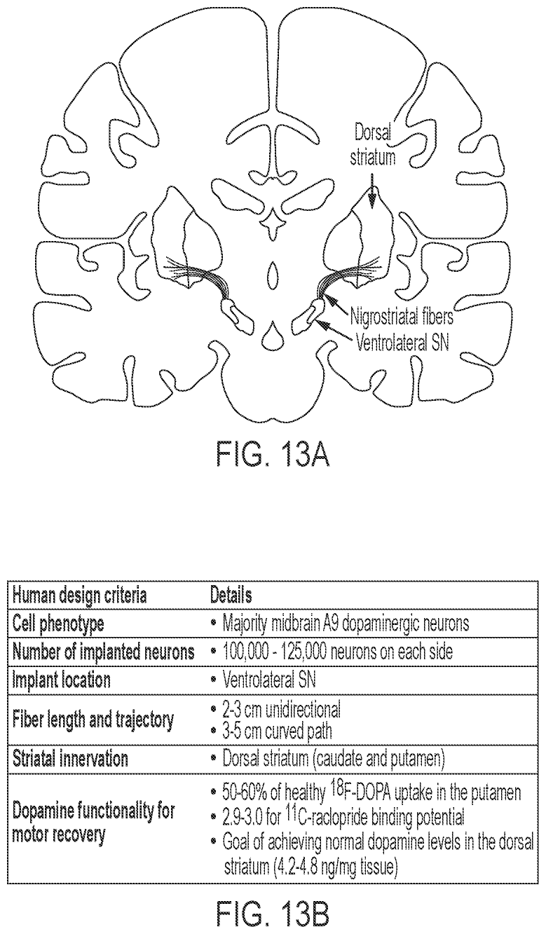

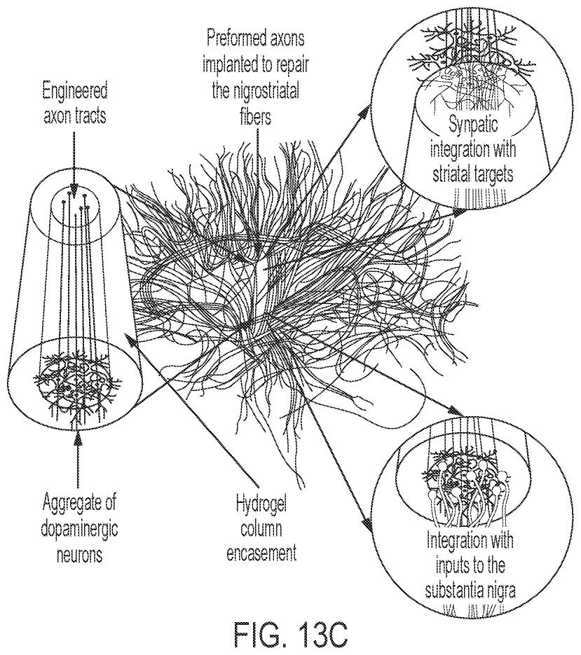

In various embodiments, the present disclosure provides methods of at least partially replacing a population of neurons forming a pathway between the substantia nigra and striatum in a subject, the method comprising implanting at least one construct as described herein into a brain of the subject. Without meaning to be limited by theory, as illustrated in FIGS. 12A-12D and 13A-13C, implantation of constructs as described herein into the nervous system of a subject treats various conditions associated with neural degeneration or damage, by way of non-limiting example, Parkinson's disease. Accordingly, in various embodiments, the method comprises ameliorating one or more conditions of the subject. In various embodiments, ameliorating the one or more conditions comprises restoring motor function of the subject. In various embodiments, ameliorating the one or more conditions comprises reducing pain of the subject. In various embodiments, ameliorating the one or more conditions comprises reducing tremors of the subject. In various embodiments, the method comprises implanting the at least a portion of one construct within a substantia nigra of the subject. In various embodiments, following implantation, the neurons of the construct synapse with host neurons in a brain of the subject. In various embodiments, the host neurons with which the neurons of the construct synapse comprise, medium spiny neurons (MSNs) in a dorsolateral striatum of the subject. In various embodiments, the subject is a human subject. In various embodiments, implanting the at least one construct comprises using MRI-guided neurosurgery. In various embodiments, implanting the at least one construct comprises implanting a plurality of constructs. In various embodiments, implanting the plurality of constructs comprises implanting a plurality of constructs in a single hemisphere of the brain of the subject. In various embodiments, implanting the plurality of constructs comprises implanting one or more constructs in each hemisphere of the brain of the subject. In various embodiments, 1 to 3 constructs are implanted in each hemisphere of the brain of the subject. In various embodiments, implantation in a first hemisphere is performed via a first surgery and implantation in a second hemisphere is performed via a second surgery, performed at a different time than the first surgery. In various embodiments, the second surgery is performed about 6 months after the first surgery.

### EXAMPLES

### Example 1

### Materials and Methods:

### MeHA Synthesis and Mechanical Characterization

MeHA was synthesized by the esterification of hyaluronic acid (Lifecore, molecular weight) and methacrylic anhydride (Sigma, 276685) in deionized water for ˜3.5 hr at pH 8.5 for a ˜40% degree of methacrylation. The reaction was purified by dialysis for 5-7 days, and the product was recovered by lyophilization. The degree of modification was assessed from the integration of the area below the two vinyl singlets at ˜5.8 and ˜6.3 ppm relative to the area representative of the sugar ring in 1H-NMR spectra of lyophilized MeHA. To study gelation kinetics, MeHA solutions were prepared in Dulbecco's phosphate buffered saline (DPBS; ThermoFisher, 14190136) with 0.05% weight/volume (w/v) Irgacure 2959 (12959; Sigma, 410896) photoinitiator. The solution was transferred between the fixed plate and the geometry of a controlled-stress rheometer (TA Instruments, AR2000) fitted to an ultraviolet (UV) lamp (Excelitas Technologies, OmniCure S1500). Using a time sweep with 0.5% strain and 1 Hz oscillations, the storage and loss moduli were recorded before and after exposure to 10 mW/cm2 UV light at 2 min to observe crosslinking in situ. The data presented are representative examples after taking at least duplicate profiles for each hydrogel. Hydrogels were also cast in ˜4.78 mm cylinders (n=5), and the compressive moduli were calculated from the slope of the stress vs. strain curve (10-20% region) using a dynamic mechanical analyzer (TA Instruments, Q800) at a 0.2 N/min loading rate.

### Fabrication of Hydrogel Micro-Columns

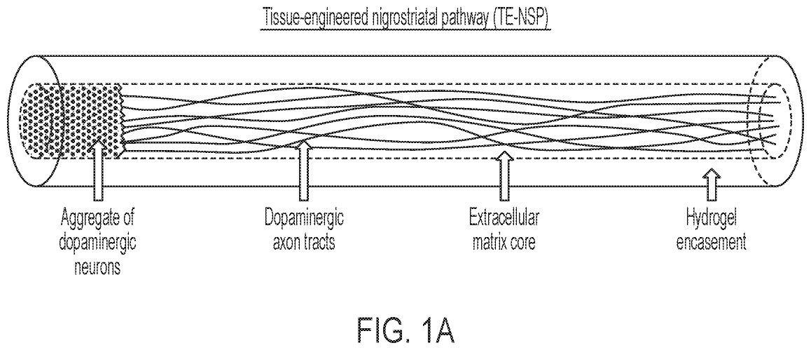

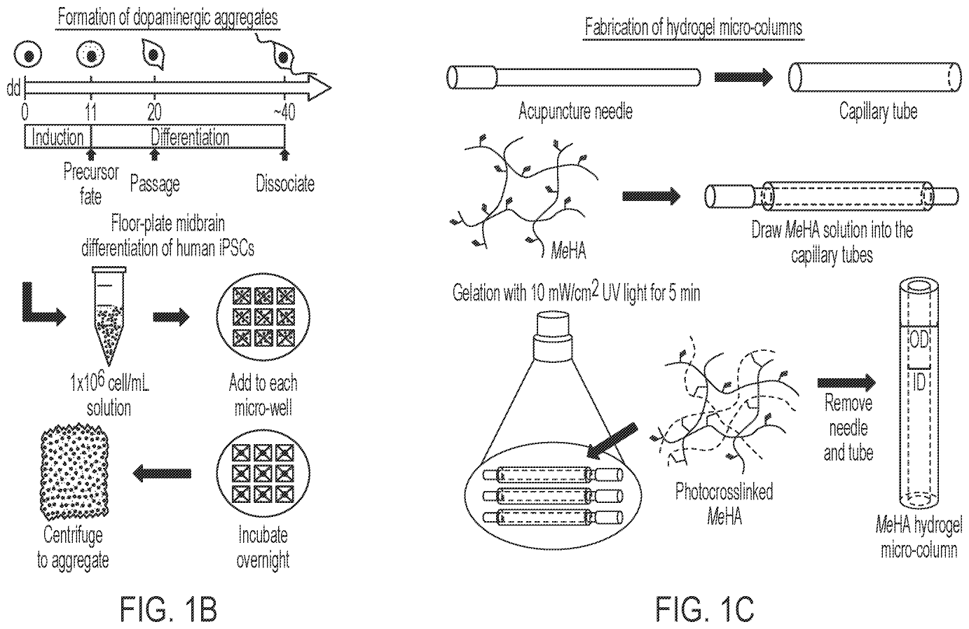

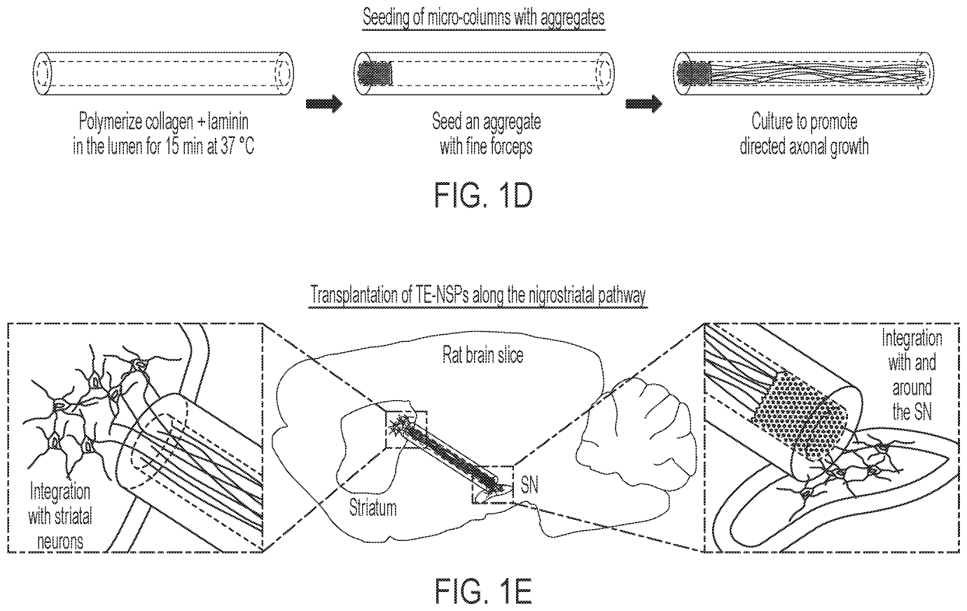

MeHA micro-columns were created by drawing up MeHA solution (with 0.05% 12959 in DPBS) by capillary action into glass capillary tubes (Drummond Scientific; inner diameters: 398, 701, 973 μm) containing an inserted acupuncture needle (Lhasa OMS; outer diameters: 160, 300, 500 μm, respectively). The needle created the inner diameter (ID) or lumen of the micro-column while the remaining space formed the outer diameter (OD) or shell. The solution within each tube was then gelled with 10 mW/cm2 UV for 5 min, a time sufficient to obtain plateaued mechanical properties. The acupuncture needles were manually pulled from the tubes, leaving the gelled micro-columns inside. The hydrogels were removed by pushing them out of the tubes and into DPBS using 20, 23, or 30 gauge needles (BD, 305178, 305120, 305128), depending on the diameter of the tubes (FIG. 1B). The micro-columns were sterilized with UV light for 30 min and cut to the desired length. For agarose micro-columns the same procedure was followed, except that agarose solutions were made by heating and stirring agarose (Sigma, A9539) in DPBS and then gelled by cooling. The final step before cell seeding consisted in filling the lumen with ECM, which was prepared with 1 mg/mL rat tail type I collagen (Corning; 354236) and 1 mg/mL mouse laminin (Corning; 354232) in Neurobasal (ThermoFisher, 21103049) at 7.2-7.4 pH. After suctioning the DPBS from the surroundings and interior of each micro-column, ECM solution was added into the lumen in excess of the volume required based on its dimensions and allowed to polymerize for 15 min at 37° C. Fresh culture media was added to the dishes containing the columns, the excess ECM adhered to the sides of the columns was removed with forceps, and the micro-columns were incubated at 37° C. until seeding.

### Neuronal Cell Culture, Aggregation, and Seeding

Rat dopaminergic neurons were isolated from embryonic day 14 (E14) rat pups as previously published. Pregnant Sprague Dawley rats (Charles River) were euthanized with carbon dioxide and decapitation, the pups were extracted, and the ventral midbrain was dissected from the brains in Hank's balanced salt solution (HBSS; ThermoFisher, 14175079). The tissue was rinsed, dissociated with accutase (ThermoFisher, A1110501) for 10 min at 37° C., and triturated with a pipette. After centrifugation at 200 g for 5 min, the cells were resuspended at 1-2×10⁶ cells/mL using media with Neurobasal, 2% B27 (Invitrogen, 12587010), 1% fetal bovine serum (FBS; Sigma, F0926), 2 mM Glutamax (ThermoFisher, 35050061), 0.3% penicillin-streptomycin (ThermoFisher, 15140122), 4 ng/mL mouse recombinant basic fibroblast growth factor (bFGF; Fisher PMG0034), and 100 μM ascorbic acid (Sigma, A5960). Cell solutions with rat embryonic striatal neurons were obtained similarly by isolating and dissociating the striatum of E18 rat pups and cultured in the same media. In some cases, the rat ventral midbrain neurons were transduced virally to express green fluorescent protein (GFP) to facilitate visualization in vivo. In these cases, rat TE-NSPs were incubated overnight at 5 DIV with media including 1/2000 of pAAV1.hSyn.eGFP.WPRE.bHG (Addgene, 105539-AAV1), with a full media change occurring on the next day.

The C1.2 human iPSC line (passages 22-35) was derived from the commercially available BJ line (ATCC® CRL-2522™). Stem cells were differentiated based on a floor-plate midbrain dopaminergic induction protocol. The iPSCs were maintained on CF-1 mouse embryonic fibroblast feeder cells (Molecular Transfer, A34181) and cultured in embryonic stem cell (ESC) media containing DMEM/F12 (ThermoFisher, 11330032), 20% knockout serum replacement (KSR; ThermoFisher, 10828010), 1 mM non-essential amino acids (ThermoFisher, 11140076), 2 mM Glutamax, 0.1 mM β-mercaptoethanol (ThermoFisher, 21985-023), 1% penicillin-streptomycin, and 6 ng/mL bFGF (R&D Systems, 233-FB-CF). Human iPSCs were then detached with accutase and cultured with ESC media on dishes coated with 1/20 Matrigel (Corning, 354234) in DMEM/F12. When confluent, the cells were exposed to 100 nM LDN193180 (ReprocellUSA, 04-0074) and 10 μM SB431542 (ReprocellUSA, 04-0010-10) in DMEM/F12, 15% KSR, 2 mM Glutamax, and 10 UM β-mercaptoethanol (KSR media). These two compounds were used for dual SMAD inhibition to induce differentiation into neuroectoderm. Then at 1 dd the media was supplemented with 100 ng/mL sonic hedgehog (SHH; R&D Systems, 464SH025CF) and 2 μM purmorphamine (ReprocellUSA, 04-0009). At 3 dd, the media also included 3 μM CHIR99021 (ReprocellUSA, 04-0004), an activator of Wnt signaling required for coexpression of the floor- and roof-plate markers FOXA2 and LMX1A, respectively. Starting on 5 dd, the media was changed to 75% KSR media and 25% N2 media, with the proportion of N2 media increasing by 25% every two days. N2 media consisted of DMEM/F12 with L-glutamine and HEPES (ThermoFisher, 11330-032), 55 μM β-mercaptoethanol, 23.8 mM sodium bicarbonate (Sigma, S5761), 0.156% w/v D-[+]-glucose (Sigma, G7021), 22 nM progesterone (SIgma, P8783), and 1% N2 Supplement (Stem Cell Technologies, 07156). The dopaminergic precursor fate was established at 11 dd after which differentiation media was added, consisting of Neurobasal with 2% B27, 2 mM Glutamax, 200 μM ascorbic acid, 20 ng/mL GDNF (Fisher Scientific, 212GD050CF), 20 ng/ml BDNF (Peprotech, 450-02), 1 ng/mL TGFβ3 (R&D Systems, 243B3002CF), 500 μM dibutyryl cAMP (Sigma, D0260), and 10 μM DAPT (Fisher, 263410). After confirming a bipolar morphology and tight packing at 20 dd, the cells were lifted using accutase and cultured on dishes coated with 15 μg/mL poly-L-ornithine (Sigma, P4957) and 5 μg/cm2 laminin. At ˜40 dd the cells were detached with accutase and resuspended in differentiation media at 1×10⁶ cells/mL for subsequent aggregation.

For micro-column seeding, dissociated dopaminergic or striatal neurons were first aggregated by adding 15 μL to each micro-well in a custom-made array of 3×3 inverted pyramidal micro-wells fitting in 12-well plates. The cells formed aggregates at the bottom after centrifugation at 1,500 rpm for 5 min and were subsequently incubated overnight (FIG. 1C). The aggregates were then pipetted out and cut and inserted with fine forceps to fit within one end of the lumen of the micro-columns (FIG. 1D). After seeding, the micro-columns had approximately 2,000-3,000 or 8,000-10,000 cells per aggregate for an ID of 160 and 500 μm, respectively. These seeded micro-columns were cultured at 37° C. and 5% CO₂, with media changes every 2-3 days. Where noted, a striatal aggregate was inserted at the other (free) end of the micro-column around 7 DIV.

### Growth Characterization of TE-NSPs

The effect of biomaterial encasement on neurite growth length and rate in rat TE-NSPs (OD: 398 μm; ID: 160 μm; length: ˜5 mm) was evaluated at 1, 3, 7, and 10 DIV for 1% agarose (n=7), 3% MeHA (n=13), and 5% MeHA (n=14). Human TE-NSPs with those dimensions were similarly characterized at 8, 14, and 21 DIV for 3% MeHA (n=10, 4, 2 for three independent runs, respectively). Scaled-up human constructs in 3% MeHA were imaged for growth measurements at 8, 14, 21, 28, and 42 DIV (length: ˜1 cm; n=4, 3 for OD/ID combinations of 700/300 and 973/500, respectively). The neurite growth lengths were measured from phase contrast images as the distance between the inner edge of the aggregate and the tip of the longest observable neurite using Fiji. The growth rates were estimated with the backward difference method using the neurite lengths at the current and previous timepoints. The images were obtained using a Nikon Eclipse Ti-S microscope and QiClick camera integrated with NIS Elements software (Nikon). Where applicable, the results were analyzed using repeated measures two-way ANOVA and post-hoc Tukey's multiple comparison test for differences between groups, with P<0.05 for statistical significance.

### In Vitro Phenotypic and Structural Characterization of TE-NSPs

The cytoarchitecture of TE-NSPs and the presence of neuronal and dopaminergic phenotypes were verified by immunolabeling and confocal imaging. TE-NSPs and other cultures were stained at several time points: 14 DIV for rat TE-NSPs; around 21, 30, and 60 DIV for human TE-NSPs. Cultures were fixed in 4% paraformaldehyde (Electron Microscopy Sciences, 15710) in DPBS for 35 min. Whole constructs were permeabilized for 1 hr with 0.3% Triton X-100 (Sigma, T8787) in 4% horse serum (ThermoFisher, 16050122) and then incubated overnight with primary antibodies in 4% horse serum at 4° C. The primary antibodies used in this study included tyrosine hydroxylase (TH; 1/500, sheep, Abcam, ab113; 1/500, rabbit, Pel-Freez Biologicals, P40101), the enzyme in the rate-limiting reaction of dopamine synthesis, and β-tubulin III ( 1/500, mouse, Sigma, T8578), a neuronal microtubule protein. Then, the cultures were exposed to appropriate fluorescent secondary antibodies ( 1/500, ThermoFisher, Alexa-488, Alexa-568, Alexa-647) in 4% horse serum for 2 hr at room temperature. Finally, the constructs were incubated for 10 min with Hoechst ( 1/10000, ThermoFisher, 33342), to stain the nuclei, and rinsed thoroughly. Immunolabeled cultures were imaged with a Nikon A1RSI laser scanning confocal microscope. Except where noted, all images used for analysis or included in this manuscript represent the maximum intensity projection of the full-thickness z-stacks.

In Vitro Functional Characterization with Fast Scan Cyclic Voltammetry

TE-NSPs were assessed for functionality by measuring electrically-evoked dopamine release with fast scan cyclic voltammetry, with the focus being the effect of biomaterial encasement and/or time. Rat TE-NSPs encased in 1% agarose (n=5), 3% MeHA (n=4), and 5% MeHA (n=5) were evaluated at ˜36 DIV. These constructs were incubated with 100 μM L-DOPA (Sigma, D9628) for 30 min before transferring to the recording chamber and perfused continuously throughout the analysis. In this case, dopamine release was analyzed using the Kruskal-Wallis test with Dunn's multiple comparisons test. Constructs fabricated with human iPSC-derived dopaminergic neurons (OD: 398 μm; ID: 160 μm) were assessed based on the following groups: 23-24 DIV (3% MeHA: n=3; 5% MeHA: n=3), 29-32 DIV (1% agarose: n=1; 3% MeHA: n=2; 5% MeHA: n=1), and 56-58 DIV (3% MeHA: n=3). In certain cases, L-DOPA was added to determine its effect on dopamine release.

Individual TE-NSPs were transferred to a recording chamber and perfused at 37° C. with media bubbled with 95% O₂ and 5% CO₂ and containing Neurobasal, 2.0 mM L-glutamine, and ascorbic acid (100 and 200 μM for rat and human cells, respectively). A bipolar stimulating electrode (Plastics One) was placed to span the region of interest, and a carbon fiber electrode (150-200 μm outer fiber length, 7 μm diameter) was located in this same area. In all cases the region of interest was the dopaminergic aggregate, except where noted. For stimulation, the bipolar electrode was set to apply a monophasic+ electrical train of 10 pulses of 5 ms width at 20 Hz and an amplitude of 8 V. For recording, the potential of the carbon fiber electrode was scanned linearly from −0.4 V to 1.2 V to −0.4 V vs. Ag/AgCl at a rate of 400 V/s with a voltammeter/amperometer (Chem-Clamp; Dagan Corporation). The stimulation train was applied at 8 s, and the cyclic voltammograms (CVs) were recorded during the entire session every 100 ms using the Demon Voltammetry and Analysis Software. The current at the peak oxidation potential of dopamine in consecutive CVs, as well as all the CVs obtained during the session, were averaged from 4-6 recording runs within the same location 8-12 min apart. This averaging was done to minimize the noise and emphasize the signal. The currents were converted to dopamine concentration with a calibration curve. We recorded the elicited current after injection of 1.5, 3, and 6 μM dopamine hydrochloride (Sigma, H8502) and obtained the slope of the regression of concentration vs. current. The dopamine in this case was dissolved in deionized water with 126 mM NaCl, 2.5 mM KCl, 1.2 mM NaH2PO4-H2O, 2.4 mM CaCl2-2H2O, 1.2 mM MgCl2-6H2O, 25 mM NaHCO₃, and 0.4 mM L-ascorbic acid (all from Sigma).

### Transplantation of TE-NSPs

Male Sprague Dawley rats (8-11 weeks, 275-290 g; Charles River) were used for transplants of MeHA-encased rat TE-NSPs at 14 DIV, with a terminal time point of 2 weeks (3% MeHA: n=3; 5% MeHA: n=3). Rats were anesthetized with 5% isoflurane (Midwest Veterinary Supply, 193.33165.3) and mounted in a stereotactic frame, with anesthesia being maintained at 2.5%. The scalp was disinfected with betadine, and 2.0 mg/kg bupivacaine (Hospira, 0409-1161-19) was injected in the incision area. The incision was made along the midline of the scalp, and a 5 mm craniectomy was made at −5 mm AP and +2.2 mm ML relative to Bregma. TE-NSPs were pulled into a needle (Vita Needle; OD: 534 μm, ID: 420 μm) containing a rod that lay directly in contact with the construct. A protective polyurethane tubing (OD: 686 μm; ID: 559 μm; Microspec Corporation) was cut to fit the needle, sealed on one end, and used to cover the outer area of the needle. The needle was inserted into a Hamilton syringe attached to a stereotactic arm, and its plunger was put directly in contact with the exposed rod coming out of the needle. The stereotactic arm was adjusted to be at 38° relative to the horizontal, the dura was opened, and the needle was lowered ˜12 mm into the brain. Then, the sheath was pulled up with forceps to break the seal, and the TE-NSPs were laid out by pulling up the needle ˜5 mm while a stationary arm maintained the plunger in place. After 2 min, the needle was pulled out entirely, the scalp was sutured, and the animals received a subcutaneous injection of 2.0 mg/kg meloxicam (Midwest Veterinary Supply, 577.30200.3). The animals were sacrificed with transcardial perfusions with heparinized saline followed by 10% formalin. The brains were extracted, post-fixed overnight in 10% formalin, and stored in 1×PBS at 4° C.

### Immunohistochemistry

Thick brain sections were subjected to a clearing and immunohistochemistry protocol to visualize the entire constructs and assess their cytoarchitecture and survival. A vibratome (Leica, VT1000S) was utilized to obtain 2 mm thick sagittal sections containing the implanted constructs. The sections were dehydrated with increasing concentrations of methanol (Fisher, A412) and then incubated overnight at room temperature with 66% dichloromethane (Sigma, 270997) and 33% methanol for delipidation and refractive index matching. Afterwards, the sections were bleached overnight in chilled 5% H₂O₂ (Sigma, H1009), rehydrated with decreasing concentrations of methanol, permeabilized for 2 days at 37° C., and blocked for 2 days at 37° C. Permeabilization was performed using 0.16% Triton X-100, 20% dimethyl sulfoxide (DMSO; Sigma, 276855), and 0.3 M glycine (Bio-Rad, 161-0718) in 1×PBS, while the blocking solution consisted of 0.16% Triton X-100, 6% horse serum, and 10% DMSO in 1×PBS. Afterwards, the sections were exposed to primary and secondary antibodies for 7 days at 37° C. each, with washing steps in between. The primary antibody buffer was 3% horse serum and 5% DMSO dissolved in 1×PBS with 0.2% Tween-20 (Sigma, P2287) and 10 μg/mL heparin (Sagent Pharmaceuticals, 25021-400-10), while the secondary buffer contained 3% horse serum and the same solvent. We used primary antibodies for TH ( 1/100, sheep, Abcam, ab113) and GFP ( 1/100, mouse, Aves, GFP-1020) and the appropriate Alexa-Fluor secondaries ( 1/250). The sections were then dehydrated with increasing concentrations of methanol, exposed to Visikol® HISTO-1™ overnight at room temperature, and incubated with Visikol® HISTO-2™ at least for 2 hr before imaging.

### Results:

### Characterization of the Mechanical Properties of MeHA and Agarose Hydrogels

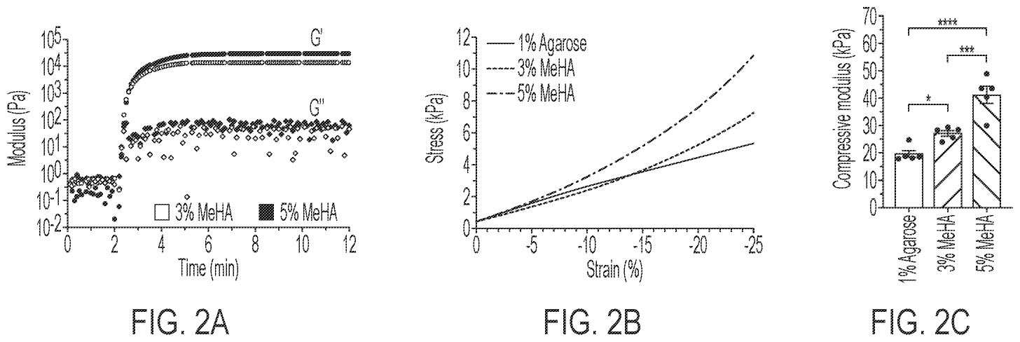

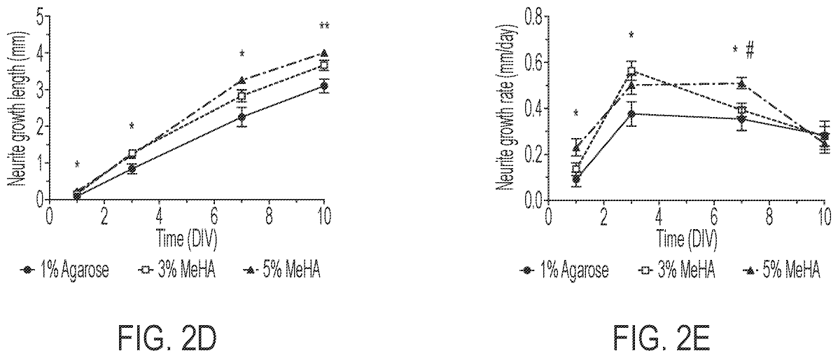

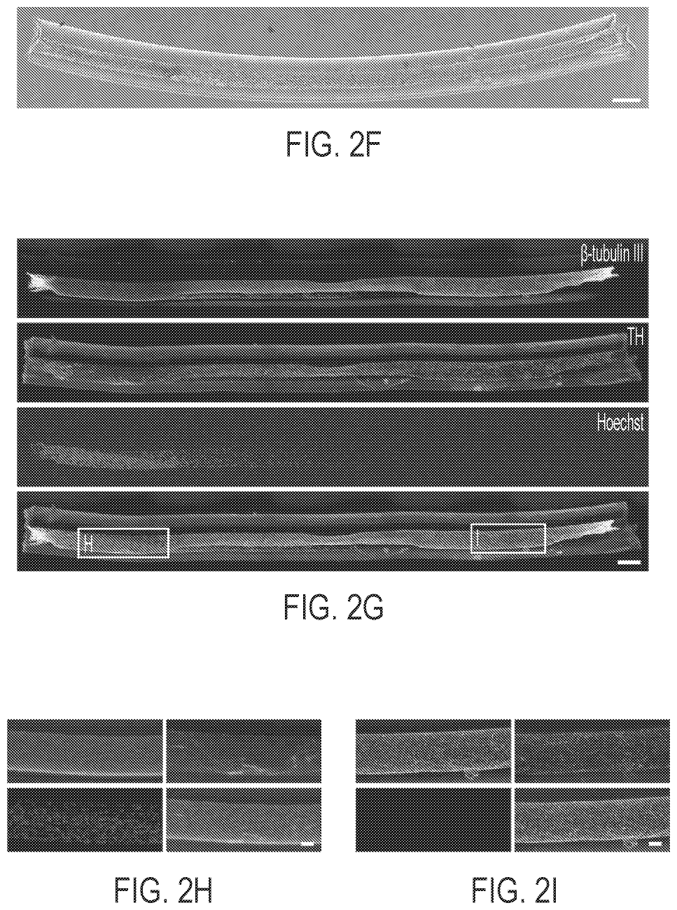

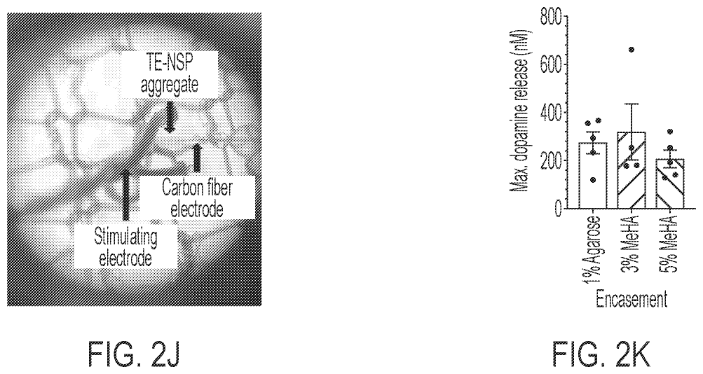

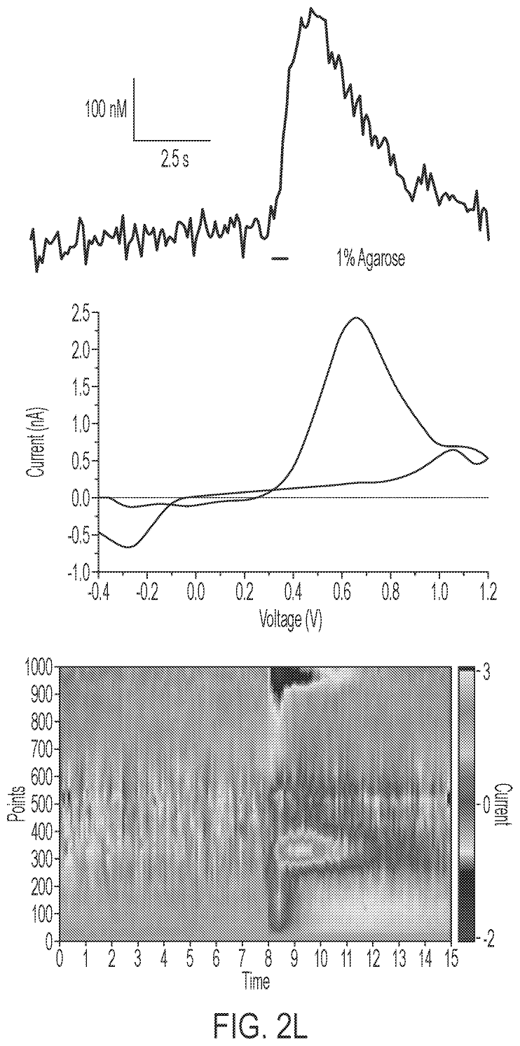

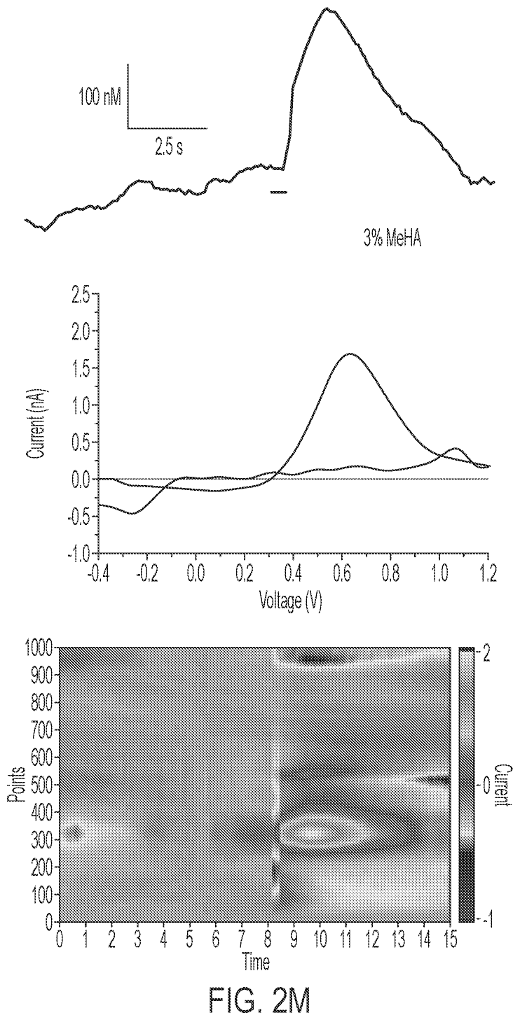

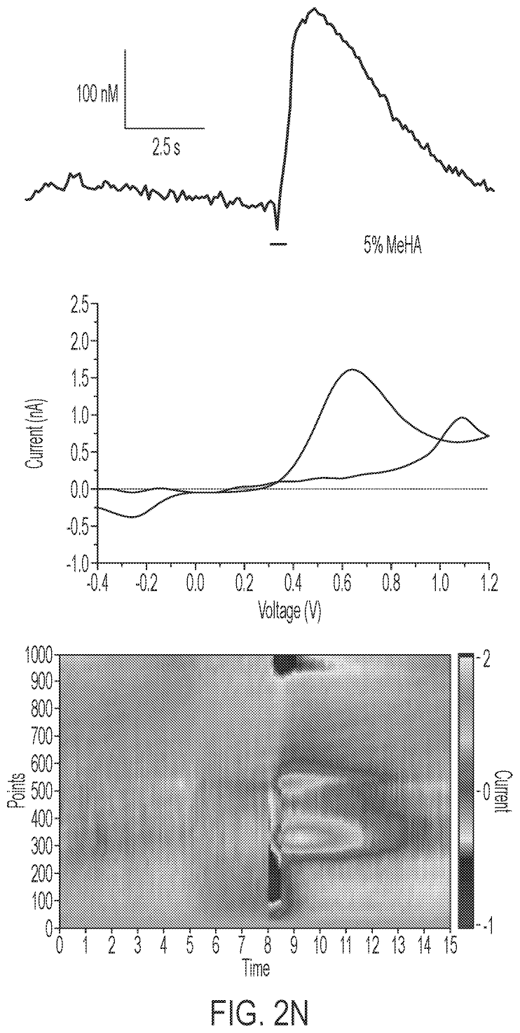

We have traditionally fabricated TE-NSPs encased in 1% agarose hydrogels (1), but for the next generation of these constructs we chose HA hydrogels because of their bioactivity and the modifications available to expand the capabilities and functionality of TE-NSPs. We modified ˜40% of the hydroxyl groups in the N-acetyl-D-glucosamine units of HA to obtain MeHA following existing protocols. MeHA hydrogels can be created by radical photopolymerization reactions where light and an initiator molecule generate free radicals that react with the methacrylate reactive group. Indeed, hydrogels were formed by irradiating solutions with 10 mW/cm² UV light, as confirmed with rheological measurements showing an increase in the storage (G′) modulus as a function of time after exposure to light to values much greater than the loss (G″) modulus (FIG. 2A). As expected, the value for G′ for a 5% MeHA hydrogel was greater than that for 3% MeHA. Moreover, the profiles demonstrated that a light exposure time of ˜5 min was sufficient to reach a plateau in mechanical properties (based on the plot without a logarithmic scale). After confirming that MeHA hydrogels could be photocrosslinked, we studied how their mechanical properties compared with our traditional agarose hydrogels by obtaining the compressive modulus from stress vs. strain curves (FIG. 2B). The compressive modulus for a 5% MeHA hydrogel was significantly greater than the value for 3% MeHA; importantly, both MeHA hydrogels were significantly stiffer than 1% agarose (FIG. 2C).

### MeHA is a Suitable Encasement for TE-NSPs Based on Growth and Functional Outcomes

We evaluated the feasibility of using MeHA to encase TE-NSPs by fabricating these constructs with rat embryonic dopaminergic neurons, our conventional cell source, and testing outcomes of growth, cytoarchitecture, and functionality in vitro. Initially, we confirmed that MeHA hydrogels could be produced in the shape of a hollow micro-column that could be filled with polymerized collagen and laminin. Qualitatively, we have consistently observed a high yield of MeHA micro-columns with ECM filling the lumen axially and longitudinally. We were then able to produce MeHA-encased TE-NSPs with a rat dopaminergic aggregate in ˜0.5 cm micro-columns. We characterized the role of biomaterial type and time on TE-NSP neurite growth, with the former having a significant effect on length (P=0.0002) and rate (P=0.0011) and the latter on both outcomes (P<0.0001). All mean neurite lengths were higher for MeHA groups relative to agarose, with significant differences being observed between 1% agarose and one of the MeHA groups at all time points (FIG. 2D). The axons extended completely through the lumen by 10 DIV. Overall, the greatest growth rates were observed in the 3-7 DIV range for all types of encasements, and the lowest were seen at 1 and 10 DIV (FIG. 2E). The mean growth rates were generally higher for MeHA in all cases, and there were significant differences between 1% agarose and one of the MeHA groups at 1, 3, and 7 DIV. In one case, the growth rate in 5% MeHA was significantly higher than 3% MeHA at 7 DIV. MeHA constructs exhibited the correct cytoarchitecture and phenotypic expression; we observed segregated neuronal somata and dopaminergic axon tracts, similar to the nigrostriatal pathway, as noted from Hoechst⁺ nuclei and TH⁺ neurites, respectively (FIGS. 2F-2H). While we observed instances of neuronal migration/dispersal along the axon tracts, the presence of neurons in the axon region was minimal.

Following these structural characterization studies, we examined the functionality of TE-NSPs encased in MeHA in terms of electrically-evoked dopamine release. We used fast-scan cyclic voltammetry (FSCV), a technique with high temporal/spatial resolution used to measure changes in neurotransmitter concentration based on the currents elicited by the oxidation or reduction of the released molecules. For this purpose, we introduced a carbon fiber electrode into the aggregates in TE-NSPs to record extracellular dopamine release after electrical stimulation (FIG. 2J). The peak evoked dopamine concentration was calculated from one location within the dopaminergic aggregate after averaging several recording sessions. Rat TE-NSPs in 3% and 5% MeHA had an average dopamine release of 317.6±115.7 nM and 206.289±35.69 nM, respectively, in comparison to 273.1±45.23 nM when encased in 1% agarose (FIG. 2K). These evoked concentrations were not statistically different (P=0.6175) and, as such, we did not observe a significant effect of the biomaterial on functionality. In all three encasement types, the cells responded to stimulation (FIG. 2L-2N, top), and the released molecules exhibited the chemical signature of dopamine. In particular, the cyclic voltammograms (CVs) at peak release (FIG. 2L-2N, center) showed clear current peaks at the oxidation potential and troughs at the reduction potential of dopamine around 0.6 V and −0.3 V, respectively. CVs recorded as a function of time at a high scan rate also show that characteristic dopamine signal throughout the duration of release (FIG. 2L-2N, bottom).

### MeHA-Encased, Rat TE-NSPs can be Implanted Along the Nigrostriatal Pathway in Rats

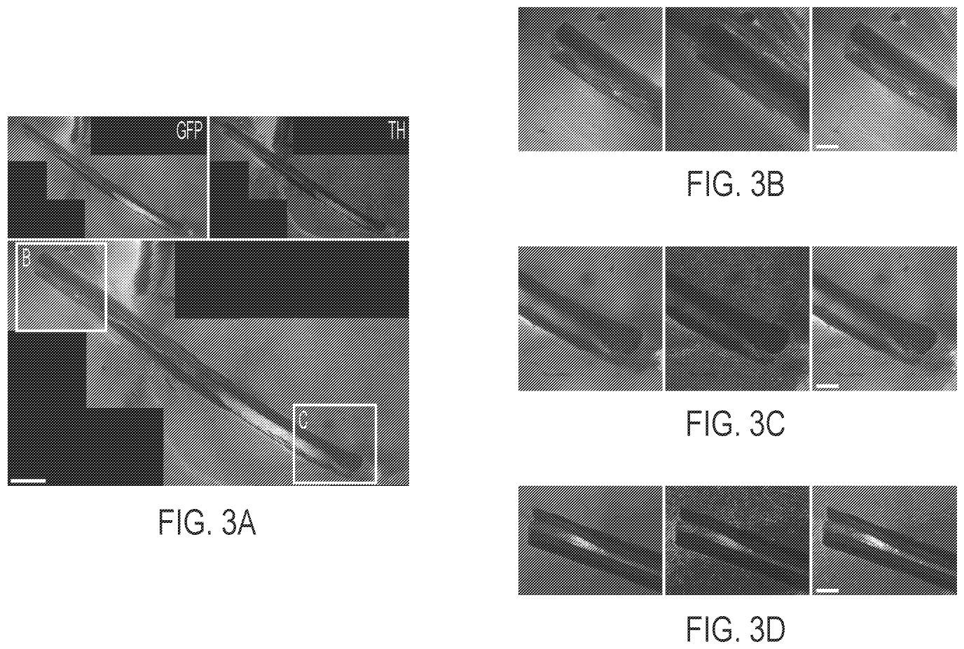

A crucial consideration for the feasibility of using MeHA to encase TE-NSPs was whether these constructs could be implanted in rats, maintain their structure during and after delivery, and survive. Thus, after 14 DIV MeHA-encased rat TE-NSPs were stereotactically implanted to span the region between the SNpc and the striatum in rats (FIG. 3A). The MeHA micro-columns could be loaded into needles and manipulated without observable damage to the integrity of the hydrogel or the cells and axon tracts. Through tissue clearing and immunohistochemistry we observed that the constructs could be delivered to span the nigrostriatal pathway, with one end in the striatum (FIG. 3B) and the other end proximal to the nigral cell bodies (FIG. 3C), and maintain their cytoarchitecture and integrity for at least 2 weeks. We also observed instances of outgrowth from the TE-NSP into host tissue (FIG. 3C). Moreover, host TH+ projections were sometimes seen growing alongside the micro-column, and an environment permissible to axon growth indicates that the hydrogel likely does not present adverse effects to the host brain environment (FIG. 3D). Furthermore, we did not observe signs of mass effect, inflammation, bleeding, or astrocyte reactivity in the vicinity of the implants.

Human iPSC-Derived Dopaminergic Neurons can be Employed to Fabricate TE-NSPs with Various Dimensions

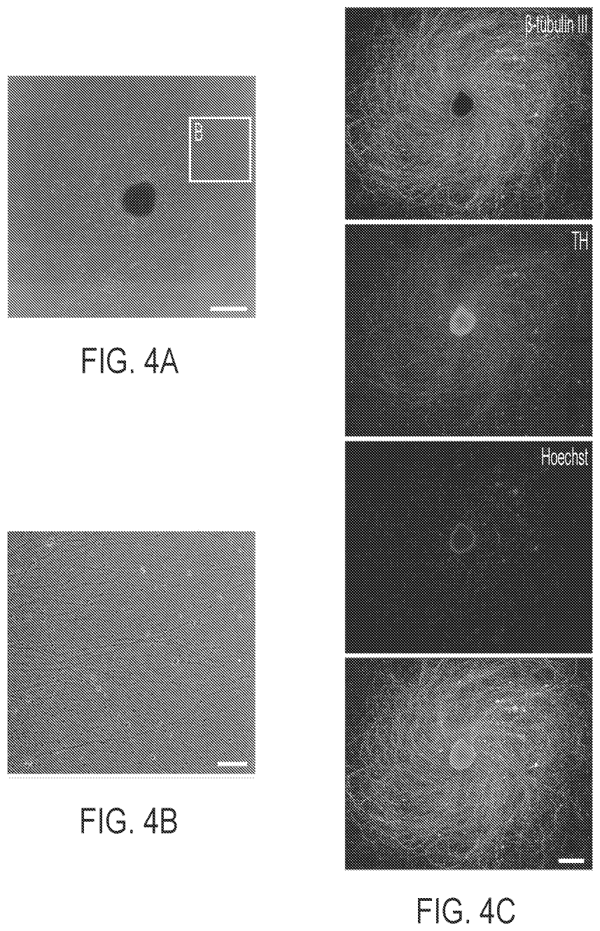

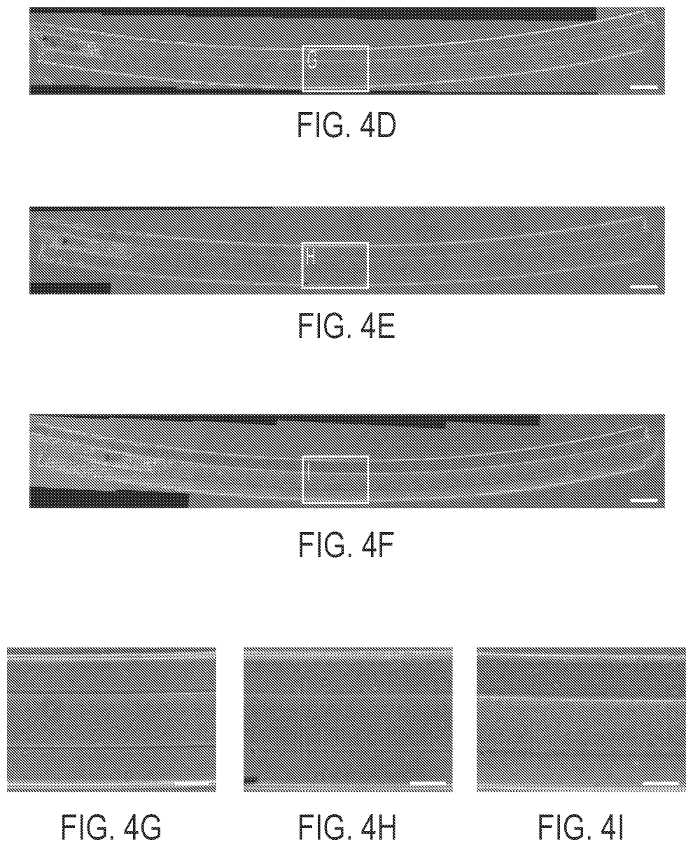

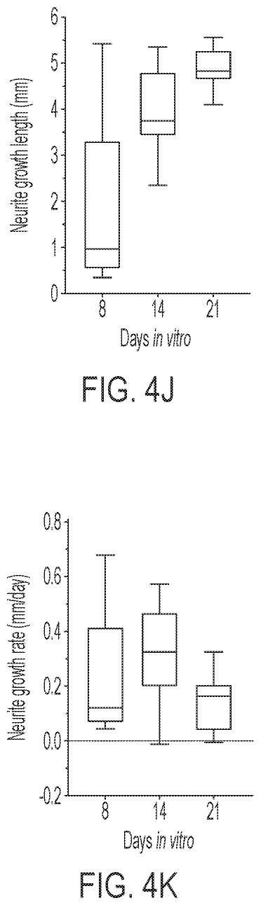

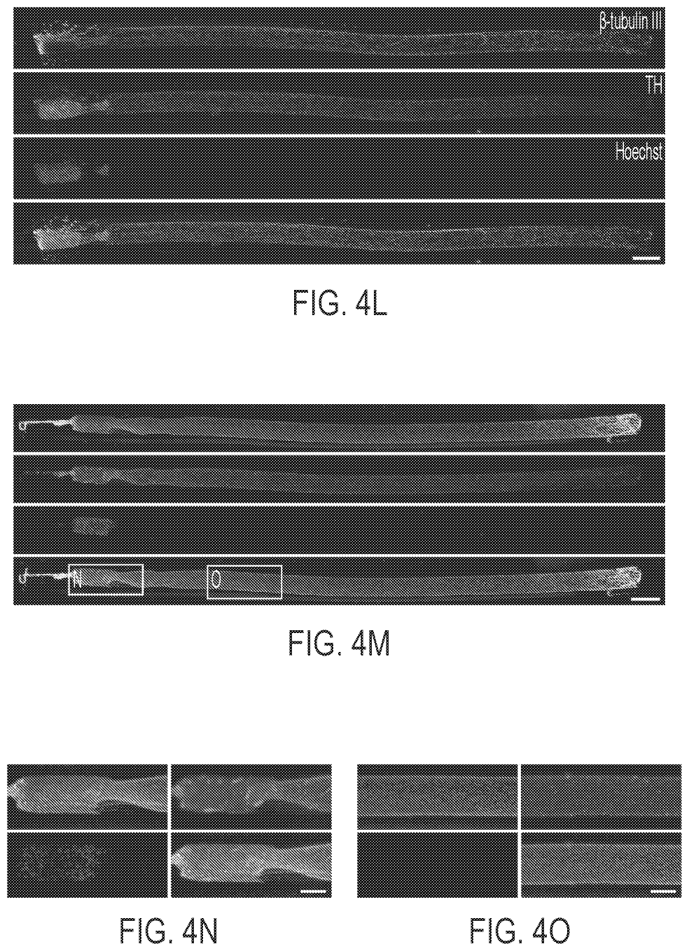

Ultimately our goal was to transition to more clinically relevant TE-NSPs fabricated from human dopaminergic neurons. We obtained these cells after differentiating human iPSCs in 2D and aggregating them by centrifugation in custom-made micro-wells at ˜40 dd, after which they were plated in 2D or within micro-columns. This time point was chosen balancing the need for dopaminergic fate specification, sufficient TH expression (>50%) when plating, proper maturation at the time of implantation, and timeliness in TE-NSP fabrication. We first needed to confirm that human dopaminergic neurons could be cultured as aggregates in laminin-coated surfaces. These aggregates exhibited extensive neurite growth in all directions from the entire circumference (FIGS. 4A, 4B). Immunolabeling was then used to corroborate the TH phenotype of these aggregates. The images showed dense dopaminergic axonal projections around the aggregate and the presence of migrating and/or dispersed Hoechst+ neurons from the aggregate throughout the surrounding area at 24 DIV (FIG. 4C).

Having confirmed that the neurons survive and grow after aggregation, we proceeded to create TE-NSPs with 3% MeHA and these human aggregated neurons. We only made human constructs with 3% MeHA because 3 and 5% MeHA yielded generally similar growth and functional results with rat neurons, as previously discussed, and we wanted to maintain the encasement type consistent when testing human neurons. In addition, 3% MeHA micro-columns could be easily handled manually even though they were noticeably softer than 5% MeHA. We started making TE-NSPs with dimensions relevant for implantation to span the endogenous nigrostriatal pathway in rats, having an OD and ID of 398 and 160 μm, respectively, and a length of ˜0.5 cm. Representative phase contrast images of these human TE-NSPs showed neurite growth throughout the entire length of the micro-column (FIG. 4D-4F), with the density increasing over time from 8 to 21 DIV (FIG. 4G-41). The neurite growth length and rate in these constructs was measured over time from three independent batches. Average growth lengths were 1.96±0.43, 3.88±0.22, and 4.89±0.10 mm at 8, 14, 21 DIV, respectively, while the median values were 0.97, 3.74, and 4.82 mm. These results and the boxplots suggested that the growth at initial time points had a wide distribution, although TE-NSPs consistently grew through most or the entire length of the micro-columns by 14 or 21 DIV (FIG. 4J). At 8, 14, and 21 DIV the mean growth rates were 0.24±0.05, 0.32±0.04, and 0.14±0.03 mm/day and the median rates were 0.12, 0.32, and 0.16 mm/day, respectively. The growth rate distribution at 21 DIV was not as wide as those at 8 and 14 DIV due to the neurites having less space to grow after arriving at the end of the columns (FIG. 4K). We noted the correct cytoarchitecture of dopaminergic TH+ axonal tracts projected from a cluster of neurons restricted to one area in constructs fixed at 23 DIV (FIG. 4L), a time relevant for implantation given that TE-NSPs typically achieve full axon growth by 21 DIV. This structure, resembling the cytoarchitecture of the nigrostriatal pathway, was maintained even after 2 months in vitro (FIG. 4M-40). This suggests that MeHA-encased human TE-NSPs can sustain the integrity and phenotype of their axonal tracts long-term at least in vitro, a requisite for chronic implantation. Moreover, in contrast with 2D aggregate cultures, we observed no notable dispersal or migration from the aggregates within the micro-columns.

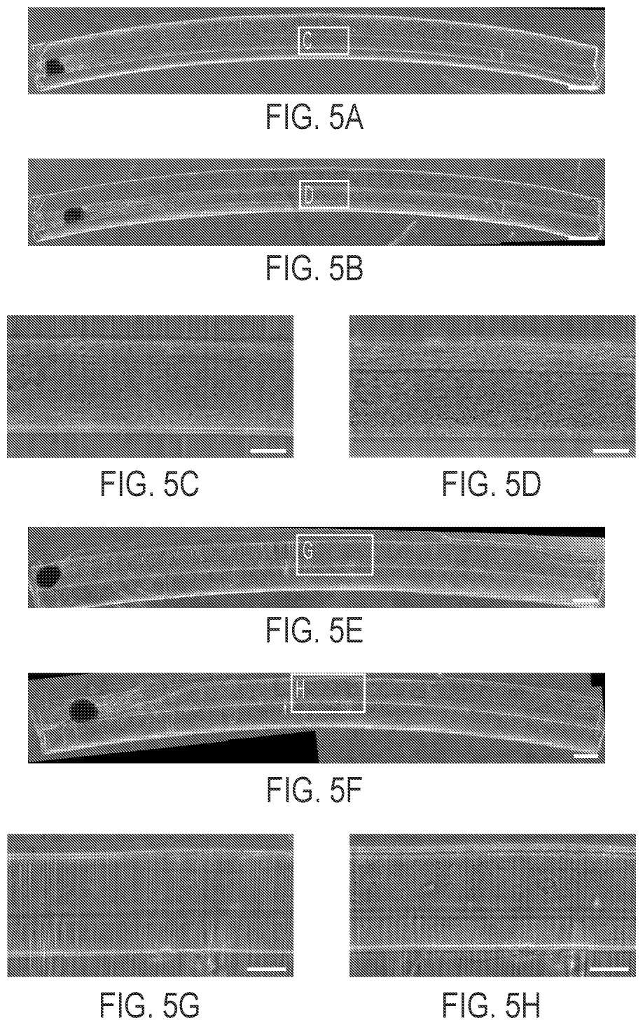

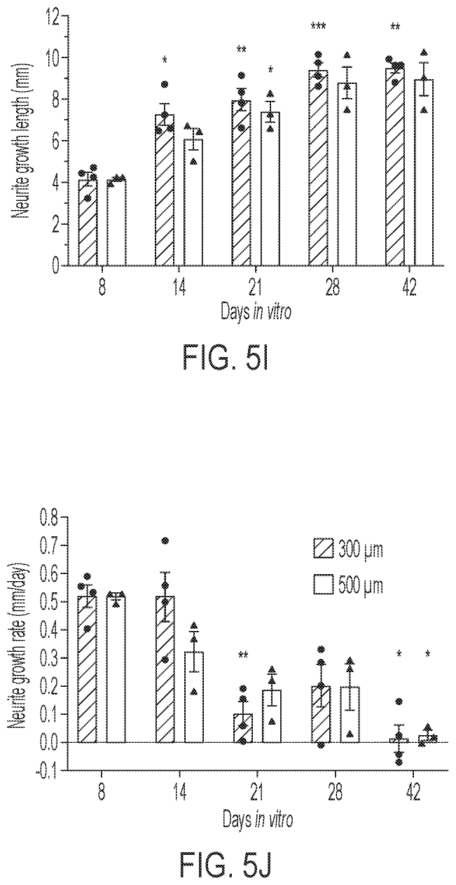

We also fabricated human TE-NSPs with greater diameters and lengths to investigate if these constructs could be scaled up to accommodate the cell number and structural requirements for implantation in larger animal models and humans. MeHA-encased human TE-NSPs could be made to have axon tracts spanning the lumen of micro-columns with an ID of 300 μm (FIGS. 5A, 5B) and 500 μm (FIGS. 5E, 5F) and lengths at least up to 1 cm. In both cases, the axon density qualitatively increased over time (FIGS. 5C, 5D, 5G, 5H) and seemed to be greater in a 300 μm lumen because of the lesser space for growth (FIGS. 5D, 5H). It was concluded that diameter had no significant effect on growth lengths (P=0.2475) or rates (P=0.3424), and there were no statistical differences between 300 and 500 μm groups at each time point (FIGS. 5I, 5J). On the other hand, time had a significant effect on neurite growth length (P<0.0001) and rates (P=0.0010) as expected. The maximum growth rates were observed at initial time points in the 8-14 DIV range around 0.52 mm/day for both groups, while lowering to 0.10-0.20 mm/day at 21-28 DIV.

### MeHA-Encased, Human TE-NSPs Release Dopamine Upon Electrical Stimulation

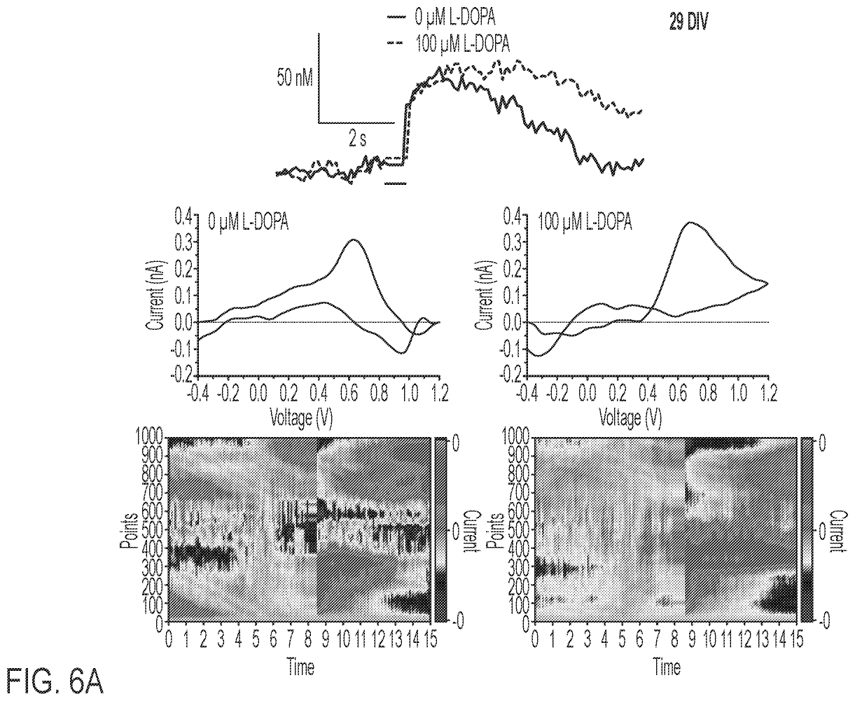

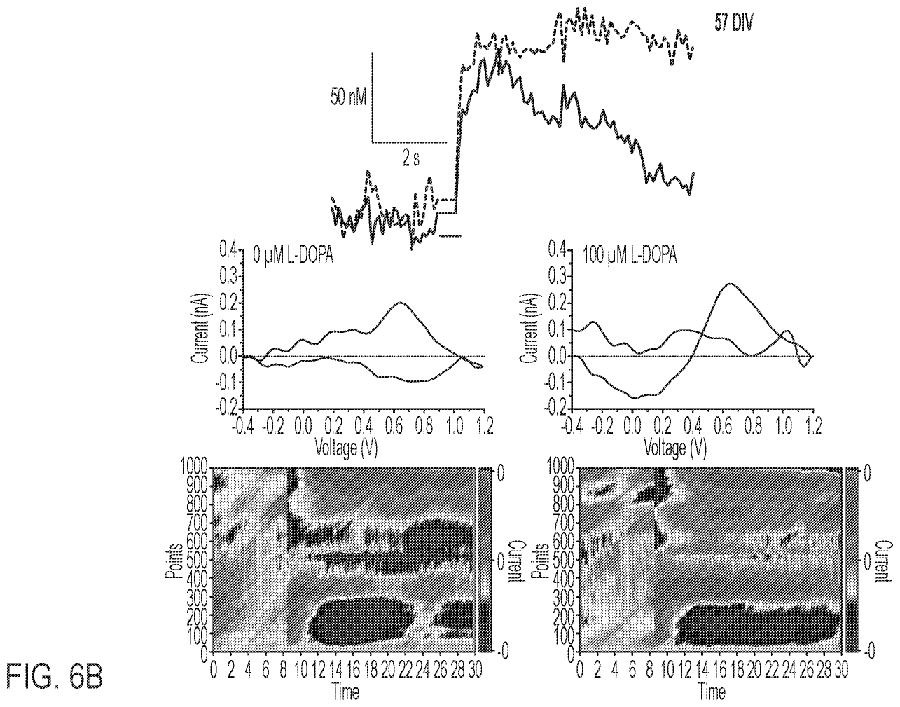

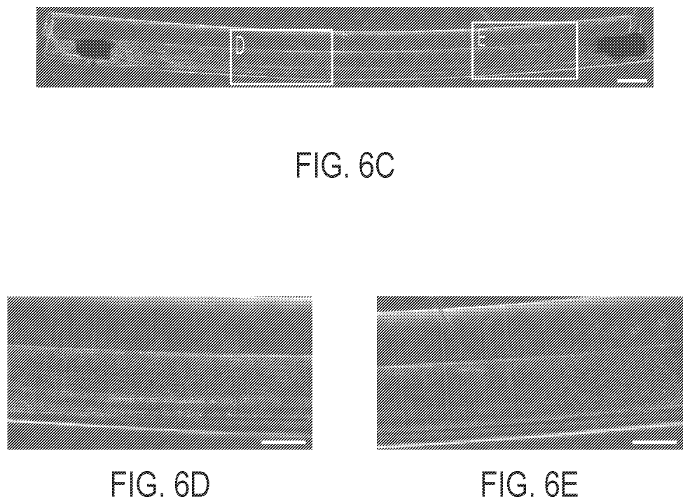

We employed FSCV to detect the release of dopamine after electrical stimulation in the aggregate of human TE-NSPs and to corroborate the functionality of these constructs. After placing both the stimulating and carbon fiber electrodes in the aggregate, we observed examples of evoked release of 56 nM (FIG. 6A) and 107 nM (FIG. 6B) after approximately 30 and 60 DIV, respectively. The CVs verified the dopaminergic identity of the released molecules as seen by the oxidation peak at ˜0.6 V and reduction trough close to −0.3 V (FIGS. 6C, 6D). These human constructs could also respond to perfusion with L-DOPA; dopamine release in the same area was sustained for longer, as observed in the concentration traces (FIGS. 6A, 6B) and the extended appearance over time of the oxidation peak for dopamine in the CV colorplots (FIGS. 6C, 6D, bottom). Despite these results, we did not always obtain evoked dopamine release from all tested TE-NSPs, particularly when comparing between different batches of differentiation (data not shown). A final show of the parallels between TE-NSPs and the native nigrostriatal pathway would be the co-culture of the human dopaminergic axons with striatal neurons given their integration in the brain. We were able to grow human TE-NSPs with a rat embryonic striatal neuron aggregate placed in the micro-column at the end opposite the human dopaminergic aggregate. While both cell types extended neurites, the human dopaminergic aggregate projected long axons that were detected to be in close proximity to striatal projections in phase contrast images (FIGS. 6E-6G).

Standard treatments for PD (e.g., L-DOPA) therapy generally involve discontinuous drug delivery, which can cause severe motor fluctuations with chronic exposure. DBS electrodes provide inherently non-specific stimulation and are implanted downstream of the striatum, thus circumventing the cause of PD. Moreover, these stiff inorganic electrodes are invasive and typically produce a chronic foreign body response diminishing their effectiveness. Cell replacement approaches have traditionally involved the transplantation of fetal- or stem cell-derived dopaminergic neurons only in the striatum. In contrast, TE-NSPs represent a biologically-inspired strategy to reconstruct the dopaminergic fiber tracts in the nigrostriatal pathway, thereby targeting both the loss of dopaminergic neurons/axons and striatal innervation and dopamine input at the core of the pathophysiology of PD. TE-NSPs would ideally improve upon the discontinuous effects of L-DOPA and the lack of specificity of DBS by delivering dopamine in a natural, synaptic-based, and spatially-specific manner. Apart from this, our human TE-NSPs can possibly advance conventional cell replacement approaches by employing a clinically relevant cell source that is more available than fetal grafts and that may allow the development of personalized implantable dopaminergic axons. These constructs also featured an HA hydrogel encapsulation, which serves to protect cells and axons during and after implant in comparison with current stem cell-derived transplants delivered as cell suspensions. This may allow us to tune degradation profiles and to provide other functionalities like the local presentation or controlled release of pro-survival molecules to improve in vivo outcomes.

Our previous iterations of TE-NSPs have been fabricated with agarose hydrogels and rat embryonic ventral midbrain neurons. Here, we transitioned to using HA (in its modified form MeHA) as the biomaterial for the encasement of the dopaminergic axon tracts. HA is an unsulfated glycosaminoglycan that is the major backbone of the brain ECM and can bind to neural cells through cell surface receptors such as CD44 and CD168. It forms complexes with lecticans, a type of chondroitin sulfate proteoglycan that includes aggrecan, versican, neurocan, and brevican. These complexes offer the structural support and modulation of development, cell signaling, intercellular interactions, and synaptic plasticity that are key to brain function and participate in the response to injury. Degradation of HA in injury has been associated with angiogenesis and neuroimmune and inflammatory responses according to the molecular weight of the fragments. HA hydrogels have been applied for 3D stem cell encapsulation and control of their expansion, migration, and differentiation. In terms of neural applications, HA hydrogels have been utilized to differentiate human iPSC-sourced neural progenitor cells (NPCs) and augment survival and behavioral outcomes in models of stroke. In a study related to ours, human ESC-derived dopaminergic neurons were encapsulated in HA to mature the cells in vitro and protect them during transplantation. Despite this, there are no published data characterizing human iPSC-sourced dopaminergic neurons in HA. This biomaterial can be used to form hydrogels ideal for our application when the backbone is modified with certain functional groups. We used MeHA given its status as the most common form of modified HA for photocrosslinking. Photopolymerization paradigms permit having spatial control of gelation and properties and in situ cell encapsulation. MeHA and other modified HA hydrogels have also been used extensively for 3D-bioprinting, which should allow for standardized manufacturing of micro-column as described herein. Previous manufacturing methods often produced micro-columns inherently have off-center lumens with variable wall thicknesses along the constructs and between batches. This could increase the likelihood of ruptured micro-columns and exposure of cells and axons to differential stiffnesses. Thus, using HA and 3D-bioprinting to expected to allow the manufacture of TE-NSPs with less batch-to-batch variability and a greater potential in the clinic.

Here, we were able to show that MeHA can be applied to build hollow micro-columns containing a lumen with polymerized collagen and laminin. Neuronal aggregates sourced from rat embryonic ventral midbrains were able to extend long axon tracts that stained for TH, a key dopaminergic marker. Growth and functional outcomes were compared to 1% agarose micro-columns given that this biomaterial has been our historical standard when fabricating TE-NSPs and other micro-tissue constructs. Our results suggested that the type of hydrogel had a significant effect on growth length and rate, with MeHA-encased constructs exhibiting significantly greater outcomes relative to 1% agarose. At 1 DIV, which may imply that the aggregates may require time to properly adhere and adapt to their environment prior to extending neurites at higher growth rates. Growth rates were lowest We confirmed that MeHA-encased TE-NSPs retained their functionality by releasing dopamine upon electrical stimulation in levels comparable to time-matched constructs encased in 1% agarose. Overall, we validated that MeHA can be used to make TE-NSPs that structurally and functionally resemble the nigrostriatal pathway.

The effects of the hydrogel on growth could be attributed to MeHA itself providing a microenvironment more conducive for neurite growth than previous materials, for example, agarose. For example, HA could be more amenable to cells given its crucial role in vivo in the brain ECM. It may be better at retention and local presentation of growth factors present in the media to cells in comparison to agarose. MeHA could also promote a more consistent superior polymerization of the collagen and laminin core, thus providing a 3D space more agreeable for neurite growth. On the other hand, the improved growth in MeHA could be a result of the distinct mechanical properties of MeHA and agarose, as exemplified by the greater compressive moduli for the former. We have previously shown that axons have the highest density of growth along the hydrogel-ECM interface in the circumference of the lumen. As such, while the neurites use the ECM core for growth throughout the width of the lumen, they also interact with and use the hydrogel as a substrate for growth, and the mechanical properties of the biomaterial may thus directly impact cell behavior. Indeed, it has been extensively studied that neural cells and other types sense and respond to substrate stiffness. Specifically, it has been widely reported that there is an inverse relationship between neurite growth and substrate stiffness in studies using mouse hippocampal NPCs seeded on a 3D hydrogel of HA-pentenoate and a poly(ethylene glycol)-bis(thiol) crosslinker, mouse ventral midbrain NPCs in MeHA hydrogels, and chick dorsal root ganglia in 3D agarose hydrogels. In addition, human iPSC-derived NPCs, as single cells or spheroids, exhibited more spreading and attachment in acrylated HA hydrogels and greater neurite outgrowth/density and neuronal differentiation in 3D MeHA hydrogels that were soft relative to stiffer substrates. While our results coincide with stiffness influencing neurite growth, we observed an opposite relationship: the stiffer MeHA micro-columns benefitted growth length and rate when compared to the less stiff 1% agarose. This may imply that other mechanical/physical properties are the driving force in provided constructs, that indeed MeHA itself provides a better environment than agarose separate from stiffness, and/or that the stiffer MeHA provides a more structurally sound encasement that maintains the integrity of the ECM core. On the latter point, it is indeed common to see shrinking or detachment of ECM inside agarose hydrogels, which may disrupt the neurite growth process. Our results may also reflect that we are using a different type of scaffold, one in which the hydrogel substrate is found in the periphery of a 3D micro-column, while previous studies have relied on 2D surfaces or 3D growth in whole blocks/pieces of hydrogel. Indeed, we previously demonstrated improved neurite health in engineered micro-tissues similar to TE-NSPs when grown in 3-4% agarose micro-columns relative to 1-2% agarose. Thus, this direct relationship between stiffness and growth outcomes may be a feature of our micro-column scaffolds.

The present disclosure also confirmed that MeHA micro-columns could be successfully delivered into rat brains along the nigrostriatal pathway and that the cells could survive and retain their axonal cytoarchitecture for at least 2 weeks in vivo. As such, we deemed that MeHA was a feasible encasement for TE-NSPs in terms of structure, function, and transplantation. MeHA is mainly susceptible to enzymatic degradation by hyaluronidases. However, our results indicate that hyaluronidase presence around the implant was not sufficient for noticeable degradation to occur at observed time points. In some embodiments, it may be advisable to have a controllable degradation profile in which there is little to no degradation during the in vitro culture and early implant periods, with degradation becoming more prominent in vivo as time goes on. This would allow for TE-NSPs to grow in 3D with the proper cytoarchitecture and to have sufficient integrity to be properly implanted and to integrate with the host circuitry acutely. Subsequent degradation may enable better and more seamless integration of the dopaminergic axon tracts with the surrounding tissue. Moreover, the rate of degradation is inversely related to stiffness and has other implications. For example, it has been established that implants should have mechanical properties matching the surrounding tissue for better outcomes such as the inflammatory response. Indeed, softer acrylated HA hydrogels with a stiffness matching the mouse brain caused significantly less reactive astrocyte and microglia presence in naive and stroke mouse models, respectively, relative to stiffer hydrogels. Still, the stiffness must be sufficient to ensure the hydrogels hold the shape of a hollow micro-column without disrupting the interior before, during, and after implantation. Key properties, such as degradation and stiffness, can be tuned by optimizing the MeHA macromer concentration, molecular weight of HA, and the degree of methacrylation. Greater control of degradation may be granted by using hydrogels that are more susceptible to hydrolysis or cell-based degradation. For example, HA hydrogels sensitive to hydrolysis have been created by modifying HA to have methacrylates bound to lactic acid or caprolactone, which are responsive to ester hydrolysis. Di-thiol peptides sensitive to cleavage by matrix metalloproteinases expressed by cells have also been used as part of the sequential crosslinking of acrylated HA hydrogels.

Other types of HA could also be used to make TE-NSPs depending on the desired properties (e.g., degradation, stiffness, toughness, binding of cell-responsive peptides, retention and release of growth factors). Previous studies have characterized double-network hydrogels consisting of a primary network resulting from the non-covalent guest-host assembly of HA modified with β-cyclodextrin and HA with adamantane and a secondary one with thiol-based crosslinking of MeHA. The two networks could also be covalently tethered when the polymers in the primary network were also methacrylated. The guest-host assembly conferred self-healing properties by dissipating and transferring stress to protect the covalent bonds. On the other hand, changing the thiol-to-methacrylate ratio tuned the compressive elastic moduli of the hydrogels. These hydrogels could allow TE-NSPs to recover from mechanical deformation without failure and provide greater resilience to handling during culture and implantation. Notably, this could be a possible avenue for shaping TE-NSPs during implantation to fit the more complex curved geometry of the human nigrostriatal pathway. For example, with norbornene-modified HA, the norbornenes can have crosslinks only with the addition of di-thiol molecules. By modulating the ratio of thiols to norbornenes, it can be ensured that there will be unreacted norbornenes remaining for subsequent reactions. This paradigm could be applied for TE-NSPs encased in micro-columns that are first crosslinked and then bound with thiol-containing peptides or proteins in a secondary reaction. These molecules could influence survival, adhesion, neurite growth, among other relevant in vitro and in vivo outcomes. In addition, TE-NSPs could be further modified to retain or deliver growth factor and drugs, as HA hydrogels have been widely used for this purpose based on diffusion, protease-sensitive, or hydrolytic degradation, on-demand force-mediated and light-triggered processes, and affinity to chemical groups. We foresee that HA will significantly improve TE-NSPs and other engineered micro-tissues by expanding their range of applications, design control, and prospects for successful clinical outcomes.

Having validated the use of an HA-based hydrogel to encase TE-NSPs, we then created these constructs with human iPSC-derived dopaminergic neurons. These neurons allow for fabricating patient-derived, implantable dopaminergic axon tracts, circumventing the ethical and practical limitations of human fetal- or ESC-sourced neurons. These neurons were differentiated in 2D for ˜40 days following established protocols and then aggregated for seeding within the micro-columns. The present disclosure describes the feasibility of combining an HA-based hydrogel encasement and human iPSC-derived dopaminergic neurons to fabricate TE-NSPs. We found that the human dopaminergic aggregates could grow axon tracts to ˜1 cm by 28 DIV within micro-columns. To our knowledge, this is the first report of human iPSC-derived dopaminergic engineered tissue exhibiting unidirectional axon growth reaching lengths up to ˜1 cm in vitro. Human TE-NSPs exhibited the desired cytoarchitecture of neuronal cell bodies restricted to the end of the micro-columns with aligned axon tracts longitudinally spanning the lumen and expressing a dopaminergic phenotype at least up to 2 months in vitro. The human TE-NSPs released dopamine to the extracellular space within the aggregates upon electrical stimulation, with concentrations increasing with time. Notably, we observed dopamine release in the 50-110 nM range between 30-60 DIV using FSCV, which is the range that is estimated to cause a functional effect in parkinsonian rats. Specifically, constructs cultured for longer were able to release more dopamine after stimulation, suggesting ongoing maturation and concomitant increase in dopamine release machinery. This assessment of somatodendritic dopamine release served as verification for the health, phenotype, and functionality of TE-NSPs. Somatodendritic release in the SNpc is also an important aspect of the native nigrostriatal pathway, where it is involved in the modulation of neuronal activity in the SNpc and SNpr and distal dopamine release in the striatum. It was not surprising that we did not detect evoked dopamine in the axonal tract region given that TE-NSP axons were in their developing, growth phase and did not have an end-target for integration. Studies have highlighted the absence of or low capacity for evoked neurotransmitter release in developing mammalian neurons of the central nervous system. Another noteworthy observation during FSCV analysis was that the CV colorplots for human TE-NSPs appeared to be more noisy or show signal peaks other than those characteristic for dopamine when compared to the much cleaner plots for rat TE-NSPs. This may highlight differences in fate commitment, functional maturation, or cell content between the rat embryonic and the human iPSC-derived dopaminergic neurons.

We utilized an existing protocol to differentiate human ESCs and iPSC into dopaminergic neurons based on the coexpression of the floor-plate and roof-plate markers FOXA2 and LMX1A, respectively. These neurons had more than 50% expression of TH and exhibited dopamine release, membrane potential oscillations at frequencies characteristic of SNpc neurons, and markers such as G-protein-regulated inward-rectifier potassium channel 2 (GIRK2) and calbindin, which are characteristic for A9 and A10 dopaminergic neurons, respectively.

Another aspect of construct manufacturing is consideration around the ratio of A9 to A10 dopaminergic neurons after differentiation and present within the TE-NSPs, given the role of these cell types in functional dopamine restoration. The A9 dopaminergic neurons extend axons mainly to the dorsolateral striatum and are selectively lost in PD, while the A10 cells of the ventral tegmental area project to the ventral striatum and limbic regions and are not heavily impacted. Certainly, the intrinsic capacity of cells to recognize proper outgrowth targets has been highlighted by the inability of non-A9 dopaminergic neurons (e.g., dopaminergic neurons of the olfactory bulb, ventral forebrain neurons) to innervate the dorsolateral striatum. Consequently, studies in 6-OHDA rats have shown that mouse-sourced intrastriatal grafts enriched in A9 neurons (˜65% GIRK2+/calbindin− cells) innervated a significantly greater area of the dorsolateral striatum and led to the improvement of rotational and forelimb asymmetry scores to intact lesion levels when compared to grafts deficient in this cell type (˜15% GIRK2+/calbindin-cells). Nevertheless, most if not all of transplantation studies with stem cell-derived neurons have both populations and show innervation of both A9 and A10 target regions. It would be advisable to control the relative content of GIRK2+ and calbindin+ dopaminergic neurons in TE-NSPs to ensure a therapeutic effect and that the axons innervate the proper striatal targets, a capacity that has been previously identified for dopaminergic subtypes. For example, a different protocol used lentiviral vectors to drive the expression of LMX1A in human ESCs and iPSCs, resulting in over 60% A9 neurons compared to only 10% in controls. Future iterations of TE-NSPs could incorporate protocols that account for differentiation into specific dopaminergic subtypes. Apart from the differentiation protocol, fabrication steps specific to TE-NSPs may also influence cell behavior.

### Example 2

### Materials and Methods

### 1. Synthesis of MeHA and Hydrogel Fabrication

MeHA was synthesized by the esterification of hyaluronic acid (Lifecore Biomedical) with methacrylic anhydride (Sigma, 276685) in cold deionized water for ˜3.5 hr, with the pH maintained at 8.5 throughout with the addition of NaOH. The MeHA solid was obtained after dialysis for 5-7 days and lyophilization. This reaction produced a degree of methacrylation of the hyaluronic acid backbone of ˜40%, as confirmed by 1H-nuclear magnetic resonance spectroscopy. The degree of methacrylation corresponded to the ratio between the area below the two vinyl singlets at ˜5.8 and ˜6.3 ppm and the area below the region corresponding to the sugar ring in the spectra. MeHA solutions were created by adding MeHA for a 3% weight/volume (w/v) to Dulbecco's phosphate buffered saline (DPBS; ThermoFisher, 14190136) having 0.05% w/v Irgacure 2959 (12959; Sigma, 410896) photoinitiator. We characterized the reaction product for its capacity to form hydrogels by light-induced radical polymerization. This was achieved by measuring the storage and loss moduli of the MeHA hydrogel solution over time before and after exposure to 10 mW/cm2 ultraviolet light (UV) using a controlled-stress rheometer (TA Instruments, AR2000) connected to an UV lamp (Excelitas Technologies, OmniCure S1500).

2. Fabrication of MeHA Hydrogel Micro-Columns with an ECM Core

The hydrogel micro-columns used as the encasement for TE-NSPs were fabricated by first inserting an acupuncture needle with an outer diameter (OD) of 500 μm (Lhasa OMS, TC1.50×100) inside a glass capillary tube with an inner diameter (ID) of 973 μm (Drummond Scientific, 1-000-0750). The former and the latter create the lumen and the outer shell of the micro-column, respectively, which has an OD of 973 μm and an ID of 500 μm. The MeHA solution was drawn into the tubes by capillary action, and they were then placed horizontally below the UV lamp and irradiated with 10 mW/cm² light for 5 min. The needles were removed, and the gelled micro-columns within the capillary tubes were pushed out into DPBS using a 20 gauge needle (BD, 305178). The micro-columns were then inspected with light microscopy to remove any that did not form properly and sterilized under UV light for 30-60 min. The micro-columns were cut to the desired length (1-2 cm) under a dissection microscope.

The extracellular matrix (ECM) core of the micro-columns was created by first making a solution of 1 mg/mL rat-tail type I collagen (Corning, 354236) and 1 mg/mL mouse laminin (Corning, 354232) in Neurobasal media (ThermoFisher, 21103049) adjusted to a pH of 7.2-7.4 with 1 N NaOH. After suctioning the DPBS remaining within the lumen of the hydrogel micro-columns, excess ECM solution was added to the lumen to fill it (around 10 μL for a 1 cm long column). The ECM was allowed to polymerize in an incubator at 37° C. for 15 min and, after this period, fresh media was added and floating, excess gelled ECM not inside the columns was removed with forceps. The hydrogel-ECM micro-columns were kept in the media at 37° C. for 1 or 2 days before seeding the neuronal aggregates.

### 3. Aggregation and Culture of Human Dopaminergic Neurons in TE-NSPs

Human iPSC-derived dopaminergic neurons were obtained already differentiated and cryopreserved (iCell DopaNeurons from Fujifilm Cellular Dynamics, R1032), having a purity of 98% MAP2+/NES−, 96% FOXA2+, and 90% TH+ cells as reported by the company. The source somatic cells were obtained from a healthy male of 50-59 years, and the generated iPSCs were differentiated based on the midbrain floor plate protocol. The culture media consisted of iCell Neural Base Medium 1 (Fujifilm Cellular Dynamics, M1010) with 2% Neural Supplement B (M1029), 1% iCell Nervous System Supplement (M1031), and 1% Penicillin-Streptomycin (ThermoFisher, 15140122). After receipt, the cells were thawed by following the protocol suggested by the company. The vial was placed for 3 min in a bead bath at 37° C., after which its contents were slowly transferred to a 50 mL centrifuge tube. Then, after rinsing the vial, 9 mL of culture media were slowly added to the tube and gently mixed by swirling. This cell solution was then moved to a 15 mL tube and centrifuged at 400×g for 5 min. The supernatant was aspirated, the cell pellet was resuspended in 2 mL of media, and the viable cell concentration was quantified with a hemocytometer and Trypan Blue exclusion. New cell solutions were created as needed to form aggregates with the desired number of cells.

The neuronal aggregates were created with custom-made polydimethylsiloxane (PDMS) molds containing nine inverted pyramidal micro-wells. Around 15 μL of cell solution were transferred to each micro-well, and the plate containing the molds was centrifuged at 1500 rpm for 5 min in order to concentrate the cells at the tip of the micro-well. Culture media was added to cover each PDMS mold, and the plate was incubated overnight at 37° C. On the next day, the aggregates were pipetted out of the micro-wells and seeded within one end of the hydrogel micro-columns with fine forceps. In some cases, a microknife was used to make enough space in the ECM to contain the aggregates. For the TE-NSPs presented in this manuscript, we started with aggregates containing 13,269 neurons. Thus, for TE-NSPs containing aggregates with 2.7×10⁴, 5.3×10⁴, and 1.1×10⁵ cells we seeded 2, 4, and 8 aggregates, respectively, which over time merged to become one within the lumen. TE-NSPs were maintained in culture at 37° C. and 5% CO₂, and 75-100% of the media was changed every 3 days.

### 4. Isolation of Rat Embryonic Striatal Neurons

In some cases, an aggregate of striatal neurons was seeded at the end of the hydrogel micro-column opposite to the one containing the dopaminergic aggregate 5 days after seeding the latter. For this study, two striatal aggregates were seeded to have a merged total of 2×10⁵ cells. These striatal neurons were isolated from embryonic day 18 rat pups. Pregnant Sprague-Dawley rats (Charles River) were euthanized by CO₂ exposure and decapitation, and the pups were extracted. Under a dissection microscope, the brains were removed from the heads of the pups and the striatum was cut from each hemisphere, taking care to remove any pieces of cortex. The striata were rinsed with cold Hank's balanced salt solution (HBSS; ThermoFisher, 14175079) and then exposed to accutase (ThermoFisher, A1110501) for 10 min at 37° C., with shaking every 3 min. A Pasteur pipette was used to triturate the tissue until dissolved and then the solution was centrifuged at 200×g for 5 min. The supernatant was removed and the pellet was resuspended in 2 mL of media. This cell solution was utilized to form the aggregates as explained in the previous section.

### 5. Characterization of Growth Length and Rate

TE-NSPs were imaged at 4, 7, 10, 14, 20, and 31 days in vitro using phase contrast microscopy with a Nikon Eclipse Ti-S microscope and QiClick camera integrated with NIS Elements software (Nikon). These images were analyzed to quantify the neurite growth length and rate using ImageJ/Fiji. The neurite growth length corresponded to the distance between the edge of the aggregate and the tip of the longest neurite that could be observed in the lumen. The growth rate was estimated based on the difference between the neurite length at a time point and the previous one relative to the difference in the number of days. The effects of variables such as cell number, time, and/or culture type were assessed using repeated measures ANOVA or a mixed-effects model, and differences between groups were determined with Tukey's or Sidak's multiple comparisons test with the Prism 8 software (GraphPad). In all cases p<0.05 was considered statistically significant.

6. Assessment of Structure and Phenotype with Immunocytochemistry

The structure and phenotype of TE-NSPs were confirmed by immunolabeling these constructs to identify relevant neuronal, dopaminergic, and striatal markers. At terminal time points, TE-NSPs were incubated for 35 min in 4% paraformaldehyde (Electron Microscopy Sciences, 15710) in DPBS to fix the tissue. The constructs were then permeabilized for 1 hr with 0.3% Triton X-100 (Sigma, T8787) in 4% horse serum (ThermoFisher; 16050122). Afterwards, the TE-NSPs were incubated overnight at 4° C. with the primary antibody solution in 4% horse serum. The primary antibodies used in this study were: (1) tyrosine hydroxylase (TH; 1/500, rabbit, Pel-Freez Biologicals, P40101), the enzyme in the rate-limiting reaction of dopamine synthesis; (2) β-tubulin III/Tuj1 ( 1/500, mouse, Sigma, T8578), a neuronal microtubule protein seen in somata and axons; (3) dopamine-and-cAMP-regulated neuronal phosphoprotein (DARPP-32; 1/500, mouse, Santa Cruz Biotechnology, sc-271111), a marker of striatal medium spiny neurons. After rinsing, the constructs were incubated for 2 hr at room temperature with the secondary antibody solution in 4% horse serum ( 1/500, ThermoFisher, donkey anti-mouse 488, donkey anti-rabbit Alexa-568). The nuclei were then stained for 10 min with Hoechst ( 1/10000, ThermoFisher, 33342). After rinsing with DPBS, the full thickness of these constructs was imaged using a Nikon A1RSI laser scanning confocal microscope and the maximum intensity projections were obtained from the z-stacks.

### 7. Quantification of Electrically-Evoked Dopamine Release

TE-NSPs were analyzed at 33-42 days in vitro for electrically-evoked dopamine release using fast-scan cyclic voltammetry. These constructs were placed in a perfusion chamber at 37° C. with an inlet flow of Neurobasal, 2.0 mM L-glutamine (ThermoFisher, 35050061), and 200 μM ascorbic acid (Sigma, A5960) bubbled with a mixture of 95% 02 and 5% CO₂. Stimulation and recording were both done in the same area within the dopaminergic aggregate, axon tracts, or the striatal end. Stimulation was carried out with a bipolar electrode (Plastics One) applying a monophasic+ electrical train of 10 pulses of 5 ms width at 20 Hz and an amplitude of 8 V. Recordings were performed with a carbon fiber electrode (150-200 μm outer fiber length, 7 μm diameter) that had its potential scanned linearly from −0.4 V to 1.2 V to −0.4 V vs. Ag/AgCl at a rate of 400 V/s with a voltammeter/amperometer (Chem-Clamp, Dagan Corporation). Cyclic voltammograms (CVs) were obtained every 100 ms during the recording session using the Demon Voltammetry and Analysis Software (Wake Forest Baptist Medical Center), and the representative current for the session corresponded to the current at the peak oxidation potential of dopamine. For each TE-NSP and recording/stimulation region we obtained the current, the CV at peak release, and a colorplot representing all CVs over time; all these represented the average signal from 3-6 runs within the same area taken 8-12 min apart. Then we processed the data with principal component regression, a chemometric tool combining principal component analysis with inverse least-squares regression, to improve the isolation of the dopamine analyte from CVs that contained other interfering signals. This process was carried out with the Demon software using a training set from awake rats having the CVs corresponding to different known dopamine concentrations and known pHs. The carbon fiber electrodes in use during our recording sessions were calibrated over time, before and after recordings, by determining the evoked current corresponding to injections of 1.5, 3, and 6 μM dopamine hydrochloride (Sigma, H8502). During these calibrations the solvent was deionized water with 126 mM NaCl, 2.5 mM KCl, 1.2 mM NaH₂PO₄—H₂O, 2.4 mM CaCl₂-2H₂O, 1.2 mM MgCl₂-6H₂O, 25 mM NaHCO₃, and 0.4 mM L-ascorbic acid (all from Sigma). The slope of concentration vs. current was used to convert the currents from processed TE-NSP recordings to concentrations. The maximum dopamine release in each case was measured as the difference between the peak concentration during the 2 s after stimulation and the average baseline signal before stimulation. Two-way ANOVA was used to assess the effect of the type of construct (different cell numbers, unidirectional or bidirectional) and recording/stimulation region (dopaminergic aggregate, axon tracts, or striatal end) on the evoked dopamine concentration. Sidak's multiple comparisons test was used to determine differences between groups. In all cases, p<0.05 was considered to be statistically significant.

### Tissue Engineered Nigrostriatal Pathways

Our combinatorial approach centers on engineering the entire nigrostriatal pathway in vitro for implantation into the host brain as a unit to replace/reconstruct the damaged fibers. This platform, which we call the tissue-engineered nigrostriatal pathway (TE-NSP), consists of a hydrogel micro-column with controllable inner/outer diameter and length that has a lumen filled with extracellular matrix (ECM) and seeded on one end with an aggregate of dopaminergic neurons that extends long axon tracts throughout the column. Thus, TE-NSPs mimic the cytoarchitecture of the native nigrostriatal pathway: dopaminergic neurons restricted to one end (the SNpc) projecting axonal tracts (the nigrostriatal fibers) that in turn release dopamine and form synapses along medium spiny neurons (MSN) neurons in the striatum in vivo. The hydrogel encases the engineered dopaminergic tissue, provides necessary physical cues to drive longitudinal axon growth in vitro, and protect it during and after implantation. The ECM ensures neurons have the proper environment to survive and grow axons throughout the lumen.

Herein, we have characterized TE-NSPs fabricated with hyaluronic acid (HA) hydrogels, particularly methacrylated hyaluronic acid (MeHA), as a way to augment the functionality of these constructs and the degree of control we have over their properties. HA hydrogels have been widely used both clinically and in research for various applications. When compared to the bioinert agarose hydrogels, HA is cell-responsive and is one of the main components of the ECM in the brain. There is also a vast trove of research into chemical modifications that can be applied to these hydrogels to enable fine-tuning of physical properties, the presentation of biochemical cues, and the release of growth factors and drugs. Thus, many types of modified HA hydrogels could be explored for TE-NSPs depending on the need and application. The use of these hydrogels may make TE-NSPs more biocompatible, versatile, adaptable to specific design criteria, and optimizable for their purpose of treating PD. Particularly, this may contribute to significantly improved outcomes as a result of better graft survival, preservation of the structure of the engineered axons in vivo, and integration with brain tissue. Moreover, expanding the functionality of the hydrogel micro-column beyond encasement and protection to deliver drugs and other molecules may turn TE-NSPs into a more inclusive approach that addresses other aspects of PD pathophysiology. Using embryonic rat dopaminergic aggregates, we confirmed that MeHA can be utilized to create TE-NSPs that mimic the nigrostriatal pathway in terms of structure, phenotype, and dopamine release and that can be implanted and survive in the rat brain. Notably, MeHA-encased TE-NSPs featured faster and longer axon growth and elicited a lesser astrocyte and microglia response in the brain after 6 weeks in vivo when compared to traditional 1% agarose encasement.

### Advantages of Preformed Neural Networks and TE-NSPs

TE-NSPs have been designed to improve upon and circumvent the limitations of both conventional PD interventions and approaches centered on cell replacement and pathway reconstruction. On the first front, TE-NSPs directly address the underlying causes of PD by replacing the lost dopaminergic neurons in the SNpc and by reconstructing the cytoarchitecture of the dopaminergic fibers that encompass the nigrostriatal pathway. In doing so, TE-NSPs target both ends of the nigrostriatal circuit and may thus release dopamine in the striatum in a biologically- and temporally-controlled manner in response to their interaction with the host brain and with the spatial specificity provided by targeted innervation and synaptic communication. This contrasts with L-DOPA, other drug therapies, and electrical stimulation through DBS, which indirectly treat PD by managing symptoms in a nonspecific manner and tend to cause debilitating off-target effects.

In the case of biomaterials- and/or cell-based strategies, research efforts and clinical trials have concentrated on the injection of suspensions of dopaminergic neurons into the striatum to provide innervation in situ, overlooking the loss of cells in the SNpc and their nigrostriatal fibers. On the other hand, TE-NSPs involve implanting an entire nigrostriatal pathway substitute that matches the actual neuroanatomy and may synaptically integrate with targets around the SN and in the striatum. Having the dopaminergic neurons and axons in their proper location may restore the integration with other regions associated with the motor circuit, which should ultimately augment the therapeutic benefit of these constructs over cell replacement alone. Furthermore, TE-NSPs have a protective hydrogel encasement that may promote better cell survival during and after implantation and greater cell retention in the intended anatomical areas, in contrast with the low cell survival and the possibility for off-target cell presence observed with traditional implants.

Studies looking at ways to repair nigrostriatal fibers have focused on injecting neurons in the SN and either passively allowing them to extend axons in vivo or actively guiding their growth by making the local microenvironment amenable to growth and by implanting other cells along the pathway or in the striatum. Regardless, as previously mentioned, the extent of this in vivo growth would not be enough for the distances required in humans and oftentimes the resulting innervation lacked specificity. TE-NSPs ultimately avoid needing axons to grow through the perilous environment in vivo, as these constructs are implanted as a unit after growing axons with the required length, diameter, and density in vitro. This complete biofabrication in vitro bypasses the need for precise growth conditions, signaling cues, or axon pathfinding along existing pathways in the brain microenvironment that are necessary for extensive growth and innervation. The use of a tubular hydrogel for growing and encasing the axons may also help ensure that the growth is restricted to occur in a direction similar to the native pathway and that the innervation is spatially targeted to the striatum after implantation. The hydrogel may also be further modified to accommodate the more curved trajectory of the human nigrostriatal pathway. With TE-NSPs we can ideally control the phenotypic distribution in the aggregate and the kinds of projected axons in order to guarantee the type of striatal reinnervation that maximizes motor improvements. We can also exploit fabrication happening entirely in vitro to fully validate the final structure and function of the constructs before implantation, specifically in terms of axon growth as a function of time, viability, expression of mature/functional dopaminergic markers, and dopamine release. Thus, TE-NSPs can be a more reproducible, controllable, and consistent alternative than other less reliable methods that rely on the correct circumstances presenting themselves in vivo.

As previously suggested, the presence of the hydrogel encasement can also expand the capabilities of TE-NSPs to tackle the precise drivers of neurodegeneration. For example, the hydrogel micro-column could also be employed to deliver and release neurotrophic factors, such as GDNF, neurturin, and BDNF, that have been shown to possess neuroprotective effects on midbrain dopaminergic neurons and functional benefits in animals models and clinical trials after injection/infusion, viral-induced expression, or delivery in combination with cells. Similarly, the hydrogel could be leveraged to target other components of PD pathology, such as α-synuclein aggregation and mitochondrial dysfunction, as has been investigated through the delivery of heat-shock proteins, vectors to reduce α-synuclein gene transcription or expression, drugs to increase clearance of α-synuclein, among other approaches. Thus, our TE-NSP technology features several key benefits over existing and nascent approaches to treat PD. By leveraging different approaches taken thus far, inspiring its design on neuroanatomy and PD pathophysiology, incorporating tissue engineering, featuring an expansive degree of control and versatility, and being fully fabricated, optimized and assessed for quality in vitro before use, we believe TE-NSPs will be the ideal method to repair nigrostriatal fibers.

### Effect of Cell Dose on Axon Growth

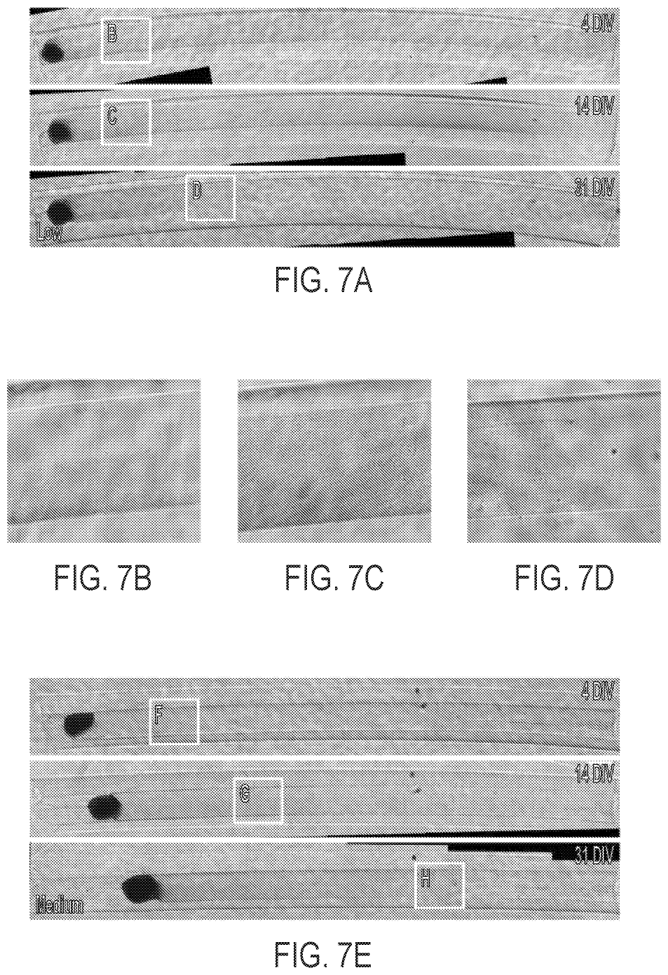

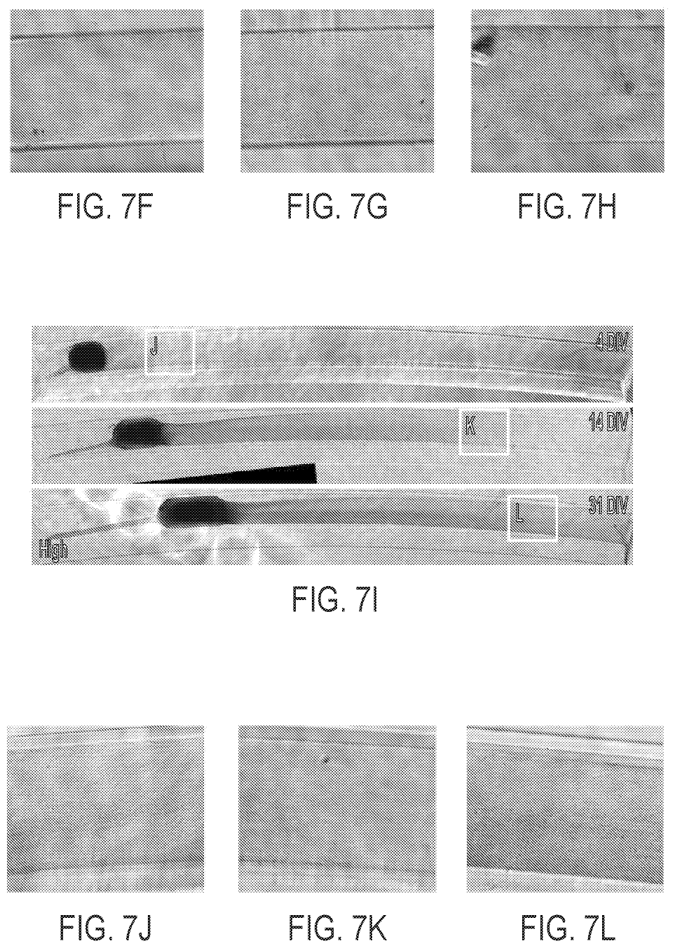

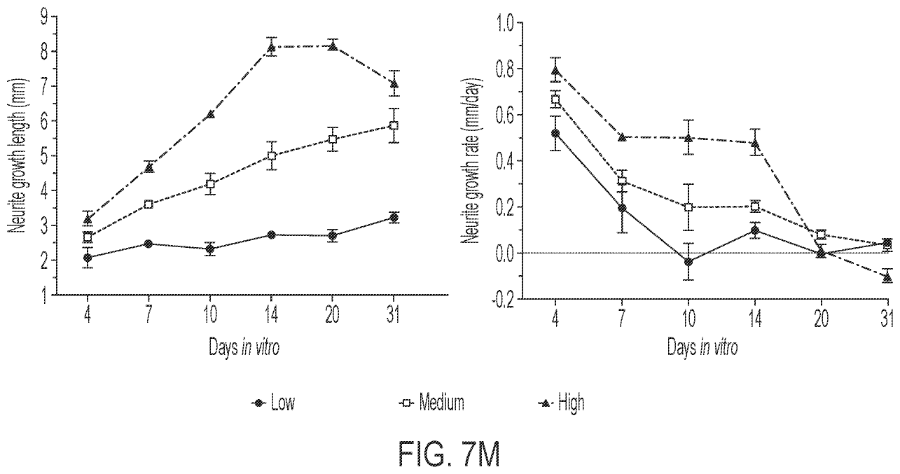

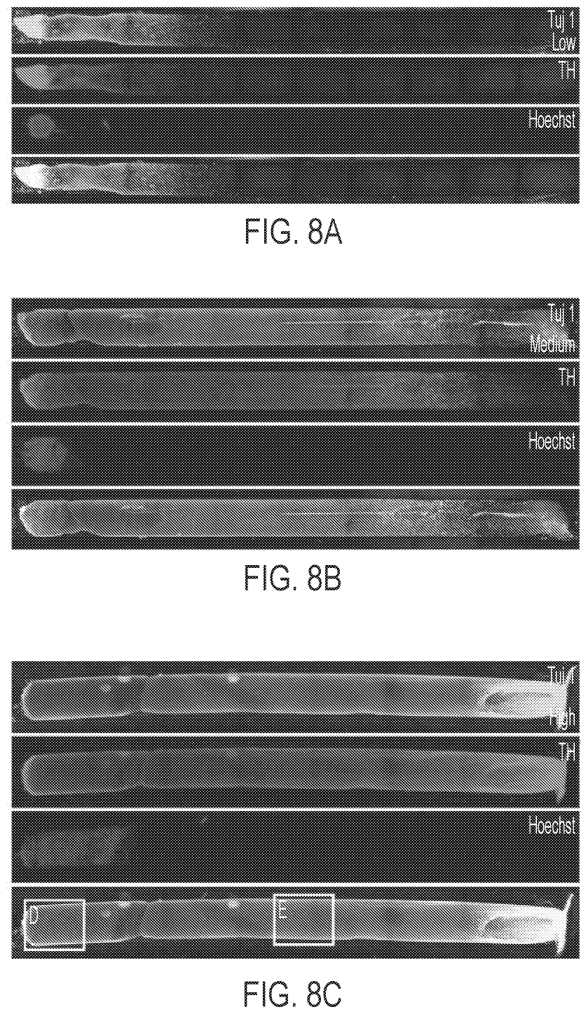

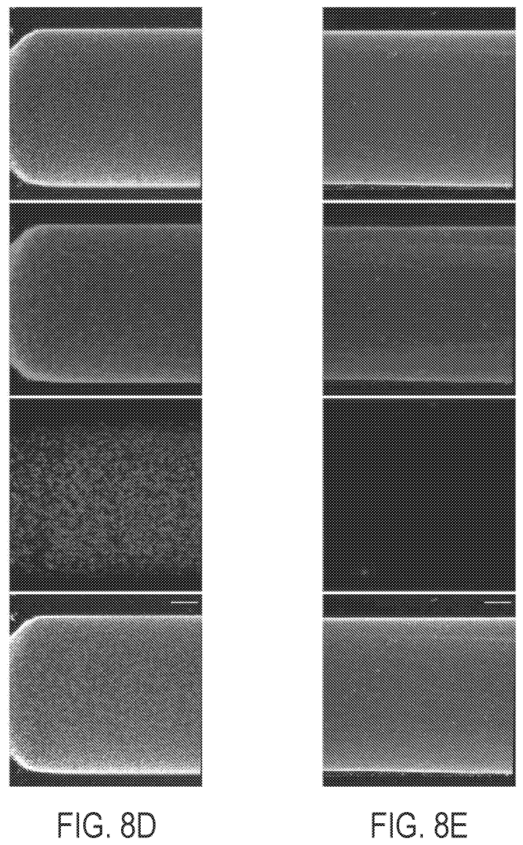

First, we characterized the growth length and rate of neurites projected from human dopaminergic aggregates within 1 cm-long micro-columns as a function of cell dose/number. We used aggregates having a total of 2.7×10⁴, 5.3×10⁴, or 1.1×10⁵ neurons. According to the manufacturer, these neurons were 90% TH+; therefore, these aggregates did contain mostly dopaminergic neurons. Based on phase contrast imaging, it could be qualitatively determined that with higher cell numbers the neurites grow longer distances and have greater density (FIGS. 7A, 7E, 7I). The latter is observed more clearly when magnifying areas of the lumen of the micro-columns (FIGS. 7B-7D, 7F-7H, 7J-7L). Statistical analysis suggested a significant role for time and cell number in both neurite growth length and rate (p<0.0001; mixed-effects model). Length quantification showed that constructs with 2.7×10⁴, 5.3×10⁴, or 1.1×10⁵ cells reach average growth peaks of 3.22±0.16, 5.85±0.48, and 8.17±0.17 mm, respectively, after 1 month in culture (FIG. 7M). From 7 to 31 days most groups were significantly different from each other using Tukey's multiple comparisons test. It is expected that TE-NSPs with high cell numbers would grow even longer, but growth was restricted because the axons reached the end of the micro-columns and because the available space for growth was reduced over time as a result of aggregate movement. Growth rates were highest at 4 days and decreased over time until reaching values close to zero because of neurites stopping their growth or reaching the end. Of note, the higher cell number group had a more consistent growth rate of around 0.5 mm/day from 7 to 14 days and significantly different rates at 10 and 14 days when compared to the lower number group (FIG. 7M). Confocal imaging of immunolabeled TE-NSPs confirmed the longer growth and higher axon density of TE-NSPs with 1.1×10⁵ cells (FIGS. 8A-8C). Moreover, the images confirmed the dopaminergic TH+ phenotype of the aggregate and axons of these constructs (FIGS. 8D-8E). The data here show that aggregates with higher cell numbers are needed to achieve the desired growth lengths and at a faster rate. The greater number of cells may directly result in more axons being projected and/or these higher cell densities may benefit cell viability and health. Still, we anticipate this effect to reach a plateau given the mass transfer limitations for larger tissues. The data confirm that we can fabricate TE-NSPs having around 100,000 neurons, which is in the range of clinically relevant numbers of dopaminergic neurons for implantation. We still require to test even greater numbers of cells to determine the maximum number of cells that a single TE-NSP can hold before detrimental effects on viability and growth.

### Effect of Cell Dose on Dopamine Release

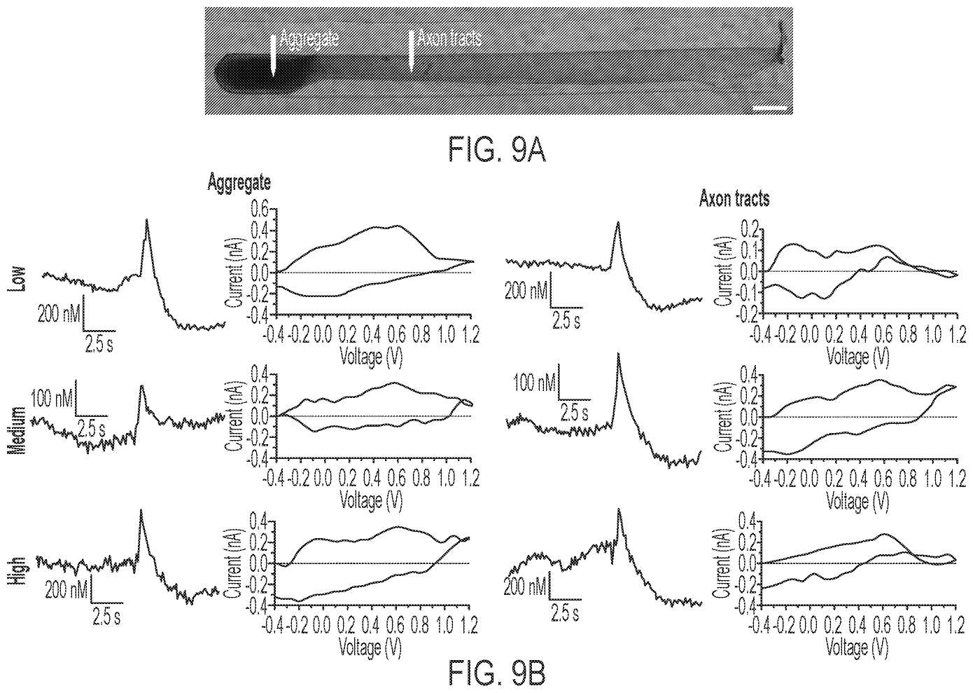

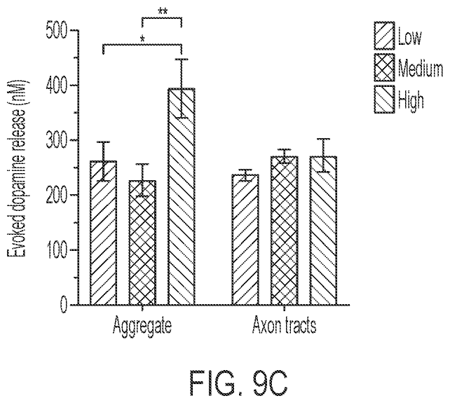

A key necessity for any pathway reconstruction strategy is that grafts must be capable of releasing dopamine. Thus, we tested human-scale TE-NSPs for electrically-evoked dopamine release using fast scan cyclic voltammetry (FSCV). We transferred individual constructs to a recording chamber where stimulation and carbon fiber electrodes were both placed either within the dopaminergic aggregate or the axon tract region (FIG. 9A). The concentration of dopamine released after stimulation was determined from the current detected by the carbon fiber electrode after oxidation of dopamine around 0.6 V and from calibration curves of known concentrations of dopamine. It must be noted that FSCV measures local dopamine concentrations in close proximity to the carbon fiber tip and not what is being released by all cells. TE-NSPs in all cell number groups responded to stimulation by releasing dopamine in the aggregate and axonal tracts, as observed by the peaks in the concentration traces and the characteristic peak in the cyclic voltammogram around 0.6 V (FIG. 9B). The presence of dopamine release in the aggregate points to the existence of somatodendritic release in these constructs, while the recordings in the axons show evidence of the more traditional axonal release at presynaptic terminals. This suggests that these constructs could release dopamine from both ends when implanted and after integration, thus regulating the entire nigrostriatal circuit. Quantification and statistical analysis of peak dopamine release as a function of region and cell number indicated a significant effect of cell number (p=0.0366; two-way ANOVA). In particular, the mean dopamine release in the aggregate was 393.19±54.66 nM for the highest cell number group, which was significantly different from the lower (261.53±35.49 nM; p=0.0343) and medium (226.48±28.91 nM; p=0.0087) groups. There were no differences in the axon tract region, with concentrations fluctuating around 236 to 270 nM. These results reflect a possible role for cell number in the aggregates in dopamine release from human-scale TE-NSPs.

### Integration Between TE-NSPs and Striatal Neurons

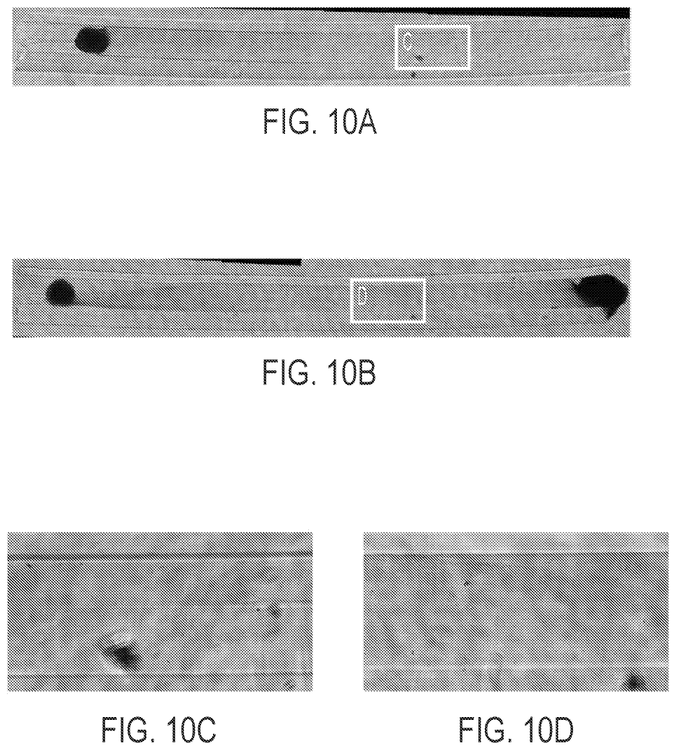

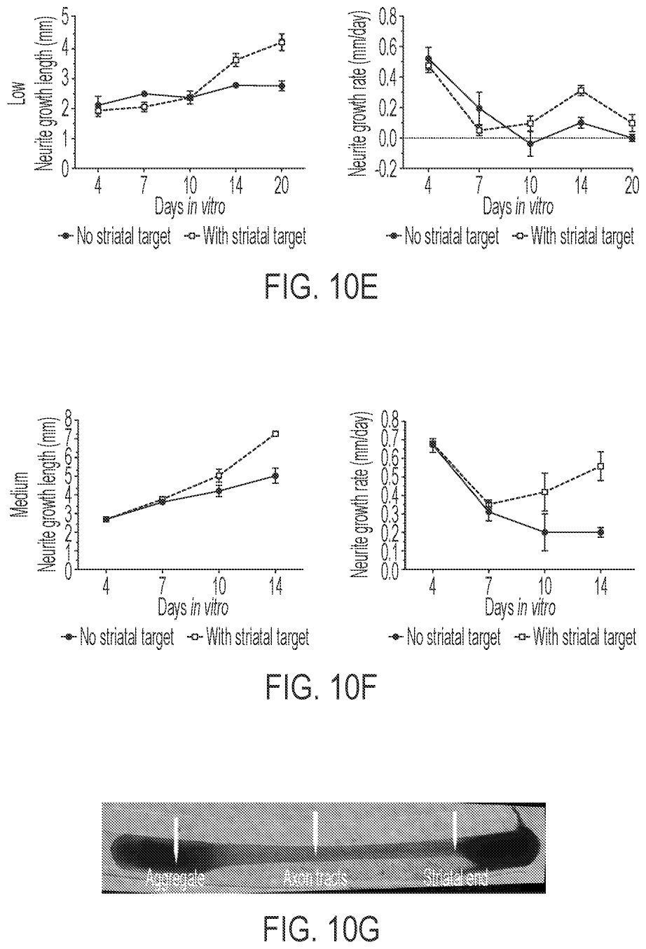

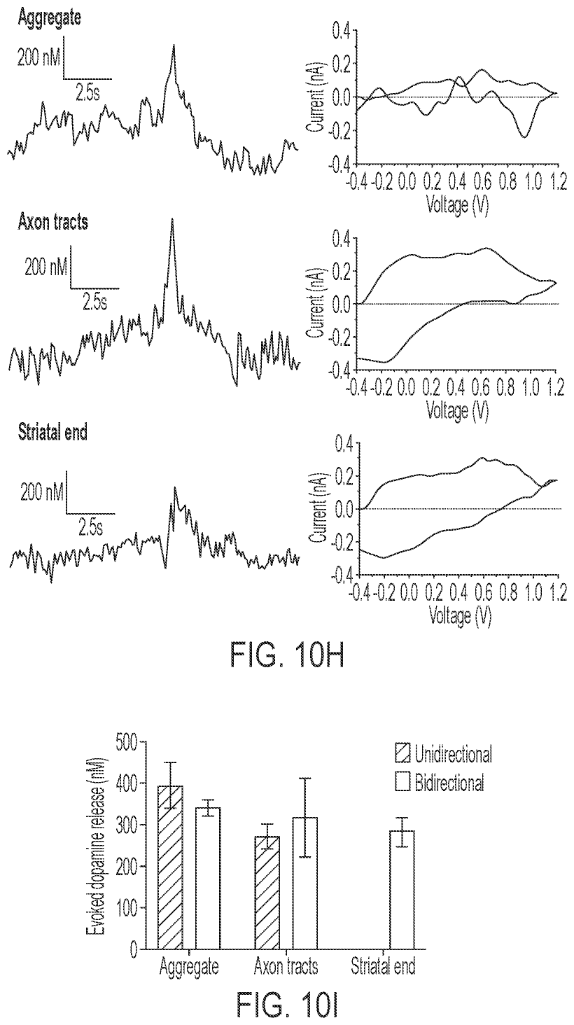

We sought to simulate the connectivity between dopaminergic axons and their striatal targets seen in the native nigrostriatal pathway within TE-NSPs. Thus, we fabricated human-scale TE-NSPs with an aggregate of around 2×10⁵ striatal neurons, isolated from E18 rat pups and seeded 5 days after the dopaminergic aggregate within the end of the micro-column opposite to the dopaminergic one. Moreover, we sought to examine the effect of the presence of these cells on growth and dopamine release. On the first front, phase contrast images showed a qualitative improvement on axon growth length and density at the same distance from the dopaminergic aggregate in bidirectional constructs having the striatal aggregate (FIGS. 10A-10D). This is displayed in the clear separation of growth lengths and rates in bidirectional constructs over unidirectional ones starting around 10 days of culture, in cases with 2.7×10⁴ cells (FIG. 10E) and 5.3×10⁴ (FIG. 10F) cells in the dopaminergic aggregate. The effect was strongest in the medium number group, with two-way ANOVA yielding a significant effect for culture type on lengths (p=0.0104) and growth rates (p=0.0028) only in this group. As an example, at 14 days neurite lengths were 5.01±0.40 mm and 7.24±0.05 mm (unidirectional vs. bidirectional; p=0.0415), while the growth rates were 0.20±0.03 mm/day and 0.56±0.08 mm/day (unidirectional vs. bidirectional; p=0.0596). The case for 1.1×10⁵ cells was not included because growth was too similar and fast in both cases to see a difference before the axons reached the edge of the striatal aggregate or the micro-column. Overall, the data indicate that the presence of a striatal target can improve the growth of human dopaminergic axons. This effect is delayed relative to the day in which the striatal target was added, as the effects could be seen starting on day 10 and the cells were seeded on day 5. This could reflect the time needed for the striatal target to habituate and start to present or release signaling factors benefitting dopaminergic growth or the time required for axons to grow enough to be at a certain threshold distance from the striatal end. We also tested bidirectional TE-NSPs for dopamine release using FSCV, recording and stimulating in the dopaminergic aggregate, axon tracts, and axons at the edge of the striatal aggregate (FIG. 10G). We utilized constructs with the highest number of cells in the dopaminergic axons to ensure complete integration between the two aggregates. We were able to record electrically-evoked dopamine release in all three regions (FIG. 10H). Of note, the dopaminergic axons at the striatal end could be stimulated to release on average 280.16±35.42 nM, which was similar to the 270.69±30.45 nM in the axon tracts of unidirectional constructs (FIG. 10I). There was no effect for culture type (p=0.9432) and region (p=0.2447) after two-way ANOVA, and there were no significant differences in release between groups in any region. Importantly, these studies showed that bidirectional TE-NSPs are functional and that dopaminergic axons integrated with striatal neurons can release dopamine, which would be essential for the success of these constructs in restoring striatal dopamine input.

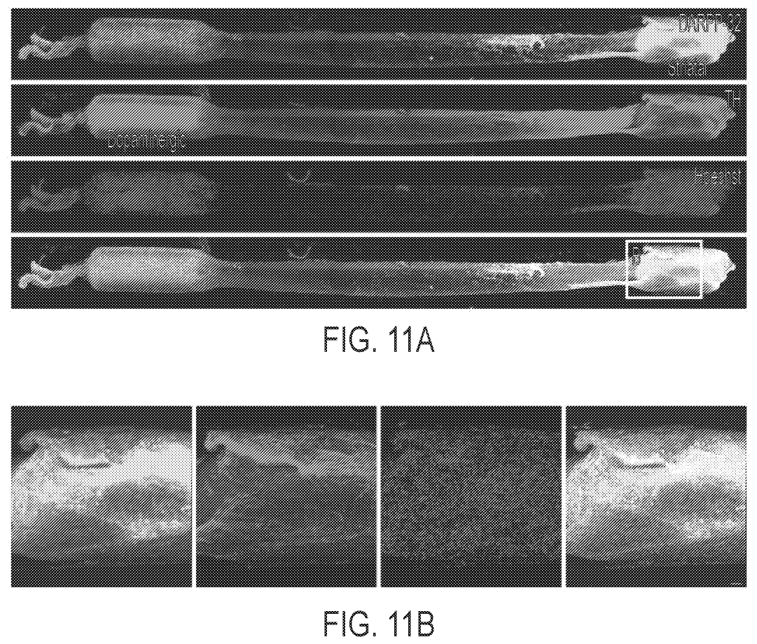

Bidirectional, human-scale TE-NSPs with a striatal aggregate were also stained for dopaminergic (TH) and striatal MSN (DARPP-32) markers and imaged to evaluate the cytoarchitecture of the engineered tissue and physical connectivity between the aggregates. Both phenotypes were observed, with TH expressed strongly in the dopaminergic aggregate and axon tracts and DARPP-32 mainly seen in the striatal aggregate, adjacent neurites (assumed to be dendrites), and migrating neurons (FIG. 11A). Furthermore, a magnification of the striatal aggregate showed a significant presence of TH+ dopaminergic axons exhibiting several patterns of arborization and innervation density (FIG. 11B). These axons should theoretically form synaptic connections with the DARPP-32+ MSNs of the striatal aggregate, demonstrating the capacity of these human-scale TE-NSPs to innervate and connect with striatal neurons.

### Example 3: In Vitro Biofabrication and Characteristics of Human Stem Cell Derived TE-NSPs Encased in MeHA Microcolumns

This Example describes the in vivo biomaterial response and the in vitro biofabrication of human stem cell derived TE-NSPs encased in methacrylated hyaluronic acid (MeHA) microcolumns, and characteristics of the human TE-NSPs.

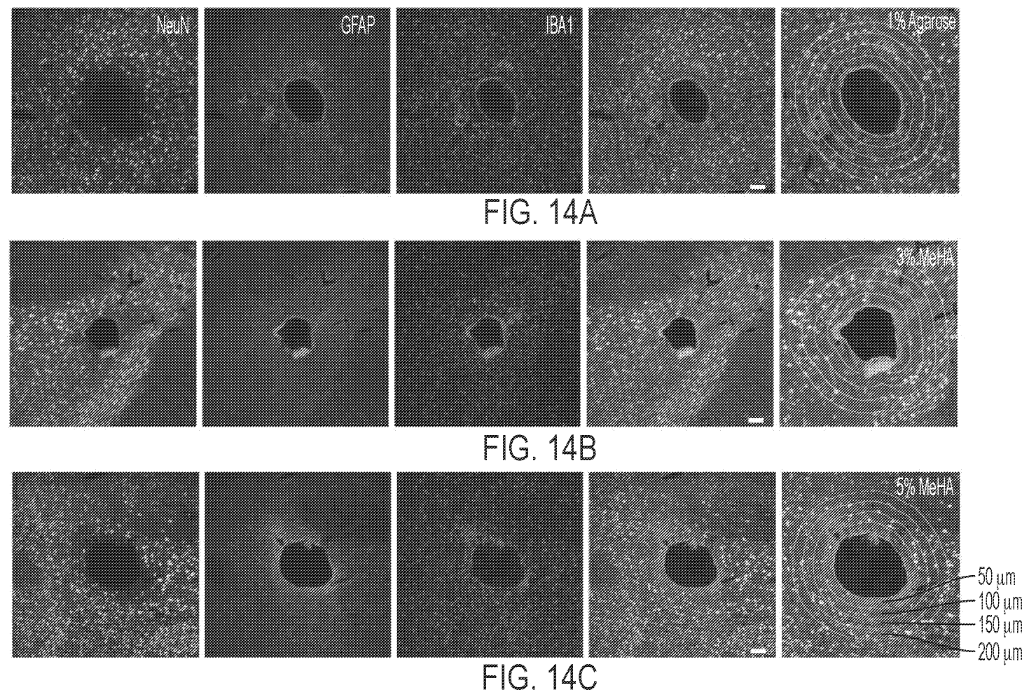

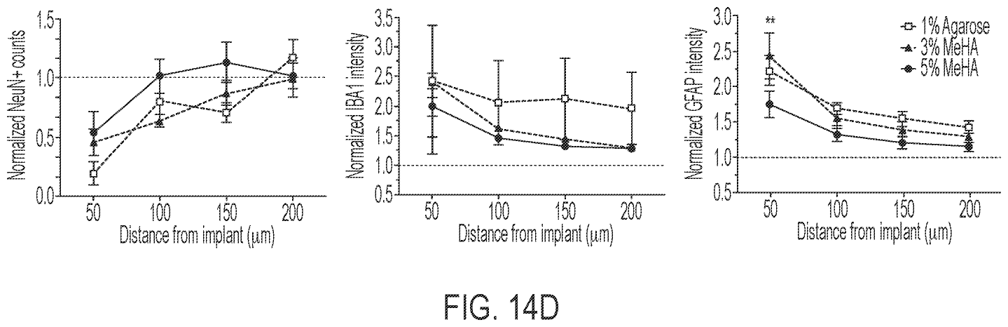

To evaluate the rat brain response to implantation of MeHA columns along the nigrostriatal pathway, acellular hydrogel micro-columns fabricated with % agarose (n=3), 3% MeHA (n=5), and 5% MeHA (n=5), respectively, were implanted along the nigrostriatal pathway of athymic rats for 6 weeks. The host brain response was then assessed. For NeuN+ counts, an increase in counts was observed with increasing distance from the implant (FIG. 14D left panel). IBA1 and GFAP intensity depended on the type of material used and distance from the implant, with increasing distance from the implant showing reduced intensity (FIG. 14D middle and right panels).

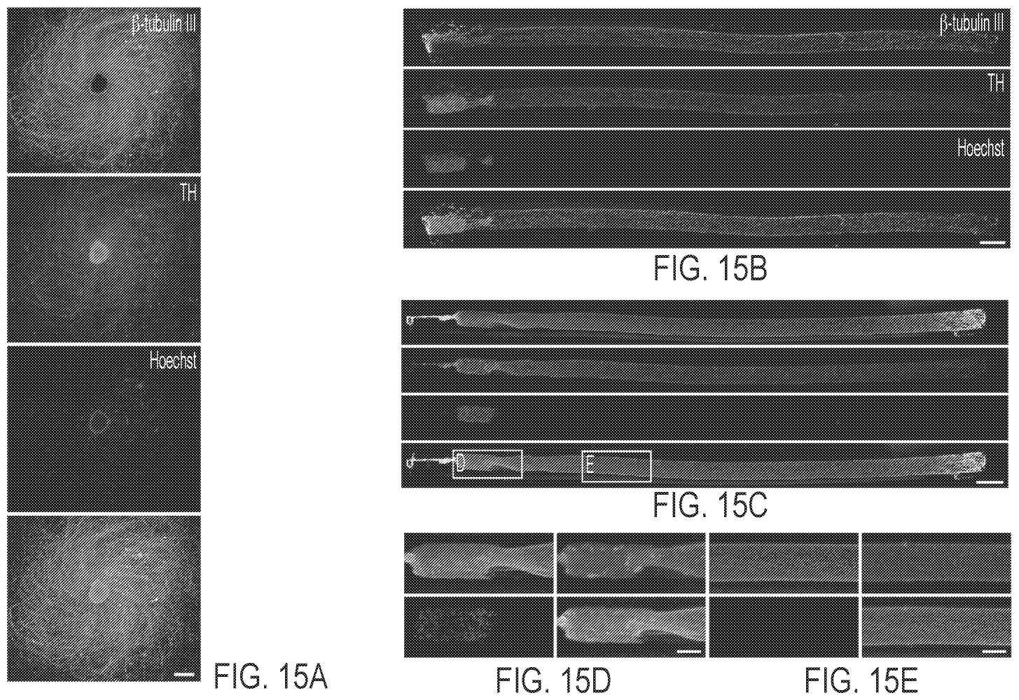

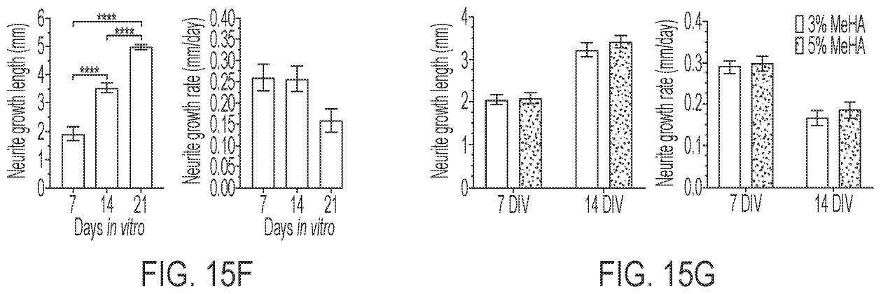

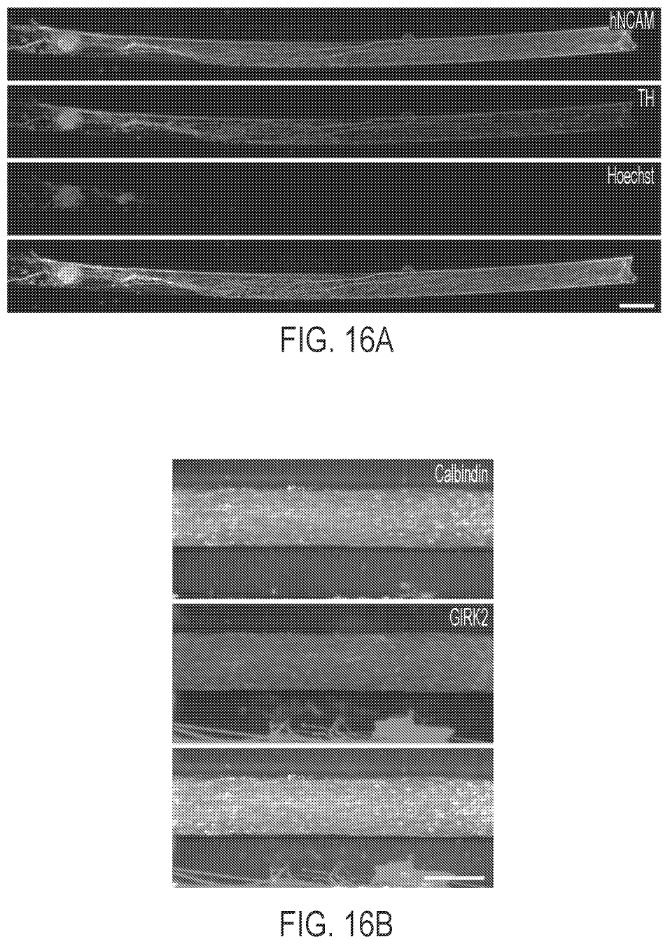

Next, the in vitro cytoarchitecture and growth profiles of rat-scale human TE-NSPs encased in MeHA hydrogel was evaluated. The human iPSC-derived dopaminergic neurons displayed aggregate and axonal tracts as well as expression of dopaminergic neuron markers (FIGS. 15A-15E). Additionally, the human TE-NSPs demonstrated neurite growth (FIG. 15F). The growth length of the neurites increased with time in vitro, with Day 14 neurites showing about double neurite growth length compared to Day 7, and Day 21 neurites showing about 2.5 fold increase in neurite growth length compared to Day 7. The effect of biomaterial composition on growth length and rates was also characterized using TE-NSPs made with 3% MeHA or 5% MeHA hydrogel micro-columns. As demonstrated in FIG. 15G, there was a time dependent increase in neurite growth length (compare 7 days in vitro [DIV] to 14 DIV), but the type of biomaterial did not have a significant effect on neurite growth length or neurite growth rate. The human TE-NSPs encased in 3% MeHA hydrogels also displayed expression of human neural and midbrain dopaminergic phenotypes (FIGS. 16A-16B).

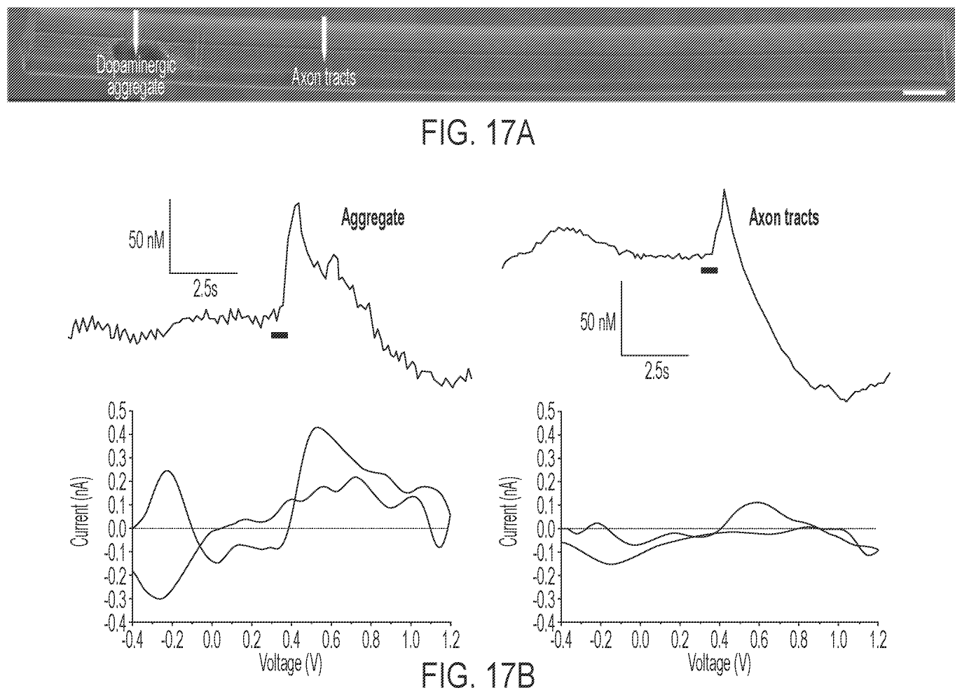

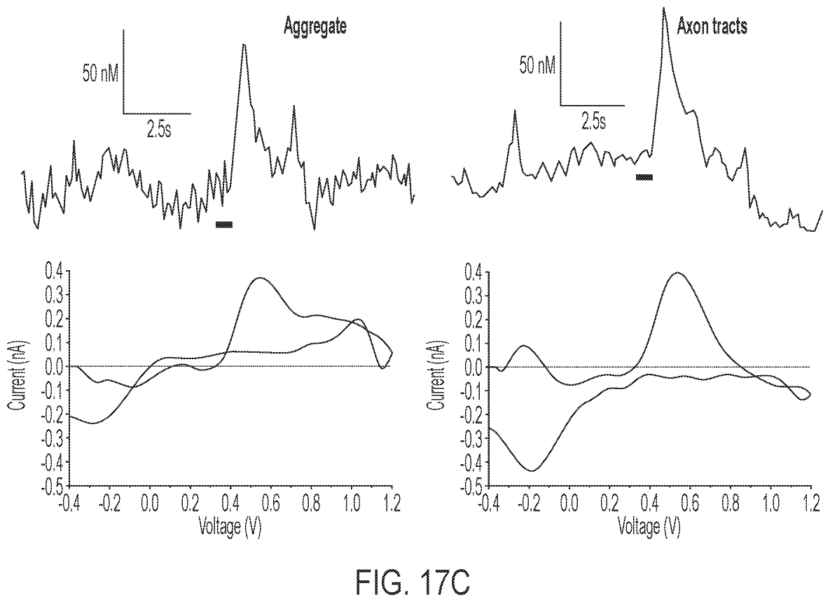

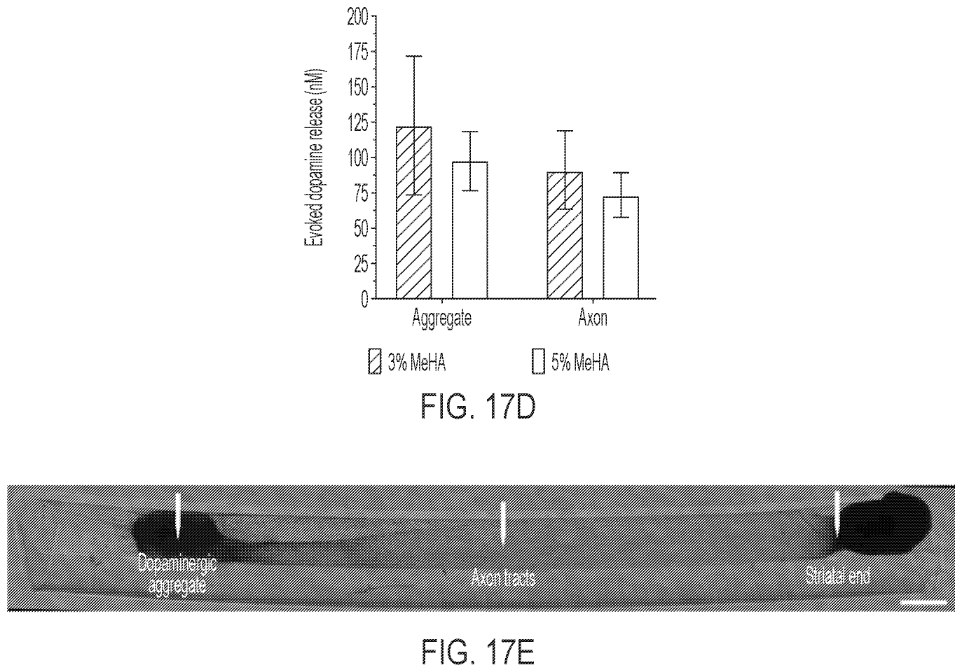

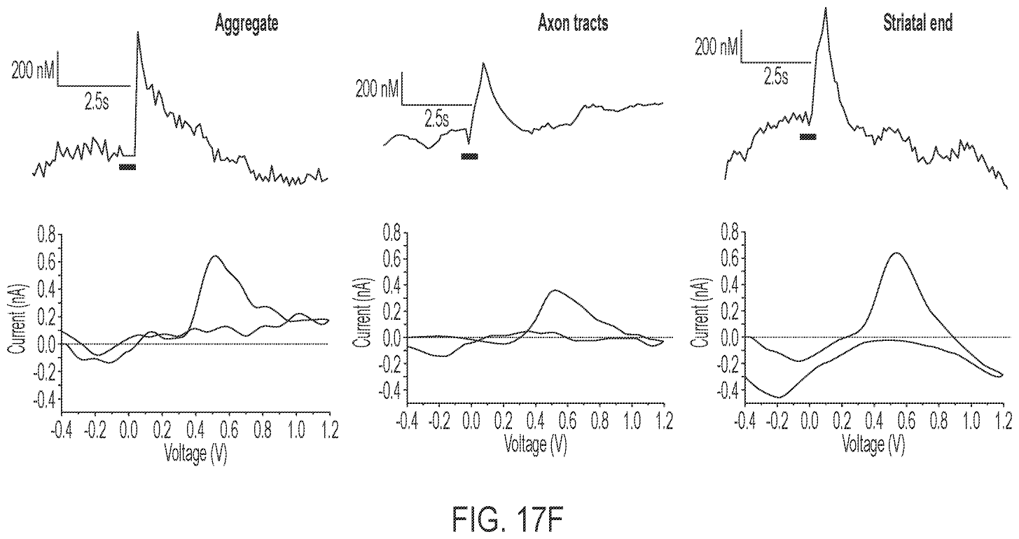

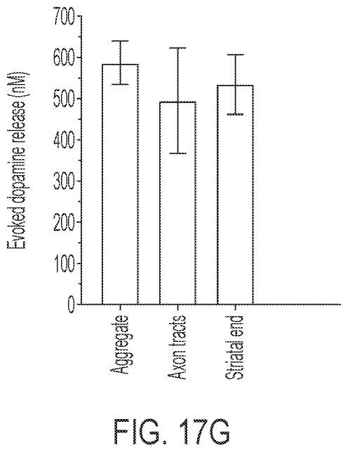

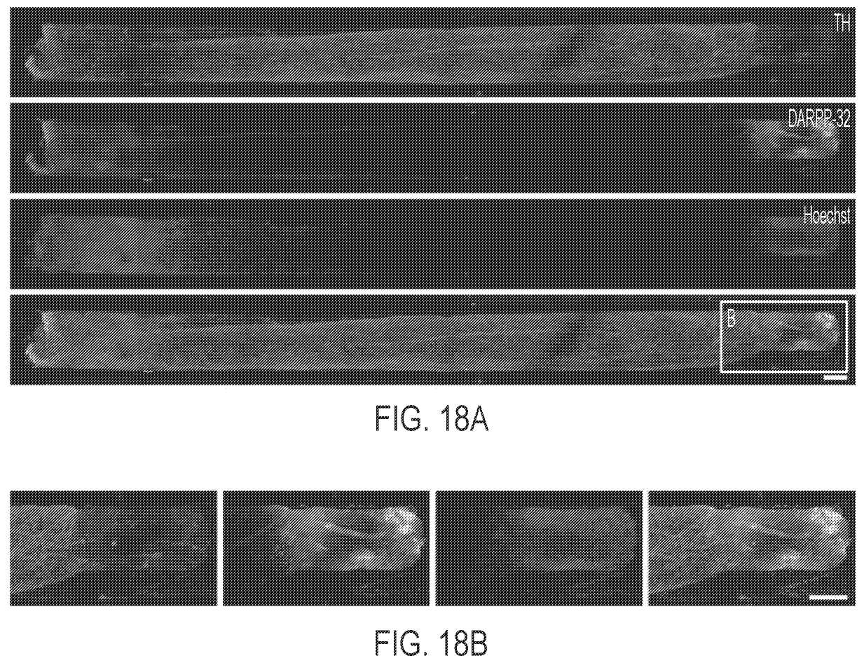

The human TE-NSPs tested evoked dopamine release in vitro. In experiments using rat-scale human TE-NSP with a dopaminergic aggregate at one end (see FIG. 17A), evoked dopamine release was observed both in aggregate and in axon tracts (FIGS. 17B-17D). The data suggests that the recording/stimulation region and the biomaterial do not have a significant effect on dopamine concentration. When human TE-NSPs with an aggregate of rat embryonic striatal neurons at the end opposite from the dopaminergic aggregate within a 3% MeHA micro-column with an ID of 500 μm were used (see FIG. 17E), higher levels of evoked dopamine release was observed in dopaminergic aggregate, axon tracts, and axons in the striatal end (FIGS. 17F-17G). No significant difference in the concentration of evoked dopamine was observed based on the recording/stimulation region. The human TE-NSPs with an aggregate of rat embryonic striatal neurons also demonstrated dopaminergic innervation of the striatal aggregate (FIGS. 18A-18B).

In summary, the data in this Example shows that in vitro biofabricated human stem cell derived TE-NSPs encased in MeHA microcolumns release dopamine, display dopaminergic phenotypes, and promote neurite growth.

### Example 4: In Vivo Efficacy of Human Stem Cell Derived TE-NSPs Encased in MeHA Microcolumns

This Example describes the in vivo implantation and efficacy of human stem cell derived TE-NSPs encased in MeHA microcolumns in athymic rat models of Parkinson's Disease (PD). The implanted TE-NSPs had neurons that persisted long-term in vivo (for at least 6 months), were able to innervate out of the construct and into the striatum, and demonstrated physiological functions.

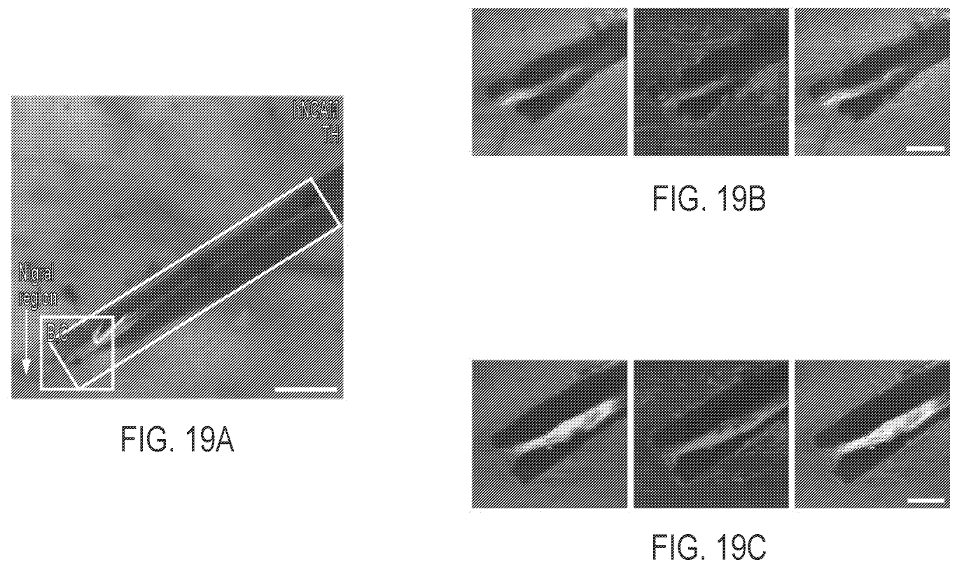

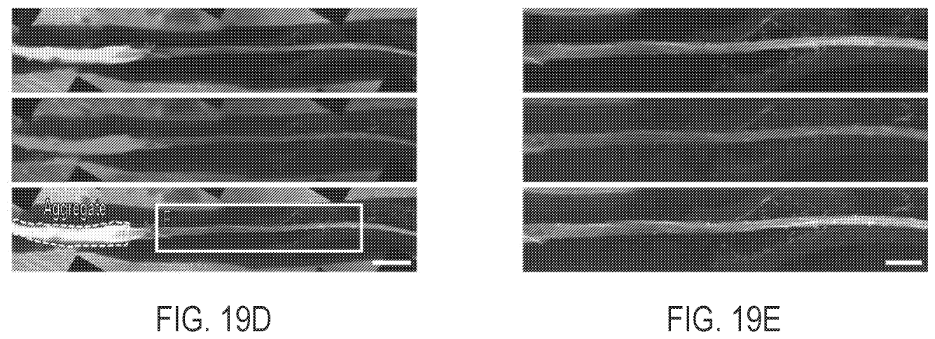

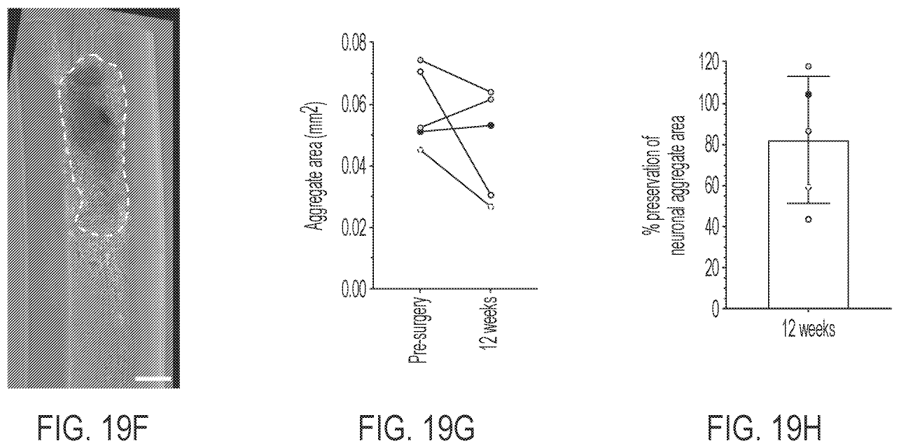

Human TE-NSPs were implanted in a rat model of PD. At 12 weeks post implantation, the animals were sacrificed and brain sections were obtained for histological assessments. As shown in FIGS. 19A-19D, ingrowth of host TH+/hNCAM− cells into the construct and outgrowth of TE-NSPs into the nigral region of the host was observed. Histological assessment of the TE-NSPs also showed that the axon tracts were maintained 12 weeks post-implantation (FIG. 19E). The area of the aggregate in the TE-NSPs implanted in the rats was also assessed. FIGS. 19G-19H demonstrate that there was about 80% preservation of neuronal aggregate area in the TE-NSPs at 12 weeks post-implantation.

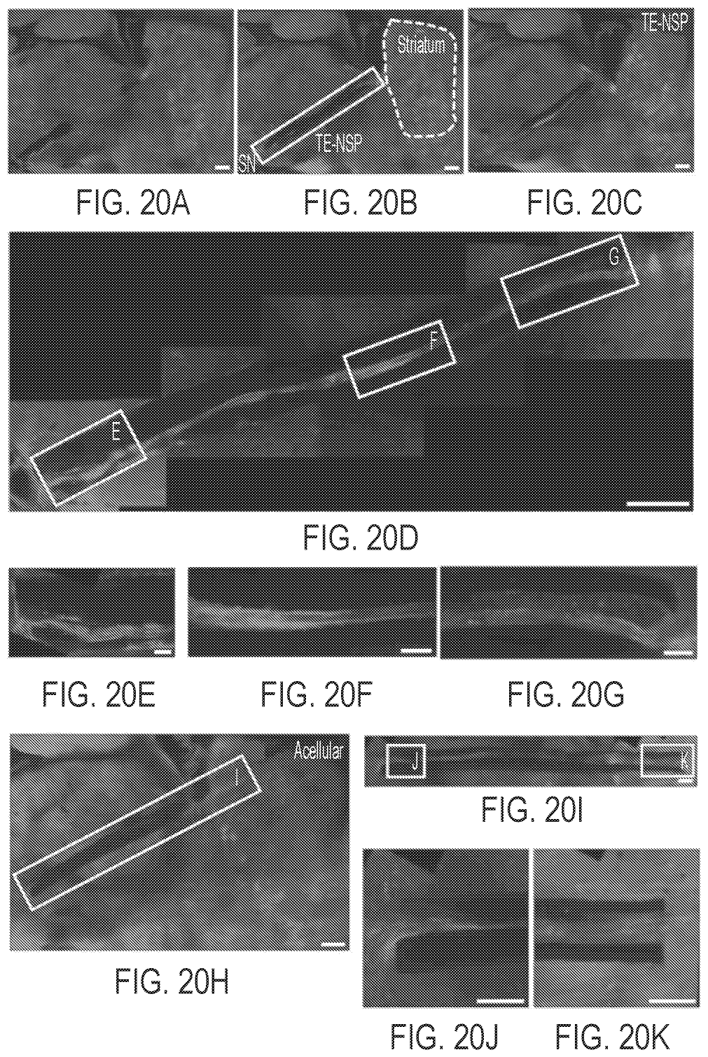

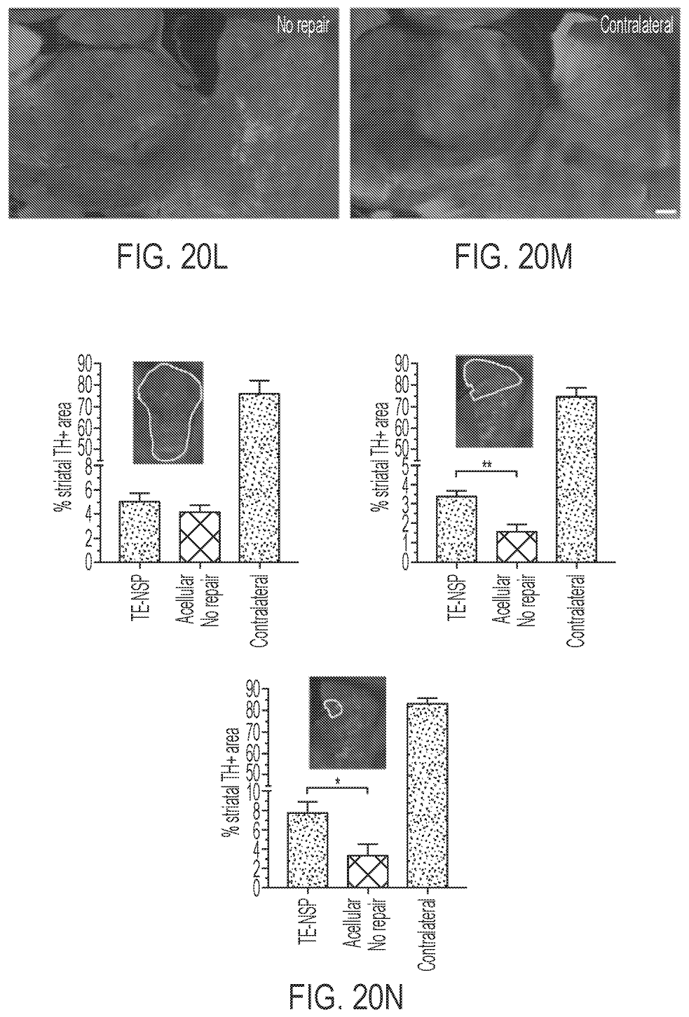

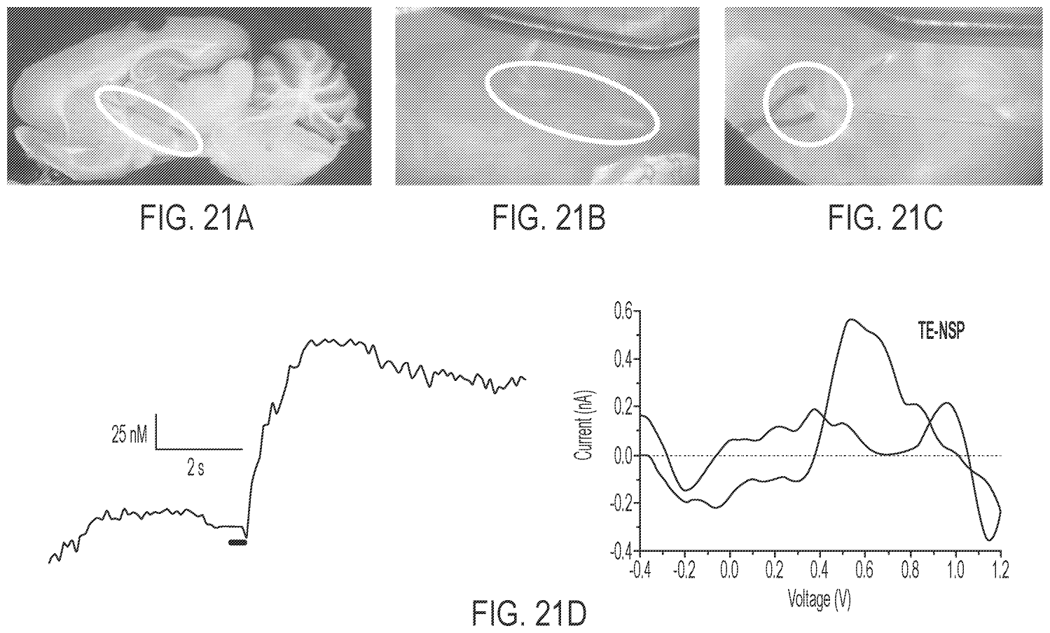

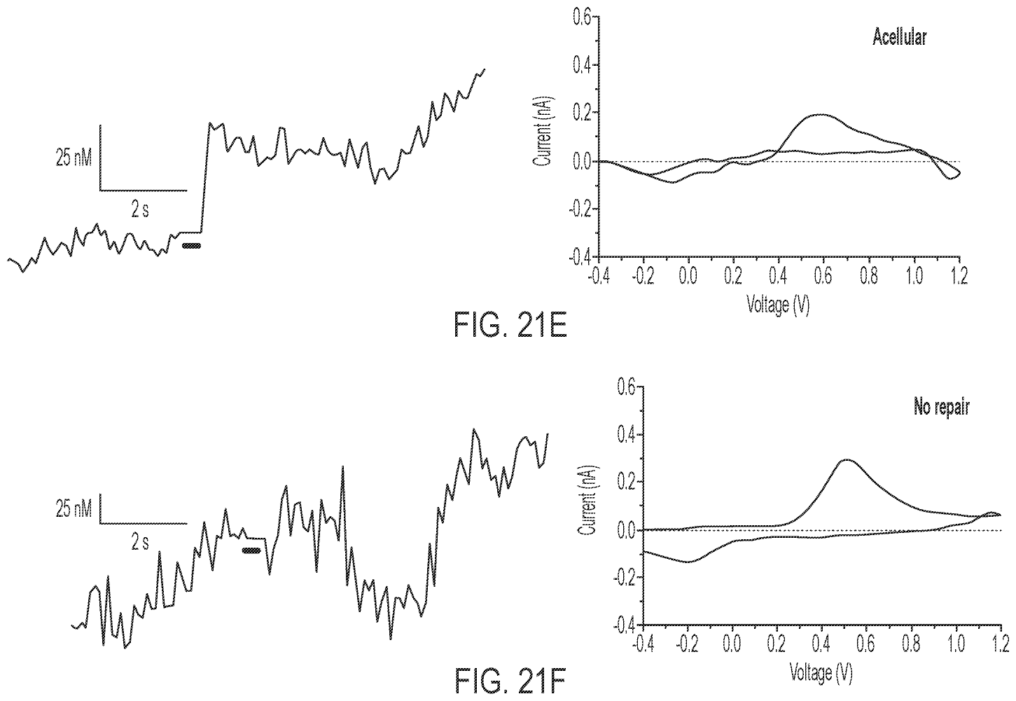

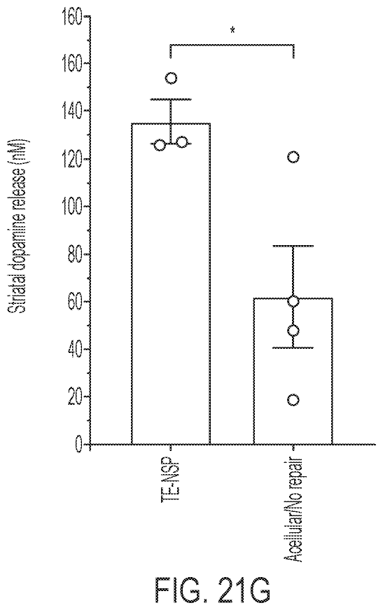

Axon preservation and striatal innervation was also observed in this rat model upon implantation of TE-NSPs. At 12 weeks post-implantation, preserved inner axon tracts and axons ending near the striatum were observed (FIGS. 20A-20G). Implantation of human TE-NSPs resulted in striatal reinnervation compared to implantation of control acellular micro-columns (FIG. 20N). Striatal reinnervation with the human TE-NSPs was observed across all three regions of interest (ROI)s: entire striatum (FIG. 20N, top panel), dorsal striatum (FIG. 20 center panel) and striatal edge near the end of the implanted constructs (FIG. 20N, bottom panel). Next, striatal dopamine levels were evaluated in lesioned rats after TE-NSP implantation. FIGS. 21D-21G demonstrate that striatal dopamine release was highest (more than 2-fold higher) in the striatum of rats implanted with the TE-NSPs as compared to control animals implanted with acellular micro-columns or with no repair.

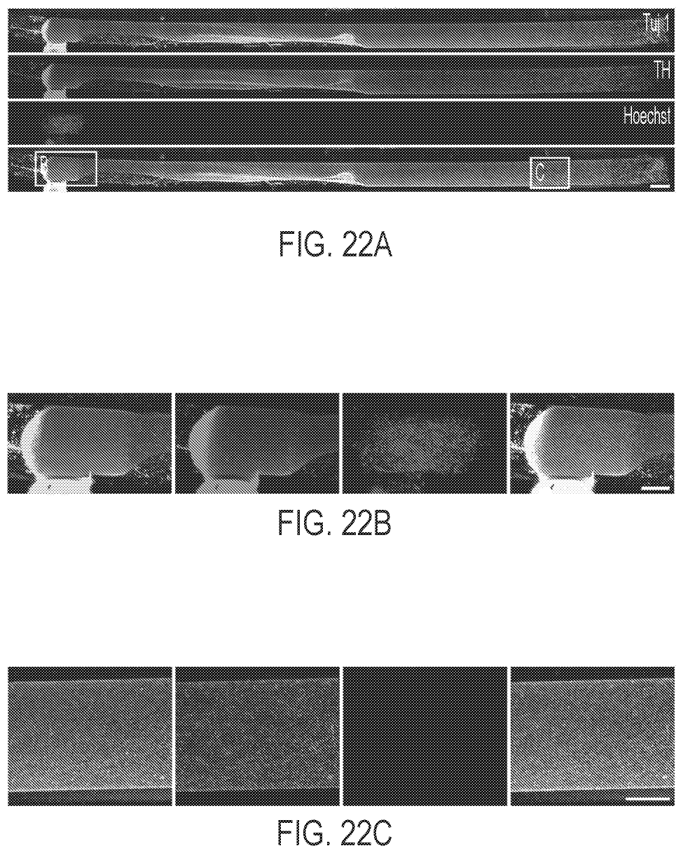

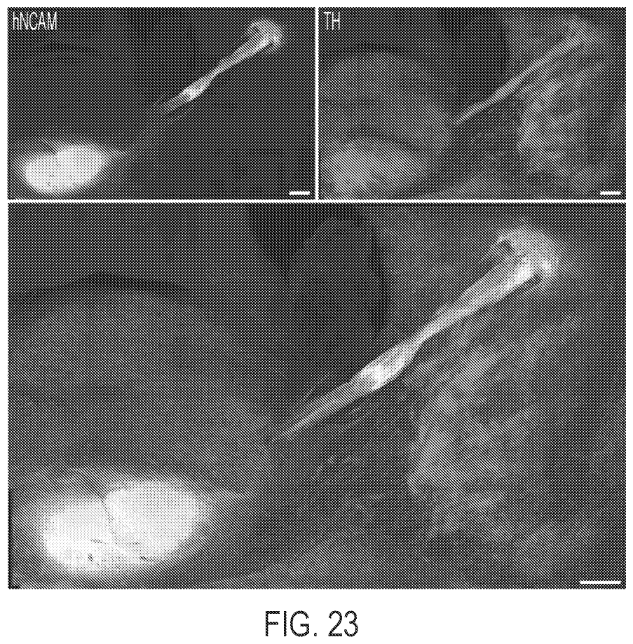

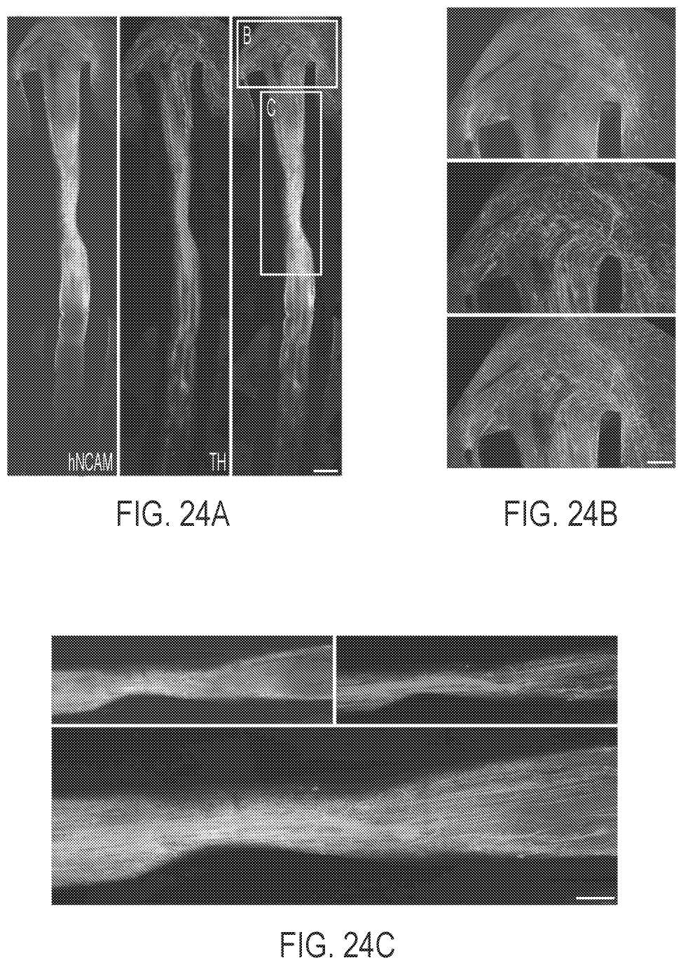

Next, scaled-up human TE-NSPs were generated. FIGS. 22A-22C show expression of dopaminergic neuron markers in long-distance dopaminergic axon tracts in human TE-NSP in vitro having an aggregate with 5.3×10⁴ neurons and 1.5 cm long dopaminergic axon tracts. Human scaled-up TE-NSPs were implanted for 6 months along the nigrostriatal pathway in an athymic rat model of Parkinson's Disease. After 6 months of implantation, human dopaminergic neurons were observed in the TE-NSPs (FIG. 23). Long axon tracts were also preserved within the human TE-NSPs 6 months post-implantation (FIGS. 24C-24C). This data demonstrates in vivo persistence and maintenance of human dopaminergic neurons in the TE-NSPs for at least 6 months.

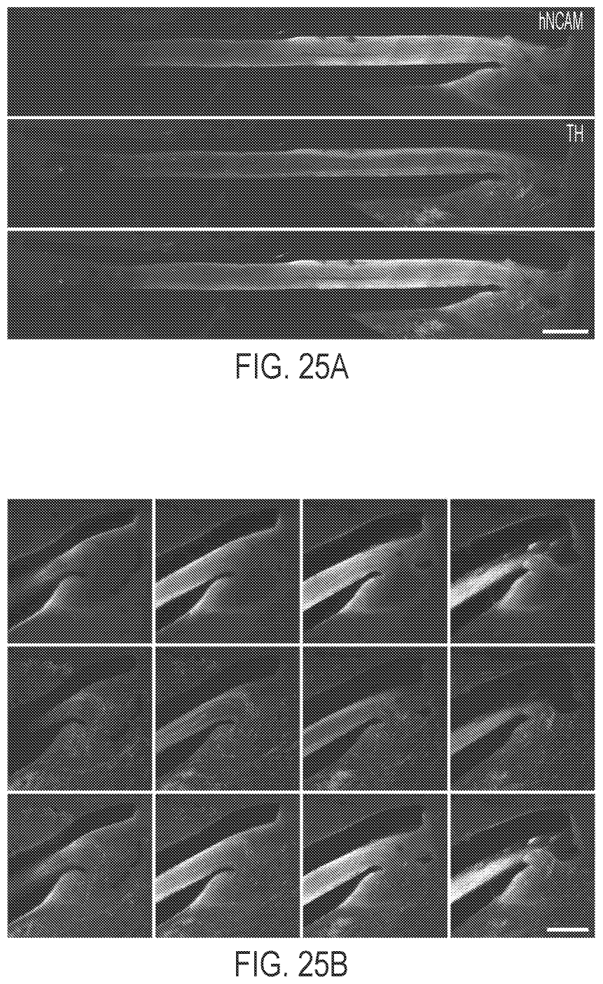

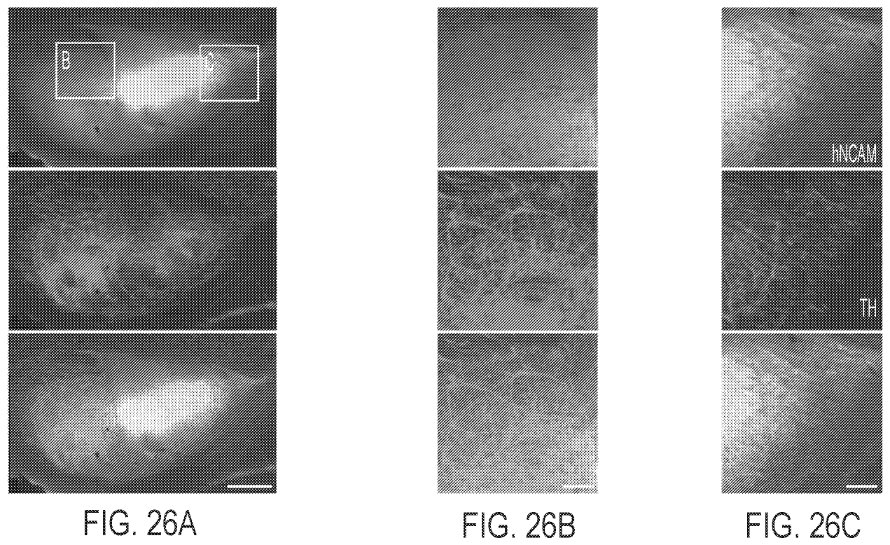

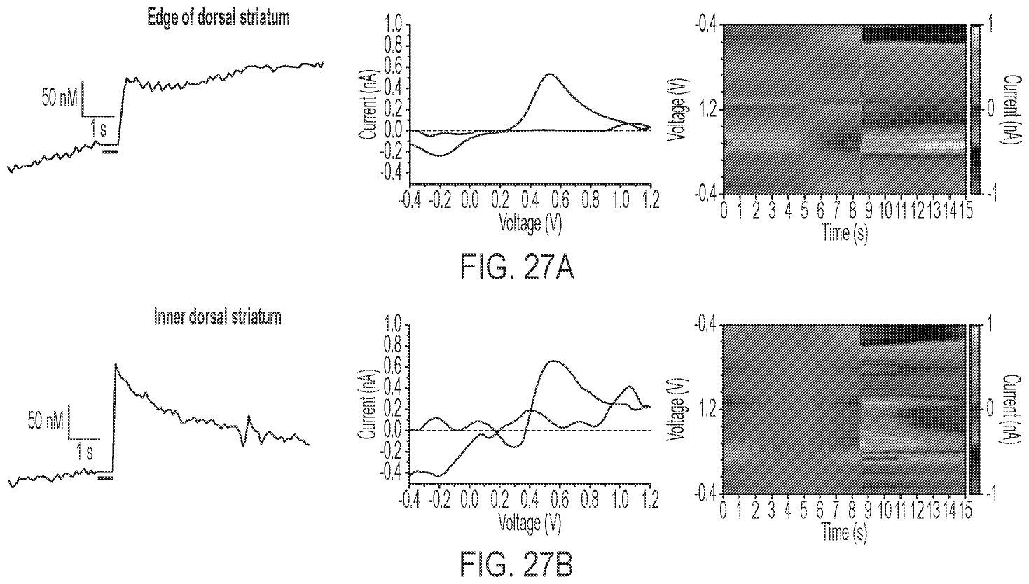

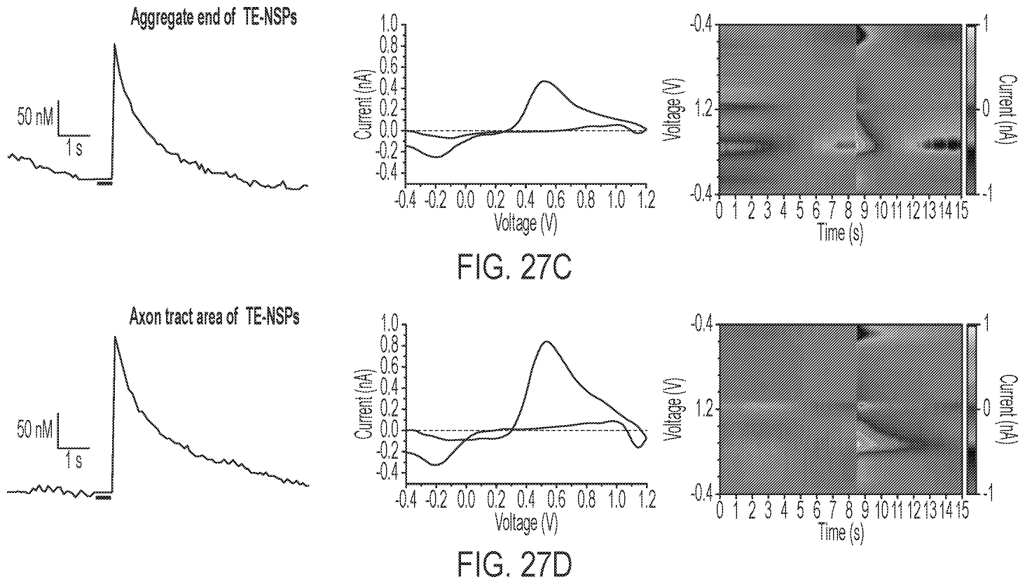

In addition to persisting in vivo for 6 months, the scaled-up TE-NSPs also produced outgrowths of human and dopaminergic projections into the dorsal striatum from the implanted scaled-up TE-NSPs was observed (FIGS. 25A-25B). Survival and growth of the scaled-up TE-NSPs into the region of the substantia nigra is shown in FIGS. 26A-26C. The histological staining shows TH+ neurons and axons (red) and hNCAM+ neurons and axons showing the presence of growth from the nigral end of the implanted TE-NSPs into the host brain. Evoked dopamine release from the implanted TE-NSPs was also evaluated. FIGS. 27A-27D show ex vivo fast-scan cyclic voltammetry (FSCV) measurements of the concentration of dopamine released within implanted TE-NSPs after electrical stimulation at 6 months post-implantation. The electrical stimulation was performed at the indicated sites. The data shows that dopamine was released from the TE-NSPs upon electrical stimulation at all sites tested: edge of dorsal striatum, inner dorsal striatum, aggregate end of TE-NSPs, and axon tract area of TE-NSPs.

In summary, the data in this Example shows the in vivo efficacy of human-scale, human iPSC-derived TE-NSPs. The demonstration of long-term persistence, outgrowth, and functionality of TE-NSPs (e.g., as assessed by reinnervation (see FIG. 20N) and evoked dopamine release (see FIGS. 27A-27D) supports the clinical development of TE-NSP constructs, e.g., for promoting neuronal growth and/or repair.

### LIST OF ENUMERATED EMBODIMENTS

Embodiment 1. A construct comprising a pre-formed neural network, the construct comprising:
   a micro-column comprising an outer sheath comprising a hyaluronic acid (HA) hydrogel, and a core comprising an extracellular matrix (ECM); and
   a plurality of neurons within the micro-column.
Embodiment 2. The construct of Embodiment 1, wherein the construct is biocompatible.
Embodiment 3. The construct of Embodiment 1, wherein the construct is an implantable construct.
Embodiment 4. The construct of Embodiment 1, wherein the hydrogel sheath is cylindrical.
Embodiment 5. The construct of Embodiment 1, wherein the ECM core substantially fills a lumen of the hydrogel sheath.
Embodiment 6. The construct of Embodiment 1, wherein the micro-column is directed along a substantially straight line along its length.
Embodiment 7. The construct of Embodiment 1, wherein the micro-column is directed along a curved path along its length.
Embodiment 8. The construct of any one of Embodiments 1-7, wherein the plurality of neurons have cell bodies substantially localized in proximity to a first end of the micro-column and extend axons longitudinally along at least a portion of a length of the micro-column.
Embodiment 9. The construct of Embodiment 8, wherein the plurality of neurons comprise one or more three dimensional aggregates.
Embodiment 10. The construct of Embodiment 8, wherein the axons are located within and extend longitudinally along a lumen of the hydrogel sheath from the neurons at the first end and towards the opposite end.
Embodiment 11. The construct of Embodiment 8, wherein the axons grow through the ECM of the core and/or along an interface between an inner surface of the hydrogel sheath and the ECM of the core.
Embodiment 12. The construct of Embodiment 8, wherein the neurons and axons extending therefrom have a cyto-architecture that replicates long-range axon tracts present in a subject.
Embodiment 13. The construct of Embodiment 12, wherein the subject is a human subject.
Embodiment 14. The construct of Embodiment 13, wherein the neuronal cells and axons extending therefrom have a cyto-architecture that replicates long-range axon tracts present in a brain of a human subject.
Embodiment 15. The construct of Embodiment 8, wherein the neuronal cells and axons extending therefrom have a cyto-architecture that mimics a native axon pathway between the substantia nigra and the striatum in a brain of a subject.
Embodiment 16. The construct of Embodiment 15, wherein the subject is a human subject.
Embodiment 17. The construct of Embodiment 8, wherein the axon tracts are or have been pre-directed via the micro-column.
Embodiment 18. The construct of Embodiment 8, wherein the neurons together with the axons extending therefrom form a biofabricated micro-tissue.
Embodiment 19. The construct of any one of Embodiments 8-18, wherein the axons of the plurality of neurons extend along at least 50% of the length of the micro-column.
Embodiment 20. The construct of Embodiment 19, wherein the axons extend along at least 75% of the length of the micro-column.
Embodiment 21. The construct of Embodiment 19, wherein the axons extend along 90% of the length of the micro-column.
Embodiment 22. The construct of any one of the preceding Embodiments, wherein an outer diameter of the micro-column ranges from about 500 microns to about 2,500 microns.
Embodiment 23. The construct of any one of the preceding Embodiments, wherein the outer diameter of the micro-column ranges from about 500 to about 1,500 microns.
Embodiment 24. The construct of Embodiment 23, wherein the outer diameter of the micro-column ranges from about 750 to about 1,000 microns.
Embodiment 25. The construct of any one of Embodiments 22-24, wherein the outer diameter is a cross-sectional diameter of the hydrogel sheath.
Embodiment 26. The construct of Embodiment 25, wherein the outer diameter is a cross-sectional diameter of the hydrogel sheath and including any outer coatings thereon.
Embodiment 27. The construct of any one of the preceding Embodiments, wherein an inner diameter of the micro-column ranges from about 250 microns to about 2,000 microns.
Embodiment 28. The construct of Embodiment 27, wherein the inner diameter of the micro-column ranges from about 250 to about 1,000 microns.
Embodiment 29. The construct of Embodiment 27, wherein the inner diameter of the micro-column is about 500 microns.
Embodiment 30. The construct of any one of the preceding Embodiments, wherein the ECM comprises a polysaccharide.
Embodiment 31. The construct of any one of the preceding Embodiments, wherein the ECM comprises one or more members selected from the group consisting of collagen, fibrin, fibronectin, gelatin, hyaluronic acid, laminin, and Matrigel.
Embodiment 32. The construct of any one of the preceding Embodiments, wherein the ECM comprises collagen.
Embodiment 33. The construct of Embodiment 32, wherein the ECM comprises collagen at a concentration ranging from about 0.1 to 10 mg/ml.
Embodiment 34. The construct of Embodiment 33, wherein the ECM comprises collagen at a concentration of about 1 mg/ml.
Embodiment 35. The construct of any one of the preceding Embodiments, wherein the ECM comprises laminin.
Embodiment 36. The construct of Embodiment 35, wherein the ECM comprises laminin at a concentration ranging from about 0.1 to 10 mg/ml.
Embodiment 37. The construct of any one of the preceding Embodiments, wherein the hyaluronic acid (HA) hydrogel is or comprises a cross-linked modified hyaluronic acid.
Embodiment 38. The construct of Embodiment 37, wherein the modified hyaluronic acid is methacrylated HA (MeHA).
Embodiment 39. The construct of Embodiment 38, wherein the hyaluronic acid comprises about 0.5 to about 20% wt MeHA.
Embodiment 40. The construct of Embodiment 39, wherein the hyaluronic acid is or comprises 3-5% MeHA.
Embodiment 41. The construct of Embodiment 37, wherein the modified HA comprises one or more members selected from the group consisting of norbornene-modified HA, acrylated HA, maleimide HA, and hydroxyethyl methacrylate HA.
Embodiment 42. The construct of Embodiment 38, wherein the outer hydrogel sheath is a 3D printed and photopolymerized MeHA cylinder.
Embodiment 43. The construct of any one of the preceding Embodiments, wherein the outer hydrogel sheath comprises one or more hydrolysis sensitive compounds within crosslinks.
Embodiment 44. The construct of Embodiment 43, wherein the one or more hydrolysis sensitive compounds comprise esters.
Embodiment 45. The construct of Embodiment 44, wherein the one or more hydrolysis sensitive compounds comprise lactic acid, caprolactone or an anhydride.
Embodiment 46. The construct of Embodiment 43, wherein the outer hydrogel sheath comprises methacrylated hyaluronic acid doped with the one or more hydrolysis sensitive compounds.
Embodiment 47. The construct of Embodiment 45, wherein one or more hydrolysis sensitive compounds are located between HA and the methacrylate groups.
Embodiment 48. The construct of any one of the preceding Embodiments, wherein the outer hydrogel sheath comprises one or more di-thiol peptides.
Embodiment 49. The construct of Embodiment 48, wherein at least a portion of the one or more di-thiol peptides are sensitive to cleavage.
Embodiment 50. The construct of Embodiment 48, wherein at least a portion of the one or di-thiol peptides are sensitive to cleavage by matrix metalloproteinases expressed by cells.
Embodiment 51. The construct of any one of the preceding Embodiments, wherein the plurality of neurons comprises dopaminergic neurons.
Embodiment 52. The construct of Embodiment 51, wherein at least 50% of the plurality of neurons are dopaminergic neurons.
Embodiment 53. The construct of Embodiment 51, wherein the dopaminergic neurons are obtained via purification.
Embodiment 54. The construct of Embodiment 51, wherein the dopaminergic neurons comprise midbrain dopaminergic neurons.
Embodiment 55. The construct of Embodiment 54, wherein the midbrain dopaminergic neurons comprise A9 neurons.
Embodiment 56. The construct of any one of the preceding Embodiments, wherein the plurality of neurons comprise GABAergic neurons.
Embodiment 57. The construct of Embodiment 56, wherein at least 50% of the plurality of neurons are GABAergic neurons.
Embodiment 58. The construct of Embodiment 56, wherein the GABAergic neurons are obtained via purification.
Embodiment 59. The construct of any one of the preceding Embodiments, wherein the plurality of neurons comprise glutaminergic neurons.
Embodiment 60. The construct of Embodiment 59, wherein at least 50% of the plurality of neurons are glutaminergic neurons.
Embodiment 61. The construct of Embodiment 59, wherein the glutaminergic neurons are obtained via purification.
Embodiment 62. The construct of any one of the preceding Embodiments, wherein the plurality of neurons comprise cholinergic neurons.
Embodiment 63. The construct of Embodiment 62, wherein at least 50% of the plurality of neurons are cholinergic neurons.
Embodiment 64. The construct of Embodiment 62, wherein the cholinergic neurons are obtained via purification.
Embodiment 65. The construct of any one of the preceding Embodiments, wherein the plurality of neurons comprise midbrain neurons.
Embodiment 66. The construct of any one of the preceding Embodiments, wherein the plurality of neurons comprise human neurons.
Embodiment 67. The construct of Embodiment 66, wherein the human neurons comprise induced pluripotent stem cell (iPSC)-derived neurons.
Embodiment 68. The construct of any one of the preceding Embodiments, wherein the plurality of neurons comprise human A9 dopaminergic neurons.
Embodiment 69. The construct of Embodiment 68, wherein the human A9 dopaminergic neurons comprise stem cell-derived human A9 dopaminergic neurons.
Embodiment 70. The construct of Embodiment 69, wherein the stem cells from which the A9 dopaminergic neurons are derived are induced pluripotent stem cells (iPSCs).
Embodiment 71. The construct of any one of the preceding Embodiments, wherein the plurality of neurons comprise at least 50,000 neurons.
Embodiment 72. The construct of Embodiment 71, wherein the plurality of neurons comprise at least 100,000 neurons.
Embodiment 73. The construct of Embodiment 71, wherein the plurality of neurons comprise at least 125,000 neurons.
Embodiment 74. The construct of any one of the preceding Embodiments, wherein the micro-column has a length ranging from about 2 to about 5 centimeters.
Embodiment 75. The construct of Embodiment 74, wherein axons of the plurality of neurons extend a distance ranging from about 2 to about 5 centimeters along the length of the micro-column.
Embodiment 76. The construct of any one of the preceding Embodiments, wherein the micro-column is adapted for implantation along a trajectory encompassing a substantia nigra (SN) region and/or a striatum region of a subject.
Embodiment 77. The construct of Embodiment 76, wherein the SN region is a ventrolateral SN region.
Embodiment 78. The construct of Embodiment 76, wherein the striatum region is a dorsal striatum region.
Embodiment 79. The construct of Embodiment 76, wherein the subject is a human subject.
Embodiment 80. The construct of any one of the preceding Embodiments, wherein the neurons comprise A9 dopaminergic neurons and exhibit dopamine release of at least 50 nM as measured by fast scan cyclic voltammetry.
Embodiment 81. The construct of Embodiment 80, wherein the plurality of neurons releases and/or has a quantity of dopaminergic neurons sufficient to release dopamine at a level sufficient to provide for a level of at least 4 ng/mg in tissue.
Embodiment 82. The construct of Embodiment 81, wherein the dopaminergic neurons release dopamine at a level sufficient to provide for a level of at least 4 ng/mg in tissue within six weeks after implantation in a subject.
Embodiment 83. The construct of any one of the preceding Embodiments, wherein the plurality of neurons provides for increase in 18F-DOPA uptake in a putamen of a subject at a level of about 50-60% of a normal value.
Embodiment 84. The construct of Embodiment 83, wherein the neurons provide for an increase in 18F-DOPA uptake in the putamen of the subject at the level of about 50-60% of a normal value upon implantation in the subject.
Embodiment 85. The construct of Embodiment 83 or 84, wherein an increase at the level of about 50-60% of a normal value is achieved.
Embodiment 86. A method of manufacturing a construct comprising a pre-formed neural network, the method comprising:
   (a) seeding a first end of a micro-column with a plurality of neural precursor cells and/or dopaminergic neurons; and
   (b) culturing the micro-column and plurality of neural cells seeded therein in-vitro.
Embodiment 87. The method of Embodiment 86, wherein the construct is a biocompatible construct.
Embodiment 88. The method of Embodiment 86, wherein the construct is an implantable construct.
Embodiment 89. The method of Embodiment 86, wherein the construct is for use in an in-vitro test bed.
Embodiment 90. The method of Embodiment 86, wherein step (b) comprises causing growth of axons from the neural cells, along a length of the micro-column, toward a second, opposite, end of the micro-column.
Embodiment 91. The method of Embodiment 86, comprising:
   (c) determining axons growth from the plurality of neural cells has reached a particular length; and
   (d) responsive to the particular length of axon growth being determined to have been reached, packaging and/or providing the micro-column for implantation.
Embodiment 92. The method of Embodiment 91, wherein the particular length is a predetermined desired length.
Embodiment 93. The method of Embodiment 91, wherein the particular length ranges from about 2 to about 5 centimeters.
Embodiment 94. The method of Embodiment 91, wherein step (c) comprises imaging the micro-columns and neural cells therein.
Embodiment 95. The method of Embodiment 91, wherein step (c) comprises imaging via microscopy, fast-scan cyclic voltammetry (FSCV), staining, sectioning or measuring axon density.
Embodiment 96. The method of any one of Embodiments 91-94, wherein the plurality of neural cells with which the micro-column is seeded at step (a) comprise neural cell aggregates.
Embodiment 97. The method of Embodiment 96, wherein the neural cell aggregates comprise a plurality of approximately spherical aggregates of neural cells.
Embodiment 98. The method of Embodiment 97, wherein each neural cell aggregate comprises cells at a density ranging from about 100,000 to about 300,000 neurons per aggregate.
Embodiment 99. The method of Embodiment 97, wherein a plurality of the neural cell aggregates exhibit a diameter of at least 500 μm.
Embodiment 100. The method of any one of Embodiments 86 to 89, wherein the micro-column comprises a hydrogel sheath and a core comprising an extracellular matrix (ECM), and wherein the neural cells are seeded to be in direct contact with the ECM of the core.
Embodiment 101. The method of Embodiment 100, wherein the hydrogel sheath comprises MeHA.
Embodiment 102. The method of Embodiment 100, wherein the hydrogel sheath of the micro-column is a 3D printed cylinder.
Embodiment 103. The method of Embodiment 100, wherein the method comprises 3D printing the hydrogel sheath prior to step (a).
Embodiment 104. The method of any one of Embodiments 86-103, comprising, differentiating human induced pluripotent stem cells (iPSCs) for a particular differentiation period prior to step (a), thereby producing differentiated cells and, following differentiating the iPSCs for the particular differentiation period, performing step (a) using the differentiated cells as the neural cells
Embodiment 105. The method of Embodiment 104, comprising seeding the differentiated iPSCs in the micro-column after about 40 dd.
Embodiment 106. The method of Embodiment 104, comprising seeding the differentiated iPSCs in the micro-column after about 11 to about 20 dd.
Embodiment 107. The method of Embodiment 104, comprising seeding the differentiated iPSCs in the micro-column once dopaminergic precursor fate is established and when the cells are usually replanted and matured further.
Embodiment 108. The method of any one of Embodiments 86-107, wherein the micro-column and or cells have one or more features articulated in any one of Embodiments 1 to 85.
Embodiment 109. An in-vitro test bed comprising:
   the construct of any one of Embodiments 1 to 85, comprising a first population of neurons and axons grown therefrom; and
   a second population of neurons, synapsed with the first population.
Embodiment 110. The in-vitro test bed of Embodiment 109, wherein the second population of neurons comprise striatal neurons.
Embodiment 111. The in-vitro test bed of Embodiment 109, wherein the second population of neurons are seeded at an end of the construct opposite to the end at which the first population of neurons were seeded.
Embodiment 112. The in-vitro test bed of Embodiment 109, wherein axons from the first population of neurons extend longitudinally from a first end of the construct along a length of the construct and synapse with the second population seeded at a second, opposite, end of the construct.
Embodiment 113. The in-vitro test bed of Embodiment 109, where cell bodies of the first population are localized in substantial proximity to the first end of the construct.
Embodiment 114. A method of at least partially replacing a population of neurons forming a pathway between the substantia nigra and striatum in a subject, the method comprising implanting at least one construct articulated in any one of Embodiments 1 to 85 into a brain of the subject.
Embodiment 115. The method of Embodiment 114, wherein the method comprises ameliorating one or more conditions of the subject.
Embodiment 116. The method of Embodiment 115, wherein ameliorating the one or more conditions comprises restoring motor function of the subject.
Embodiment 117. The method of Embodiment 115, wherein the ameliorating the one or more conditions comprises reducing pain of the subject.
Embodiment 118. The method of Embodiment 115, wherein the ameliorating the one or more conditions comprises reducing tremors of the subject.
Embodiment 119. The method of any one of Embodiments 114 to 118, comprising implanting the at least a portion of one construct within a substantia nigra of the subject.
Embodiment 120. The method of any one of Embodiments 114 to 119, wherein, following implantation, the neurons of the construct synapse with host neurons in a brain of the subject.
Embodiment 121. The method of Embodiment 120, wherein the host neurons with which the neurons of the construct synapse comprise, medium spiny neurons (MSNs) in a dorsolateral striatum of the subject.
Embodiment 122. The method of any one of Embodiments 114 to 121, wherein the subject is a human subject.
Embodiment 123. The method of any one of Embodiments 114 to 122, wherein implanting the at least one construct comprises using MRI-guided neurosurgery.
Embodiment 124. The method of any one of Embodiments 114 to 123, wherein implanting the at least one construct comprises implanting a plurality of constructs.
Embodiment 125. The method of Embodiment 124, wherein implanting the plurality of constructs comprises implanting a plurality of constructs in a single hemisphere of the brain of the subject.
Embodiment 126. The method of Embodiment 125, wherein implanting the plurality of constructs comprises implanting one or more constructs in each hemisphere of the brain of the subject.
Embodiment 127. The method of Embodiment 126, comprising implanting 1 to 3 constructs in each hemisphere of the brain of the subject.
Embodiment 128. The method of Embodiment 126, wherein implantation in a first hemisphere is performed via a first surgery and implantation in a second hemisphere is performed via a second surgery, performed at a different time than the first surgery.
Embodiment 129. The method of Embodiment 128, wherein the second surgery is performed about 6 months after the first surgery.
Embodiment 130. The construct of any one of Embodiments 1-85, wherein the construct is characterized in that when implanted into a subject, one or more neurons survive in vivo for at least about 9 months, at least about 8 months, at least about 7 months, at least about 6 months, at least about 5 months, at least about 4 months, at least about 3 months, at least about 2 months, or at least about 1 month.
Embodiment 131. The construct of embodiment 130, wherein the one or more neurons maintains an axonal structure or tract, e.g., as assessed in Examples 3 or 4.
Embodiment 132. The construct of Embodiment 130 or 131, wherein the one or more neurons releases dopamine upon electrical stimulation.
Embodiment 133. The construct of any one of Embodiments 1-85, wherein the construct is characterized in that when implanted in a subject, one or more neurons from the construct grow out (e.g., innervate) into a tissue or organ at which the construct is implanted.
Embodiment 134. The construct of embodiment 133, wherein outgrowth of one or more neurons results in an improvement of a physiological characteristic of the tissue or organ.
Embodiment 135. The construct of embodiment 134, wherein improvement of the physiological characteristic of the tissue or organ is as compared to: (1) before the construct was implanted; (2) implantation of an otherwise similar construct without one or more neurons; or (3) implantation of an otherwise similar construct without MeHA.
Embodiment 136. The construct of embodiment 134 or 135, wherein the physiological characteristic comprises: (1) release of a neurostansmitter; (2) presence of a synapse; (3) an ability to transmit a signal; or (4) any combination thereof. EQUIVALENTS Although preferred embodiments of have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A construct comprising a pre-formed neural network, the construct comprising:
a micro-column comprising an outer sheath comprising a hyaluronic acid (HA) hydrogel, and a core comprising an extracellular matrix (ECM); and
a plurality of neurons within the micro-column.

2. The construct of claim 1, wherein the construct is biocompatible.

3. The construct of claim 1, wherein the construct is an implantable construct.

4. The construct of claim 1, wherein the hydrogel sheath is cylindrical.

5. The construct of claim 1, wherein the ECM core substantially fills a lumen of the hydrogel sheath.

6. The construct of claim 1, wherein the micro-column is directed along a substantially straight line along its length.

7. The construct of claim 1, wherein the micro-column is directed along a curved path along its length.

8. The construct of claim 1, wherein the plurality of neurons have cell bodies substantially localized in proximity to a first end of the micro-column and extend axons longitudinally along at least a portion of a length of the micro-column.

9. The construct of claim 8, wherein the plurality of neurons comprise one or more three dimensional aggregates.

10. The construct of claim 8, wherein the axons are located within and extend longitudinally along a lumen of the hydrogel sheath from the neurons at the first end and towards the opposite end.

11. The construct of claim 8, wherein the axons grow through the ECM of the core and/or along an interface between an inner surface of the hydrogel sheath and the ECM of the core.

12. The construct of claim 8, wherein the neurons and axons extending therefrom have a cyto-architecture that replicates long-range axon tracts present in a subject.

13. The construct of claim 12, wherein the subject is a human subject.

14. The construct of claim 13, wherein the neuronal cells and axons extending therefrom have a cyto-architecture that replicates long-range axon tracts present in a brain of a human subject.

15. The construct of claim 8, wherein the neuronal cells and axons extending therefrom have a cyto-architecture that mimics a native axon pathway between the substantia nigra and the striatum in a brain of a subject.

16. The construct of claim 15, wherein the subject is a human subject.

17. The construct of claim 8, wherein the axon tracts are or have been pre-directed via the micro-column.

18. The construct of claim 8, wherein the neurons together with the axons extending therefrom form a biofabricated micro-tissue.

19. The construct of claim 8, wherein the axons of the plurality of neurons extend along at least 50% of the length of the micro-column.

20. The construct of claim 19, wherein the axons extend along at least 75% of the length of the micro-column.

21. The construct of claim 19, wherein the axons extend along 90% of the length of the micro-column.

22. The construct of claim 1, wherein an outer diameter of the micro-column ranges from about 500 microns to about 2,500 microns.

23. The construct of claim 1, wherein the outer diameter of the micro-column ranges from about 500 to about 1,500 microns.

24. The construct of claim 23, wherein the outer diameter of the micro-column ranges from about 750 to about 1,000 microns.

25. The construct of claim 22, wherein the outer diameter is a cross-sectional diameter of the hydrogel sheath.

26. The construct of claim 25, wherein the outer diameter is a cross-sectional diameter of the hydrogel sheath and including any outer coatings thereon.

27. The construct of claim 1, wherein an inner diameter of the micro-column ranges from about 250 microns to about 2,000 microns.

28. The construct of claim 27, wherein the inner diameter of the micro-column ranges from about 250 to about 1,000 microns.

29. The construct of claim 27, wherein the inner diameter of the micro-column is about 500 microns.

30. The construct of claim 1, wherein the ECM comprises a polysaccharide.

31. The construct of claim 1, wherein the ECM comprises one or more members selected from the group consisting of collagen, fibrin, fibronectin, gelatin, hyaluronic acid, laminin, and Matrigel.

32. The construct of claim 1, wherein the ECM comprises collagen.

33. The construct of claim 32, wherein the ECM comprises collagen at a concentration ranging from about 0.1 to 10 mg/ml.

34. The construct of claim 33, wherein the ECM comprises collagen at a concentration of about 1 mg/ml.

35. The construct of claim 1, wherein the ECM comprises laminin.

36. The construct of claim 35, wherein the ECM comprises laminin at a concentration ranging from about 0.1 to 10 mg/ml.

37. The construct of claim 1, wherein the hyaluronic acid (HA) hydrogel is or comprises a cross-linked modified hyaluronic acid.

38. The construct of claim 37, wherein the modified hyaluronic acid is methacrylated HA (MeHA).

39. The construct of claim 38, wherein the hyaluronic acid comprises about 0.5 to about 20% wt MeHA.

40. The construct of claim 39, wherein the hyaluronic acid is or comprises 3-5% MeHA.

41. The construct of claim 37, wherein the modified HA comprises one or more members selected from the group consisting of norbornene-modified HA, acrylated HA, maleimide HA, and hydroxyethyl methacrylate HA.

42. The construct of claim 38, wherein the outer hydrogel sheath is a 3D printed and photopolymerized MeHA cylinder.

43. The construct of claim 1, wherein the outer hydrogel sheath comprises one or more hydrolysis sensitive compounds within crosslinks.

44. The construct of claim 43, wherein the one or more hydrolysis sensitive compounds comprise esters.

45. The construct of claim 44, wherein the one or more hydrolysis sensitive compounds comprise lactic acid, caprolactone or an anhydride.

46. The construct of claim 43, wherein the outer hydrogel sheath comprises methacrylated hyaluronic acid doped with the one or more hydrolysis sensitive compounds.

47. The construct of claim 45, wherein one or more hydrolysis sensitive compounds are located between HA and the methacrylate groups.

48. The construct of claim 1, wherein the outer hydrogel sheath comprises one or more di-thiol peptides.

49. The construct of claim 48, wherein at least a portion of the one or more di-thiol peptides are sensitive to cleavage.

50. The construct of claim 48, wherein at least a portion of the one or di-thiol peptides are sensitive to cleavage by matrix metalloproteinases expressed by cells.

51. The construct of claim 1, wherein the plurality of neurons comprises dopaminergic neurons.

52. The construct of claim 51, wherein at least 50% of the plurality of neurons are dopaminergic neurons.

53. The construct of claim 51, wherein the dopaminergic neurons are obtained via purification.

54. The construct of claim 51, wherein the dopaminergic neurons comprise midbrain dopaminergic neurons.

55. The construct of claim 54, wherein the midbrain dopaminergic neurons comprise A9 neurons.

56. The construct of claim 1, wherein the plurality of neurons comprise GABAergic neurons.

57. The construct of claim 56, wherein at least 50% of the plurality of neurons are GABAergic neurons.

58. The construct of claim 56, wherein the GABAergic neurons are obtained via purification.

59. The construct of claim 1, wherein the plurality of neurons comprise glutaminergic neurons.

60. The construct of claim 59, wherein at least 50% of the plurality of neurons are glutaminergic neurons.

61. The construct of claim 59, wherein the glutaminergic neurons are obtained via purification.

62. The construct of claim 1, wherein the plurality of neurons comprise cholinergic neurons.

63. The construct of claim 62, wherein at least 50% of the plurality of neurons are cholinergic neurons.

64. The construct of claim 62, wherein the cholinergic neurons are obtained via purification.

65. The construct of claim 1, wherein the plurality of neurons comprise midbrain neurons.

66. The construct of claim 1, wherein the plurality of neurons comprise human neurons.

67. The construct of claim 66, wherein the human neurons comprise induced pluripotent stem cell (iPSC)-derived neurons.

68. The construct of claim 1, wherein the plurality of neurons comprise human A9 dopaminergic neurons.

69. The construct of claim 68, wherein the human A9 dopaminergic neurons comprise stem cell-derived human A9 dopaminergic neurons.

70. The construct of claim 69, wherein the stem cells from which the A9 dopaminergic neurons are derived are induced pluripotent stem cells (iPSCs).

71. The construct of claim 1, wherein the plurality of neurons comprise at least 50,000 neurons.

72. The construct of claim 71, wherein the plurality of neurons comprise at least 100,000 neurons.

73. The construct of claim 71, wherein the plurality of neurons comprise at least 125,000 neurons.

74. The construct of claim 1, wherein the micro-column has a length ranging from about 2 to about 5 centimeters.

75. The construct of claim 74, wherein axons of the plurality of neurons extend a distance ranging from about 2 to about 5 centimeters along the length of the micro-column.

76. The construct of claim 1, wherein the micro-column is adapted for implantation along a trajectory encompassing a substantia nigra (SN) region and a striatum region of a subject.

77. The construct of claim 76, wherein the SN region is a ventrolateral SN region.

78. The construct of claim 76, wherein the striatum region is a dorsal striatum region.

79. The construct of claim 76, wherein the subject is a human subject.

80. The construct of claim 1, wherein the neurons comprise A9 dopaminergic neurons and exhibit dopamine release of at least 50 nM as measured by fast scan cyclic voltammetry.

81. The construct of claim 80, wherein the plurality of neurons releases and/or has a quantity of dopaminergic neurons sufficient to release dopamine at a level sufficient to provide for a level of at least 4 ng/mg in tissue.

82. The construct of claim 81, wherein the dopaminergic neurons release dopamine at a level sufficient to provide for a level of at least 4 ng/mg in tissue within six weeks after implantation in a subject.

83. The construct of claim 1, wherein the plurality of neurons provides for increase in 18F-DOPA uptake in a putamen of a subject at a level of about 50-60% of a normal value.

84. The construct of claim 83, wherein the neurons provide for an increase in 18F-DOPA uptake in the putamen of the subject at the level of about 50-60% of a normal value upon implantation in the subject.

85. The construct of claim 83, wherein an increase at the level of about 50-60% of a normal value is achieved.

86. A method of manufacturing a construct comprising a pre-formed neural network, the method comprising:
(a) seeding a first end of a micro-column with a plurality of neural precursor cells and/or dopaminergic neurons; and
(b) culturing the micro-column and plurality of neural cells seeded therein in-vitro.

87. The method of claim 86, wherein the construct is a biocompatible construct.

88. The method of claim 86, wherein the construct is an implantable construct.

89. The method of claim 86, wherein the construct is for use in an in-vitro test bed.

90. The method of claim 86, wherein step (b) comprises causing growth of axons from the neural cells, along a length of the micro-column, toward a second, opposite, end of the micro-column.

91. The method of claim 86, comprising:
(c) determining axons growth from the plurality of neural cells has reached a particular length; and
(d) responsive to the particular length of axon growth being determined to have been reached, packaging and/or providing the micro-column for implantation.

92. The method of claim 91, wherein the particular length is a predetermined desired length.

93. The method of claim 91, wherein the particular length ranges from about 2 to about 5 centimeters.

94. The method of claim 91, wherein step (c) comprises imaging the micro-columns and neural cells therein.

95. The method of claim 91, wherein step (c) comprises imaging via microscopy, fast-scan cyclic voltammetry (FSCV), staining, sectioning or measuring axon density.

96. The method of claim 91, wherein the plurality of neural cells with which the micro-column is seeded at step (a) comprise neural cell aggregates.

97. The method of claim 96, wherein the neural cell aggregates comprise a plurality of approximately spherical aggregates of neural cells.

98. The method of claim 97, wherein each neural cell aggregate comprises cells at a density ranging from about 100,000 to about 300,000 neurons per aggregate.

99. The method of claim 97, wherein a plurality of the neural cell aggregates exhibit a diameter of at least 500 μm.

100. The method of claim 86, wherein the micro-column comprises a hydrogel sheath and a core comprising an extracellular matrix (ECM), and wherein the neural cells are seeded to be in direct contact with the ECM of the core.

101. The method of claim 100, wherein the hydrogel sheath comprises MeHA.

102. The method of claim 100, wherein the hydrogel sheath of the micro-column is a 3D printed cylinder.

103. The method of claim 100, wherein the method comprises 3D printing the hydrogel sheath prior to step (a).

104. The method of claim 86, comprising, differentiating human induced pluripotent stem cells (iPSCs) for a particular differentiation period prior to step (a), thereby producing differentiated cells and, following differentiating the iPSCs for the particular differentiation period, performing step (a) using the differentiated cells as the neural cells

105. The method of claim 104, comprising seeding the differentiated iPSCs in the micro-column after about 40 dd.

106. The method of claim 104, comprising seeding the differentiated iPSCs in the micro-column after about 11 to about 20 dd.

107. The method of claim 104, comprising seeding the differentiated iPSCs in the micro-column once dopaminergic precursor fate is established and when the cells are usually replanted and matured further.

108. (canceled)

109. An in-vitro test bed comprising:
the construct of claim 1, comprising a first population of neurons and axons grown therefrom; and
a second population of neurons, synapsed with the first population.

110. The in-vitro test bed of claim 109, wherein the second population of neurons comprise striatal neurons.

111. The in-vitro test bed of claim 109, wherein the second population of neurons are seeded at an end of the construct opposite to the end at which the first population of neurons were seeded.

112. The in-vitro test bed of claim 109, wherein axons from the first population of neurons extend longitudinally from a first end of the construct along a length of the construct and synapse with the second population seeded at a second, opposite, end of the construct.

113. The in-vitro test bed of claim 109, where cell bodies of the first population are localized in substantial proximity to the first end of the construct.

114. A method of at least partially replacing a population of neurons forming a pathway between the substantia nigra and striatum in a subject, the method comprising implanting at least one construct articulated in claim 1 into a brain of the subject.

115. The method of claim 114, wherein the method comprises ameliorating one or more conditions of the subject.

116. The method of claim 115, wherein ameliorating the one or more conditions comprises restoring motor function of the subject.

117. The method of claim 115, wherein the ameliorating the one or more conditions comprises reducing pain of the subject.

118. The method of claim 115, wherein the ameliorating the one or more conditions comprises reducing tremors of the subject.

119. The method of claim 114, comprising implanting the at least a portion of one construct within a substantia nigra of the subject.

120. The method of claim 114, wherein, following implantation, the neurons of the construct synapse with host neurons in a brain of the subject.

121. The method of claim 120, wherein the host neurons with which the neurons of the construct synapse comprise, medium spiny neurons (MSNs) in a dorsolateral striatum of the subject.

122. The method of claim 114, wherein the subject is a human subject.

123. The method of claim 114, wherein implanting the at least one construct comprises using MRI-guided neurosurgery.

124. The method of claim 114, wherein implanting the at least one construct comprises implanting a plurality of constructs.

125. The method of claim 124, wherein implanting the plurality of constructs comprises implanting a plurality of constructs in a single hemisphere of the brain of the subject.

126. The method of claim 125, wherein implanting the plurality of constructs comprises implanting one or more constructs in each hemisphere of the brain of the subject.

127. The method of claim 126, comprising implanting 1 to 3 constructs in each hemisphere of the brain of the subject.

128. The method of claim 126, wherein implantation in a first hemisphere is performed via a first surgery and implantation in a second hemisphere is performed via a second surgery, performed at a different time than the first surgery.

129. The method of claim 128, wherein the second surgery is performed about 6 months after the first surgery.
